SEQUENCE LISTING (140 sequences)

SEQ ID NO: 1 - DNA, 19 bp, Artificial Sequence
  Description of Artificial Sequence Synthetic oligonucleotide primer
gagcccgcggcgcattagc

SEQ ID NO: 2 - DNA, 17 bp, Artificial Sequence
  Description of Artificial Sequence Synthetic oligonucleotide probe
gcgacgatgcgtagccg

SEQ ID NO: 3 - DNA, 16 bp, Artificial Sequence
  Description of Artificial Sequence Synthetic oligonucleotide
cgcaatgggcgcaagc

SEQ ID NO: 4 - DNA, 16 bp, Artificial Sequence
  Description of Artificial Sequence Synthetic oligonucleotide primer
gcttgcgcccattgcg

SEQ ID NO: 5 - DNA, 19 bp, Artificial Sequence
  Description of Artificial Sequence Synthetic oligonucleotide primer
gagcaacgccgcgtgagcg

SEQ ID NO: 6 - DNA, 16 bp, Artificial Sequence
  Description of Artificial Sequence Synthetic oligonucleotide probe
cttcgggttgtaaagc

SEQ ID NO: 7 - DNA, 15 bp, Artificial Sequence
  Description of Artificial Sequence Synthetic oligonucleotide
cggctaactacgtgc

SEQ ID NO: 8 - DNA, 15 bp, Artificial Sequence
  Description of Artificial Sequence Synthetic oligonucleotide primer
gcacgtagttagccg

SEQ ID NO: 9 - DNA, 18 bp, Artificial Sequence
  Description of Artificial Sequence Synthetic oligonucleotide primer
agtgctggagaggcaagg

SEQ ID NO: 10 - DNA, 17 bp, Artificial Sequence
  Description of Artificial Sequence Synthetic oligonucleotide probe
ctggacagtgactgacg

SEQ ID NO: 11 - DNA, 20 bp, Artificial Sequence
  Description of Artificial Sequence Synthetic oligonucleotide
gcacgaaagcgtggggagca

SEQ ID NO: 12 - DNA, 20 bp, Artificial Sequence
  Description of Artificial Sequence Synthetic oligonucleotide primer
tgctccccacgctttcgtgc

SEQ ID NO: 13 - DNA, 20 bp, Artificial Sequence
  Description of Artificial Sequence Synthetic oligonucleotide primer
ggagtacggtcgcaagactg

SEQ ID NO: 14 - DNA, 17 bp, Artificial Sequence
  Description of Artificial Sequence Synthetic oligonucleotide probe
cgcacaagcagtggagc

SEQ ID NO: 15 - DNA, 14 bp, Artificial Sequence
  Description of Artificial Sequence Synthetic oligonucleotide
cagggcttgacatc

SEQ ID NO: 16 - DNA, 14 bp, Artificial Sequence
  Description of Artificial Sequence Synthetic oligonucleotide primer
gatgtcaagccctg

SEQ ID NO: 17 - DNA, 19 bp, Artificial Sequence
  Description of Artificial Sequence Synthetic oligonucleotide primer
ggcgtaagtcggaggaagg

SEQ ID NO: 18 - DNA, 19 bp, Artificial Sequence
  Description of Artificial Sequence Synthetic oligonucleotide probe
atgtcctgggctacacacg

SEQ ID NO: 19 - DNA, 19 bp, Artificial Sequence
  Description of Artificial Sequence Synthetic oligonucleotide primer
gcctgcaatccgaactacc

SEQ ID NO: 20 - DNA, 19 bp, Artificial Sequence
  Description of Artificial Sequence Synthetic oligonucleotide primer
cgtagttcggattgcaggc

SEQ ID NO: 21 - DNA, 20 bp, Artificial Sequence
  Description of Artificial Sequence Synthetic oligonucleotide probe
cggaattgctagtaatcgcg

SEQ ID NO: 22 - DNA, 18 bp, Artificial Sequence
  Description of Artificial Sequence Synthetic oligonucleotide
cacgagagtcggcaacac

SEQ ID NO: 23 - DNA, 18 bp, Artificial Sequence
  Description of Artificial Sequence Synthetic oligonucleotide primer
gtgttgccgactctcgtg

SEQ ID NO: 24 - DNA, 16 bp, Artificial Sequence
  Description of Artificial Sequence Synthetic oligonucleotide primer
gatgattggggtgaag

SEQ ID NO: 25 - DNA, 16 bp, Artificial Sequence
  Description of Artificial Sequence Synthetic oligonucleotide primer
atgcagagytcgaacg

SEQ ID NO: 26 - DNA, 19 bp, Artificial Sequence
  Description of Artificial Sequence Synthetic oligonucleotide probe
tcgagaaggacgtcaccgc

SEQ ID NO: 27 - DNA, 17 bp, Artificial Sequence
  Description of Artificial Sequence Synthetic oligonucleotide primer
aagctgccgaarcactc

SEQ ID NO: 28 - DNA, 17 bp, Artificial Sequence
  Description of Artificial Sequence Synthetic oligonucleotide primer
tactggtgggggccgct

SEQ ID NO: 29 - DNA, 17 bp, Artificial Sequence
  Description of Artificial Sequence Synthetic oligonucleotide primer
tactggtgggcgccgct

SEQ ID NO: 30 - DNA, 19 bp, Artificial Sequence
  Description of Artificial Sequence Synthetic oligonucleotide probe
atggaagcggagtacgtcc

SEQ ID NO: 31 - DNA, 20 bp, Artificial Sequence
  Description of Artificial Sequence Synthetic oligonucleotide probe
atggaagcggagtacgtcct

SEQ ID NO: 32 - DNA, 19 bp, Artificial Sequence
  Description of Artificial Sequence Synthetic oligonucleotide probe
atggaagcggaatatgtgc

SEQ ID NO: 33 - DNA, 20 bp, Artificial Sequence
  Description of Artificial Sequence Synthetic oligonucleotide probe
atggaagcggaatatgtgct

SEQ ID NO: 34 - DNA, 14 bp, Artificial Sequence
  Description of Artificial Sequence Synthetic oligonucleotide primer
cgcgaggacggcac

SEQ ID NO: 35 - DNA, 18 bp, Artificial Sequence
  Description of Artificial Sequence Synthetic oligonucleotide primer
cgcgaggacggcacgtgg

SEQ ID NO: 36 - DNA, 14 bp, Artificial Sequence
  Description of Artificial Sequence Synthetic oligonucleotide primer
cgcgaagacggcac

SEQ ID NO: 37 - DNA, 18 bp, Artificial Sequence
  Description of Artificial Sequence Synthetic oligonucleotide primer
cgcgaagacggcacctgg

SEQ ID NO: 38 - DNA, 17 bp, Artificial Sequence
  Description of Artificial Sequence Synthetic oligonucleotide primer
caaaaggcgctcgactg

SEQ ID NO: 39 - DNA, 18 bp, Artificial Sequence
  Description of Artificial Sequence Synthetic oligonucleotide primer
caaaaggcgctcgactgg

SEQ ID NO: 40 - DNA, 22 bp, Artificial Sequence
  Description of Artificial Sequence Synthetic oligonucleotide primer
caaaaggcgctcgactgggtcg

SEQ ID NO: 41 - DNA, 17 bp, Artificial Sequence
  Description of Artificial Sequence Synthetic oligonucleotide primer
caaaagtcgctcgactg

SEQ ID NO: 42 - DNA, 18 bp, Artificial Sequence
  Description of Artificial Sequence Synthetic oligonucleotide primer
caaaagtcgctcgactgg

SEQ ID NO: 43 - DNA, 22 bp, Artificial Sequence
  Description of Artificial Sequence Synthetic oligonucleotide primer
caaaagtcgctcgactggctcg

SEQ ID NO: 44 - DNA, 18 bp, Artificial Sequence
  Description of Artificial Sequence Synthetic oligonucleotide probe
ggacggcggctggggcga

SEQ ID NO: 45 - DNA, 21 bp, Artificial Sequence
  Description of Artificial Sequence Synthetic oligonucleotide probe
ggacggcggctggggcgagga

SEQ ID NO: 46 - DNA, 27 bp, Artificial Sequence
  Description of Artificial Sequence Synthetic oligonucleotide probe
ggacggcggctggggcgaggactgccg

SEQ ID NO: 47 - DNA, 18 bp, Artificial Sequence
  Description of Artificial Sequence Synthetic oligonucleotide probe
ggatggcggttggggtga

SEQ ID NO: 48 - DNA, 21 bp, Artificial Sequence
  Description of Artificial Sequence Synthetic oligonucleotide probe
ggatggcggttggggtgaaga

SEQ ID NO: 49 - DNA, 27 bp, Artificial Sequence
  Description of Artificial Sequence Synthetic oligonucleotide probe
ggatggcggttggggtgaagattgccg

SEQ ID NO: 50 - DNA, 16 bp, Artificial Sequence
  Description of Artificial Sequence Synthetic oligonucleotide probe
tgatggcgctcatcgc

SEQ ID NO: 51 - DNA, 23 bp, Artificial Sequence
  Description of Artificial Sequence Synthetic oligonucleotide primer
tgatggcgctcatcgcgggcggc

SEQ ID NO: 52 - DNA, 25 bp, Artificial Sequence
  Description of Artificial Sequence Synthetic oligonucleotide primer
accccgtcgcagacggcctgggcgc

SEQ ID NO: 53 - DNA, 25 bp, Artificial Sequence
  Description of Artificial Sequence Synthetic oligonucleotide primer
acaccgtcgcagaccgcctgggcgt

SEQ ID NO: 54 - DNA, 19 bp, Artificial Sequence
  Description of Artificial Sequence Synthetic oligonucleotide primer
gtggtgctagcatttgctg

SEQ ID NO: 55 - DNA, 18 bp, Artificial Sequence
  Description of Artificial Sequence Synthetic oligonucleotide primer
gttagactcgctggctcc

SEQ ID NO: 56 - DNA, 23 bp, Artificial Sequence
  Description of Artificial Sequence Synthetic oligonucleotide probe
tttcaagccgatggaagtttgas

SEQ ID NO: 57 - DNA, 21 bp, Artificial Sequence
  Description of Artificial Sequence Synthetic oligonucleotide probe
cggtttcaagccgatggaagt

SEQ ID NO: 58 - DNA, 30 bp, Artificial Sequence
  Description of Artificial Sequence Synthetic oligonucleotide primer
cctactaaatagggtgctagcatttgctgg

SEQ ID NO: 59 - DNA, 26 bp, Artificial Sequence
  Description of Artificial Sequence Synthetic oligonucleotide primer
ctaaatagggtgctagcatttgctgg

SEQ ID NO: 60 - DNA, 25 bp, Artificial Sequence
  Description of Artificial Sequence Synthetic oligonucleotide probe
cggtttcaagccgatggaagtttga

SEQ ID NO: 61 - DNA, 17 bp, Artificial Sequence
  Description of Artificial Sequence Synthetic oligonucleotide primer
ccgctggcttcttaggg

SEQ ID NO: 62 - DNA, 19 bp, Artificial Sequence
  Description of Artificial Sequence Synthetic oligonucleotide primer
agggccagcgagtacatca

SEQ ID NO: 63 - DNA, 20 bp, Artificial Sequence
  Description of Artificial Sequence Synthetic oligonucleotide probe
ctcaagccgatggaagtgcg

SEQ ID NO: 64 - DNA, 20 bp, Artificial Sequence
  Description of Artificial Sequence Synthetic oligonucleotide primer
ggnggntggatgttycargc

SEQ ID NO: 65 - DNA, 18 bp, Artificial Sequence
  Description of Artificial Sequence Synthetic oligonucleotide primer
ytcnccccanccnccrtc

SEQ ID NO: 66 - DNA, 19 bp, Artificial Sequence
  Description of Artificial Sequence Synthetic oligonucleotide primer
cgtagttcggattgcaggc

SEQ ID NO: 67 - DNA, 19 bp, Artificial Sequence
  Description of Artificial Sequence Synthetic oligonucleotide probe
cggaattgctagtaatcgc

SEQ ID NO: 68 - DNA, 18 bp, Artificial Sequence
  Description of Artificial Sequence Synthetic oligonucleotide primer
cacgagagtcggcaacac

SEQ ID NO: 69 - DNA, 21 bp, Artificial Sequence
  Description of Artificial Sequence Synthetic oligonucleotide primer
tgcatggccgttcttagttgg

SEQ ID NO: 70 - DNA, 19 bp, Artificial Sequence
  Description of Artificial Sequence Synthetic oligonucleotide primer
gtgtgtacaaagggcaggg

SEQ ID NO: 71 - DNA, 19 bp, Artificial Sequence
  Description of Artificial Sequence Synthetic oligonucleotide primer
cgtagttcggattgcaggc

SEQ ID NO: 72 - DNA, 18 bp, Artificial Sequence
  Description of Artificial Sequence Synthetic oligonucleotide primer
gtgttgccgactctcgtg

SEQ ID NO: 73 - DNA, 19 bp, Artificial Sequence
  Description of Artificial Sequence Synthetic oligonucleotide probe
cggaattgctagtaatcgc

SEQ ID NO: 74 - DNA, 1500 bp, Alicyclobacillus acidocaldarius
agagtttgatcctggctcaggacgaacgctggcggcgtgcctaatacatgcaagtcgagcgggtctcttcggaggccagcggcggacgggtgaggaacacgtgggtaatctgcctttcaggccggaataacgcccggaaacgggcgctaaagccggatacgcccgcgaggaggcatcttcttgcgggggaaggcccaattgggtcgctgagagaggagcccgcggcgcattagctagttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatgggggatggaaagccccgtgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctttggaaactgcttgacttgagtgctggagaggcaaggggaattccacgtgtagcggtgaaatgcgtagagatgtggaggaataccagtggcgaargcgccttgctggacagtgactgacgctgaggcacgaaagcgtggggagcaaacaggattagataccctggtagtccacgccgtaaacgatgagtgctaggtgttggggggacacaccccagtgccgaaggaaamccaataagcactccgcctggggagtacggtcgcaagactgaaactcaaaggaattgacgggggcccgcacaagcagtggagcatgtggtttaaatcgaagcaacgcgaagaaccttaccagggcttgacatccctctgacaccctcagagatgaggggtcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgccggcgtaagtcggaggaaggcggggatgacgtcaaatcatcatgcccctgatgtcctgggctacacacgtgctacaatgggcggaacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaacccctgtggggagccagccgccgaaggtggggtcgatgattggggtgaagtcgtaacaaggtagccgt

SEQ ID NO: 75 - DNA, 1519 bp, Alicyclobacillus acidoterrestris
  modified_base (549)..(549): a, c, g, or t
gacgaacgctggcggcgtgcctaatacatgcaagtcgagcgagcccttcggggctagcggcggacgggtgagtaacacgtgggcaatccgcctttcagactggaataacactcggaaacgggtgctaatgccggataatacacgggtaggcatctacttgtgttgaaagatgcaactgcatcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactggggcgtaaagcgtgcgtangcggttgtgtaagtctgaactgaaagtccaaggctcnaccttgggnatgctttggaaactgcatggacttgagtgctggagaggcnaggcnaattccncgtgttaccggtgnaaatgcgntanatatgtggaggaataccagtggcnaangcgcctttgctggacagtggactgacgctgaaggcacgaaaancgtggggancaacnggattanatccccnaangcgnggggaagcaaacaggattagattcccnttgtagtcccgccccgtaancnatgagtacttagttgttgggggaacacaccccantgcggnggaaacccaataagcactccgcctggggagtgcggtcncaagactgaanctcaaaggaattgacgggggcccgcacaagcagtggagcatntggtttaattcgaagcaacgcgaagaaccttaccagggctngacatccctctgaccggtgcagagatgtaccttcccttcggggcagaggagacaggtggtgcatggttgtcgtcagctcgtgtcgtgagatgttgggttaagtcccgcaacgagcgcaacccttgatctgtgttaccagcacgttgtggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcaaaacctaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaaccgttatggagccagccgccgaaggtggggttgatgattggggtgaagtcgtaacaaggtagccgt

SEQ ID NO: 76 - DNA, 1497 bp, Alicyclobacillus cycloheptanicus
  modified_base (967): a, t, c or g
agagtttgatcctggctcaggacgaacgctggcggcgtgcctaatacatgcaagtcgagcggacccttcggggtcagcggcggacgggtgagtaacacgtgggtaatctgcccaactgaccggaataacgcctggaaacgggtgctaatgccggataggcagcgagcaggcatctgctcgctgggaaaggtgcaagtgcaccgcagatggaggagcccgcggcgcattagctggttggtggggtaacggctcaccaaggcgacgatgcgtagccgacctgagagggtggacggccacactgggactgagacacggcccagactcctacgggaggcagcagtagggaatcttccgcaatgggcgcaagcctgacggagcaacgccgcgtgagcgaagaaggccttcgggttgtaaagctcagtcactcgggaagagcggcaaggggagtggaaagccccttgagagacggtaccgagagaggaagccccggctaactacgtgccagcagccgcggtaatacgtagggggcaagcgttgtccggaatcactgggcgtaaagcgtgcgtaggcggttgcgtgtgtccggggtgaaagtccagggctcaaccctgggaatgccttggaaactgcgtaacttgagtgctggagaggcaaggggaattccgcgtgtagcggtggaatgcgtagatatgcggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttancagggctcgacatccccctgacagccgcagagatgcggtttcccttcggggcaggggagacaggtggtgcatggttgtcgtcagctcgtgtcgtgagatgttgggttaagtcccgcaacgagcgcaacccttgaactgtgttaccagcacgtgaaggtggggactcacagttgactgccggcgtaagtcggaggaaggcggggatgacgtcaaatcatcatgccctttatgtcctgggctacacacgtgctacaatgggcggtacaacgggaagcgagaccgcgaggtggagcaaacccctgaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtggggtaacccgtcagggagccagccgccgaaggtggggttgatgattggggtgaagtcgtaacaaggtagccgt

SEQ ID NO: 77 - DNA, 1546 bp, Artificial Sequence
  Description of Artificial Sequence Figure 1 consensus sequence
nnnnnnnnnnnnnnnnnnnngacgaacgctggcggcgtgcctaatacatgcaagtcgagcgnnncncttcggnggnnagcggcggacgggtgagnaacacgtgggnaatcngccnnncngncnggaataacncnnggaaacgggngctaangccggatannnnnncgngnaggcatctnctngnnnngnaagnnncaantgnnncgcnganngaggagcccgcggcgcattagctngttggngnggtaacggcncaccaaggcgacgatgcgtagccgacctgagagggtgnncggccacactgggactgagacacggcccagactcctacgggaggcagcagtagggaatcttccgcaatgggcgcaagcctgacggagcaacgccgcgtgagcgaagaaggccttcgggttgtaaagctcngtnnctcgggnagagcgncanggngnntggaaagcnccntgngagacggtaccgagngaggaagccccggctaactacgtgccagcagccgcggtaanacgtagggggcnagcgttgtccggaatcactgggncgtaaagngtgcgtangcggtngngnnngtcngnnntgaaagtccanggctcnaccntgggnnngcnttggaaactgcntnnacttgagtgctggagaggcnaggnnaattccncgtgtnancggtgnaantgcgnnananatgnggaggaataccagtggcnaangcgccttngctggacagtgnactgacgctganggcacgaaanncgtggggancaannnnnnnnnnnnnnnnnnnnnnnnnnnnnnnnnacaggattagatncccnnngtagtccnncnccgtaancnatgagtnctnagntgttgggggnnnncacccncantgcngnnggaaanccaataagcactccgcctggggagtncggtcncaagactgaanctcaaaggaattgacgggggcccgcacaagcagtggagcatntggtttaantcgaagcaacgcgaagaaccttancagggctngacatcccnctgacnnnnncagagatgnnnnntcccttcggggcagnggagacaggtggtgcatggttgtcgtcagctcgtgtcgtgagatgttgggttnagtcccgcaacgagcgcaacccttganctgtgttaccagcncgtnnnggnggggactcacagntgactgccggcgtaagtcggaggaaggcggggatgacgtcaaatcatcatgcccntnatgtcctgggctacacacgtgctacaatgggcggnacaangggangcgannccgcgaggnggagcnaanccnnnaaagccgntcgtagttcggattgcaggctgcaactcgcctgcatgaagccggaattgctagtaatcgcggatcagcatgccgcggtgaatncgttcccgggccttgtacacaccgcccgtcacaccacgagagtcggcaacacccgaagtcggtgnggtaaccnntnnnnggagccagccgccgaaggtggggtngatgattggggtgaagtcgtaacaaggtagccgt

SEQ ID NO: 78 - DNA, 1498 bp, Artificial Sequence
  Description of Artificial Sequence Figure 1 majority sequence
agagtttgatcctggctcaggacgaacgctggcggcgtgcctaatacatgcaagtcgagcgggcccttcggggccagcggcggacgggtgagtaacacgtgggtaatctgcctttcagaccggaataacgcccggaaacgggtgctaatgccggatangcacgcgagnaggcatctncttgcggggaaaggtgcaantgcatcgctgagagaggagcccgcggcgcattagctagttggtggggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcaaggggagtggaaagccccttgngagacggtaccgagtgaggaagccccggctaactacgtgccagcagccgcggtaatacgtagggggcaagcgttgtccggaatcactgggcgtaaagcgtgcgtaggcggttgngtaagtctggagtgaaagtccanggctcaaccntgggaatgctttggaaactgcntgacttgagtgctggagaggcaaggggaattccncgtgtagcggtgnaatgcgtagatatgtggaggaataccagtggcgaangcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggctngacatccctctgacagccgcagagatgnggnttcccttcggggcagaggagacaggtggtgcatggttgtcgtcagctcgtgtcgtgagatgttgggttaagtcccgcaacgagcgcaacccttganctgtgttaccagcacgttgaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcaaaacccaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccntntngggagccagccgccgaaggtggggttgatgattggggtgaagtcgtaacaaggtagccgt

SEQ ID NO: 79 - DNA, 718 bp, Alicyclobacillus acidocaldarius
gggggttggatgttacaggcttccatctcgcccgtgtgggacacgggtctcgccgtgctcgcgctgcgcgctgcggggcttccggccgatcactgaccggttggtcaaggctgggctgaatggctgttggaccggcagatcaccgtgccgggcgattgggtggtgaagcgcccgaacctcaacccgggcggcttcgcgctccagttcgacaacgtgtactatccggacgtggacgacacggccgtcgtcatctgggcgctcaacacgctgcgactcccggacgagcgccgcaggcgagacgccatgacgaagggattccggccatgacgaagggattccgctggattgtcggcatgcagagctcgaacggcggctggggcgcatacgacgtcgacaacacgagcgatctcccgaaccacatcccgttctgcgacttcggcgaagtgaccgatccgccgtcggaagacgtcaccgcccacgtgctcgagtgtttcggcagcttcggctacgacgacgcctggaaggtgatccagcgcgcggtggcgtacctcaagcgggagcagaagccggacggcagctggttcggtcgctggggcgtcaactacgtgtatggcatcggcgcggtggtgtcggcgctgaaggcggtcgggatcgacatgcgcgagccgtacattcaaaaggcgctcgattgggtggagcagcatcagaacccggacgg

SEQ ID NO: 80 - DNA, 878 bp, Alicyclobacillus acidocaldarius
ggaggatggatgtttcaggcttccatctcgccggtgtgggacacgggcctcgccgtgctcgcgctgcgcgctgcggggcttccggccgatcacgaccgcttggtcaaggcgggcgagtggctgttggaccggcagatcacggttccgggcgactgggcggtgaagcgcccgaacctcaagccgggcgggttcgcgttccagttcgacaacgtgtactacccggacgtggacgacacggccgtcgtggtgtgggcgctcaacaccctgcgcttgccggacgagcgccgcaggcgggacgccatgacgaagggattccgctggattgtcggcatgcagagctcgaacggcggttggggcgcctacgacgtcgacaacacgagcgatctcccgaaccacatcccgttctgcgacttcggcgaagtgaccgatccgccgtcagaggacgtcaccgcccacgtgctcgagtgtttcggcagcttcgggtacgatgacgcctggaaggtcatccggcgcgcggtggaatatctcaagcgggagcagaagccggacggcagctggttcggtcgttggggcgtcaattacctctacggcacgggcgcggtggtgtcggcgctgaaggcggtcgggatcgacacgcgcgagccgtacattcaaaaggcgctcgactgggtcgagcagcatcagaacccggacggcggctggggcgaggactgccgctcgtacgaggatccggcgtacgcgggtaagggcgcgagcaccccgtcgcagacggcctgggcgctgatggcgctcatcgcgggcggcagggcggagtccgaggccgcgcgccgcggcgtgcaatacctcgtggagacgcagcgcccggacggcggctggga

SEQ ID NO: 81 - DNA, 878 bp, Alicyclobacillus acidoterrestris
gggggttggatgttccaggcgagtatttctccaatctgggatactggcttgaccgtcttggcactgcgttcggctggattgccaccagatcatccagcgctgattaaagcgggtgagtggttggtcagtaaacaaattctcaaggatggcgactggaaagttcgtcgacgcaaggcgaaaccaggcggttgggcatttgaattccactgcgaaaactacccagacgtcgacgatacggcgatggtcgtcttggcgctcaatggcattcaattgccggatgaagggaagcgtcgtgacgcattgacccgtggcttccgttggttgcgcgagatgcagagttcgaacgggggctggggcgcatacgatgtggacaacacgcgtcagttgaccaatcggattccattttgcaacttcggcgaagtgattgatccgccatcggaagacgtcaccgcacacgtcttggagtgcttcggcagctttgggtacgacgaggcatggaaggtgattcgcaaggcggtcgagtatctcaaggcgcaacaacgcccagatgggtcatggtttggccgctggggcgtcaactacgtgtatggcatcggcgcggtcgttccgggactcaaggccgtcggtgtcgatatgcgtgagccgtgggtgcaaaagtcgctcgactggctcgtcgagcatcaaaatgaggatggcggctggggtgaaagccgaattccagcacactggcggccgttactagtggatccgagctcggtaccaagcttggcgtaatcatggtcatagctgtttcctgtgtgaaattggtatccgctcacaattcacacaacatacgagccggaacataagtgtaagcctggggtgcctatgagtgagct

SEQ ID NO: 82 - DNA, 878 bp, Alicyclobacillus acidocaldarius
gggggttggatgttccaggcgagtatttctccaatctgggatactggcttgaccgtcttggcactgcgttcggctggattgccaccagatcatccagcgctgattaaagcgggtgagtggttggtcagtaaacaaattctcaaggatggcgactggaaagttcgtcgacgcaaggcgaaaccaggcggttgggcatttgaattccactgcgaaaactacccagacgtcgacgatacggcgatggtcgtcttggcgctcaatggcattcaattgccggatgaagggaagcgtcgtgacgcattgacccgtggcttccgttggttgcgcgagatgcagagttcgaacgggggctggggcgcatacgatgtggacaacacgcgtcagttgaccaaatcggattccatttttgcgacttcgggcgaagtgattgatccgccatcggaagacgtcaccgcacacgtcttggagtgcttcggcagctttgggtacgacgaggcatggaaggtgattcgcaaggcggtcgagtatctcaaggcgcaacaacgcccagatgggtcatggtttggccgctggggcgtcaactacgtgtatggcatcggcgcggtcgttccgggactcaaggccgtcggtgtcgatatgcgtgagccgtgggtgcaaaagtcgctcgactggctcgtcgagcatcaaaatgaggatggcggttggggtgaagattgccgttcctatgatgatccacgtctcgcaggtcagggtgtgagtacaccgtcgcagaccgcctgggcgttgatggcgctcatcgcgggcggccgtgtcgagtcagatgcggtattgcgcggggtcacttaccttcacgacacgcagcgcgcagatggtggctg

SEQ ID NO: 83 - protein, 631 aa, Alicyclobacillus acidocaldarius
MAEQLVEAPAYARTLDRAVEYLLSCQKDEGYWWGPLLSNVTMEAEYVLLCHILDRVDRDRMEKIRRYLLHEQREDGTWALYPGGPPDLDTTIEAYVALKYIGMSRDEEPMQKALRFIQSQGGIESSRVFTRMWLALVGEYPWEKVPMVPPEIMFLGKRMPLNIYEFGSWARATVVALSIVMSRQPVFPLPERARVPELYETDVPPRRRGAKGGGGWIFDALDRALHGYQKLSVHPFRRAAEIRALDWLLERQAGDGSWGGIQPPWFYALIALKILDMTQHPAFIKGWEGLELYGVELDYGGWMFQASISPVWDTGLAVLALRAAGLPADHDRLVKAGEWLLDRQITVPGDWAVKRPNLKPGGFAFQFDNVYYPDVDDTAVVVWALNTLRLPDERRRRDAMTKGFRWIVGMQSSNGGWGAYDVDNTSDLPNHIPFCDFGEVTDPPSEDVTAHVLECFGSFGYDDAWKVIRRAVEYLKREQKPDGSWFGRWGVNYLYGTGAVVSALKAVGIDTREPYIQKALDWVEQHQNPDGGWGEDCRSYEDPAYAGKGASTPSQTAWALMALIAGGRAESEAARRGVQYLVETQRPDGGWDEPYYTGTASPGDFYLGYTMYRHVFPTLALGRYKQAIERR

SEQ ID NO: 84 - protein, 631 aa, Alicyclobacillus acidocaldarius [sequence omitted]

SEQ ID NO: 85 - protein, 634 aa, Alicyclobacillus acidoterrestris
MTKQLLDTPMVQATLEAGVAHLLRRQAPDGYWWAPLLSNVCMEAEYVLLCHCLGKKNPEREAQIRKYIISQRREDGTWSIYPGGPSDLNATVEAYVALKYLGEPASDPQMVQAKEFIQNEGGIESTRVFTRLWLAMVGQYPWDKLPVIPPEIMHLPKSVPLNIYDFASWARATIVTLSYRHESPTCDATSGLCKGSGIVRGEGPPKRRSAKGGDSGFFVALDKFLKAYNKWPIQPGRKSGEQKALEWILAHQEADGCWGGIQPPWFYALLALKCLNMTDHPAFVKGFEGLEAYGVHTSDGGWMFQASISPIWDTGLTVLALRSAGLPPDHPALIKAGEWLVSKQILKDGDWKVRRRKAKPGGWAFEFHCENYPDVDDTAMVVLALNGIQLPDEGKRRDALTRGFRWLREMQSSNGGWGAYDVDNTRQLTKSDSIFATSGEVIDPPSEDVTAHVLECFGSFGYDEAWKVIRKAVEYLKAQQRPDGSWFGRWGVNYVYGIGAVVPGLKAVGVDMREPWVQKSLDWLVEHQNEDGGWGEDCRSYDDPRLAGQGVSTPSQTAWALMALIAGGRVESDAVLRGVTYLHDTQRADGGWDEEVYTGTGFPGDFYLAYTMYRDILPVWALGRYQEAMQRIRG

SEQ ID NO: 86 - protein, 556 aa, Bacillus subtilis
MGTLQEKVRRFQKKTITELRDRQNADGSWTFCFEGPIMTNSFFILLLTSLDEGENEKELISSLAAGIHAKQQPDGTFINYPDETRGNLTATVQGYVGMLASGCFHRTEPHMKKAEQFIISHGGLRHVHFMTKWMLAANGLYPWPALYLPLSLMALPPTLPIHFYQFSSYARIHFAPMAVTLNQRFVLINRNISSLHHLDPHMTKNPFTWLRSDAFEERDLTSILLHWKRVFHAPFAFQQLGLQTAKTYMLDRIEKDGTLYSYASATIYMVYSLLSLGVSRYSPIIRRAITGIKSLVTKCNGIPYLENSTSTVWDTALISYALQKNGVTETDGSVTKAADFLLERQHTKIADWSVKNPNSVPGGWGFSNINTNNPDCDDTTAVLKAIPRNHSPAAWERGVSWLLSMQNNDGGFSAFEKNVNHPLIRLLPLESAEDAAVDPSTADLTGRVLHFLGEKVGFTEKHQHIQRAVKWLFEHQEQNGSWYGRWGVCYIYGTWAALTGMHACGLTESIPVYKRLCVGSNPYKMMTEAGENPAKAPKSKHMYRFIEEPLYKRPGL

SEQ ID NO: 87 - protein, 706 aa, Dictyostelium discoideum
FTRMTTTNWSLKVDRGRQTWEYSQEKKEATDVDIHLLRLKEPGTHCPEGCDLNRAKTPQQAIKKAFQYFSKVQTEDGHWAGDYGGPMFLLPGLVITCYVTGYQLPESTQREIIRYLFNRQNPVDGGWGLHIEAHSDIFGTTLQYVSLRLLGVPADHPSVVKARTFLLQNGGATGIPSWGKFWLATLNAYDWNGLNPIPIEFWLLPYNLPIAPGRWWCHCRMVYLPMSYIYAKKTTGPLTDLVKDLRREIYCQEYEKINWSEQRNNISKLDMYYEHTSLLNVINGSLNAYEKVHSKWLRDKAIDYTFDHIRYEDEQTKYIDIGPVNKTVNMLCVWDREGKSPAFYKHADRLKDYLWLSFDGMKMQGYNGSQLWDTAFTIQAFMESGIANQFQDCMKLAGHYLDISQVPEDARDMKHYHRHYSKGAWPFSTVDHGWPISDCTAEGIKSALALRSLPFIEPISLDRIADGINVLLTLQNGDGGWASYENTRGPKWLEKFNPSEVFQNIMIDYSYVECSAACIQAMSAFRKHAPNHPRIKEINRSIARGVKFIKSIQRQDGSWLGSWGICFTYGTWFGIEGLVASGEPLTSPSIVKACKFLASKQRADGGWGESFKSNVTKEYVQHETSQVVNTGWALLSLMSAKYPDRECIERGIKFLIQRQYPNGDFPQESIIGVFNFNCMISYSNYKNIFPLWALSRYNQLYLKSKI

SEQ ID NO: 88 - protein, 647 aa, Synechocystis PCC6803
MVIAASPSVPCPSTEQVRQAIAASRDFLLSEQYADGYWWSELESNVTITAEVVILHKIWGTAAQRPLEKAKNYLLQQQRDHGGWELYYGDGGELSTSVEAYTALRILGVPATDPALVKAKNFIVGRGGISKSRIFTKMHLALIGCYDWRGTPSIPPWVMLLPNNFFFNIYEMSSWARSSTVPLMIVCDQKPVYDIAQGLRVDELYAEGMENVQYKLPESGTIWDIFIGLDSLFKLQEQAKVVPFREQGLALAEKWILERQEVSGDWGGIIPAMLNSLLALKVLGYDVNDLYVQRGLAAIDNFAVETEDSYAIQACVSPVWDTAWVVRALAEADLGKDHPALVKAGQWLLDKQILTYGDWQIKNPHGEPGAWAFEFDNNFYPDIDDTCVVMMALQGITLPDEERKQGAINKALQWIATMQCKTGGWAAFDIDNDQDWLNQLPYGDLKAMIDPSTADITARVVEMLGACGLTMDSPRVERGLTYLLQEQEQDGSWFGRWGVNYLYGTSGALSALAIYDAQRFAPQIKTAIAWLLSCQNADGGWGETCESYKNKQLKGQGNSTASQTAWALIGLLDALKYLPSLGQDAKLTTAIEGGVAFLVQGQTPKGTWEEAEYTGTGFPCHFYIRYHYYRQYFPLIALARYSHLQAS

SEQ ID NO: 89 - protein, 680 aa, Streptomyces coelicolor
MTATTDGSTGASLRPLAASASDTDITIPAAAAGVPEAAARATRRATDFLLAKQDAEGWWKGDLETNVTMDAEDLLLRQFLGIQDEETTRAAALFIRGEQREDGTWATFYGGPGELSTTIEAYVALRLAGDSPEAPHMARAAEWIRSRGGIASARVFTRIWLALFGWWKWDDLPELPPELIYFPTWVPLNIYDFGCWARQTIVPLTIVSAKRPVRPAPFPLDELHTDPARPNPPRPLAPVASWDGAFQRIDKALHAYRKVAPRRLRRAAMNSAARWIIERQENDGCWGGIQPPAVYSVIALYLLGYDLEHPVMRAGLESLDRFAVWREDGARMIEACQSPVWDTCLATIALADAGVPEDHPQLVKASDWMLGEQIVRPGDWSVKRPGLPPGGWAFEFHNDNYPDIDDTAEVVLALRRVRHHDPERVEKAIGRGVRWNLGMQSKNGAWGAFDVDNTSAFPNRLPFCDFGEVIDPPSADVTAHVVEMLAVEGLAHDPRTRRGIQWLLDAQETDGSWFGRWGVNYVYGTGSVIPALTAAGLPTSHPAIRRAVRWLESVQNEDGGWGEDLRSYRYVREWSGRGASTASQTGWALMALLAAGERDSKAVERGVAWLAATQREDGSWDEPYFTGTGFPWDFSINYNLYRQVFPLTALGRYVHGEPFAKKPRAADAPAEAAPAEVKGS

SEQ ID NO: 90 - protein, 741 aa, Artificial Sequence
  Description of Artificial Sequence Figure 6 majority sequence
MTXXXXXXXXXXXEQLVEAXXXXXXXXXPXXXXXXXXXXXXXXXXXXXXXXXXXXEAVARALDRAVDYLLSRQKADGYWWGPLLSNVTMEAEYVLLCHILGRVDRERXXMEKIRRYLLHEQREDGTWALYPGGPXGDLSTTVEAYVALKYLGXVSADEPHMVKALEFIQSQGGIESSRVFTRMWLALVGEYPWDKLPMIPPEIMLLPKNVPLNIYEFGSWARATVVPLSIVMAQQPVXXXXFPLPELARVPELYETDVPPRRXRGAKGGGGWXXXIFDAXXLDSALHGYQKAXXAVHPFRRAGEARALTWILERQEGDGSWGGIQPPWFYALIALKVLGMTXQHPAFIKGLEGLELYGVELSDGGWMFQAXSISPVWDTGLAVLALRAAGLPADHPALVKAGEWLLDRQITVPGDWAVKRXXPNLKPGGWAFEFDNVNYPDVDDTAVVVXXXLALNGLRLPDEERRRDAITKGFRWLLGMQSSNGGWGAYDVDNTSDLPNHLPXFCDFGEVXIDPPSADVTAHVLECLGSXXXFGXXXXXYDEAWKVIRRAVEYLKREQEQDGSWFGRWGVNYLYGTGAVVSALKAVGLDTREPYIQKALDWLESHQNADGGWGEDCRSYEXDPEYAGQGASTASQTAWALMALIAGXXXXXXXGRAEXXSEAAERGVAYLVETQRPDGGWDEPYYTGTGFPGDFYLGYTMYRQVFPLLALGRYKQAXXXXXXXXXXXXXXXXXXXERXGS

SEQ ID NO: 91 - DNA, 376 bp, Zygosaccharomyces bailii
tgcatggccgttcttagttggtggagtgatttgtctgcttaattgcgataacgaacgagaccttaacctactaaatagtaggtgctagcatttgctggtttttccacttcttagagggactatcggtttcaagccgatggaagtttgaggcaataacaggtctgtgatgcccttagacgttctgggccgcacgcgcgctacactgacggagccagcgagtctaaccttggccgagaggtctgggtaatcttgtgaaactccgtcgtgctggggatagagcattgtaattattgctcttcaacgaggaattcctagtaagcgcaagtcatcaacttgcgttgattacgtccctgccctttgtacacacaagccgaat

SEQ ID NO: 92 - DNA, 404 bp, Saccharomyces humaticus
ctctttcttgattttgtgggtggtggtgcatggccgttcttagttggtggagtgatttgtctgcttaattgcgataacgaacgagaccttaacctactaaatagtggtgctagcatttgctggttatccacttcttagagggactatcggtttcaagccgatggaagtttgaggcaataacaggtctgtgatgcccttagacgttctgggccgcacgcgcgctacactgacggagccagcgagtctaaccttggccgagaggtcttggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctag

SEQ ID NO: 93 - DNA, 408 bp, Candida colliculosa
ctctttcttgattttgtgggtggtggtgcatggccgttcttagttggtggagtgatttgtctgcttaattgcgataacgaacgagaccttaacctactaaatagtggtgctagcatttgctggttatccacttcttagagggactatcggtttcaagccgatggaagtttgaggcaataacaggtctgtgatgcccttagacgttctgggccgcacgcgcgctacactgacggagccagcgagtctaaccttggccgagaggtctgggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctagtacc

SEQ ID NO: 94 - DNA, 303 bp, Vitis vinifera
ctctttcttgattctatgggtggtggtgcatggccgttcttagttggtggagcgatttgtctggttaattccgttaacgaacgagacctcagcctgctaactagctatgtgaaggtgagcctccgcagccagcttcttagagggactatggccgcttaggccaaggaagtttgaggcaataacaggtctgtgatgcccttagatgttctgggccgcacgcgcgctacactgatgtattcaacgagtctatagccttggccgacaggcccgggtaatctttgaaatttcatcgtgatggggata

SEQ ID NO: 95 - DNA, 407 bp, Zygosaccharomyces rouxii
ctctttcttgattttgtgggtggtggtgcatggccgttcttagttggtggagtgatttgtctgcttaattgcgataacgaacgagaccttaacctactaaatagtggtgctagcatttgctggtttttccacttcttagagggactatcggtttcaagccgatggaagtttgaggcaataacaggtctgtgatgcccttagacgttctgggccgcacgcgcgctacactgacggagccaacgagtctaaccttggccgagaggtctgggtaatcttgtgaaactccgtcgtgctggggatagagcattgtaattattgctcttcaacgaggaattcctagtaagcgcaagtcatcagcttgcgttgattacgtccctgccctttgtacacaccgcccgtcgctagta

SEQ ID NO: 96 - DNA, 393 bp, Penicillium digitatum
gtgctggaattcggctttgcatggccgttcttagttggtggagtgatttgtctgcttaattgcgataacgaacgagacctcggcccttaaatagcccggtccgcatttgcgggccgctggcttcttaaggggactatcggctcaagccgatggaagtgcgcggcaataacaggtctgtgatgcccttagatgttctgggccgcacgcgcgctacactgacagggccagcgagtacatcaccttaaccgagaggtttgggtaatcttgttaaaccctgtcgtgctggggatagagcattgcaattattgctcttcaacgaggaatgcctagtaggcacgagtcatcagctcgtgccgattacgtccctgccctttgtacacacaagccgaattc

SEQ ID NO: 97 - DNA, 400 bp, Byssochlamys fulva
tgctggaattcggctttgcatggccgttcttagttggtggagtgatttgtctgcttaattgcgataacgaacgagacctcggctcttaaatagcccggtccgcgtttgcgggccgctggcttcttagggggactatcggctcaagccgatggaagtgcgcggcaataacaggtctgtaatgcccttagatgttctgggccgcacgcgcgctacactgacagggccagcgggtacatcaccttggccgagaggtctgggtaatcttgttaaaccctgtcgtgctggggatagagcattgcaattattgctcttcaacgaggaatgcctagtaggcacgagtcatcagctcgtgccgattacgtccctgccctttgtacacacaagccgaattctgcagata

SEQ ID NO: 98 - DNA, 416 bp, Penicillium chrysogenum
tctttcttgatcttttggatggtggtgcatggccgttcttagttggtggagtgatttgtctgcttaattgcgataacgaacgagacctcggcccttaaatagcccggtccgcatttgcgggccgctggcttcttagggggactatcggctcaagccgatggaagtgcgcggcaataacaggtctgtgatgcccttagatgttctgggccgcacgcgcgctacactgacagggccagcgagtacatcaccttaaccgagaggtttgggtaatcttgttaaaccctgtcgtgctggggatagagcattgcaattattgctcttcaacgaggaatgcctagtaggcacgagtcatcagctcgtgccgattacgtccctgccctttgtacacaccgcccgtcgctactaccgattgaatg

SEQ ID NO: 99 - DNA, 406 bp, Aspergillus nidulans
agctctttcttgatcttttggatggtggtgcatggccgttcttagttggtggagtgatttgtctgcttaattgcgataacgaacgagacctcggcccttaaatagcccggtccgcgtccgcgggccgctggcttcttagggggactatcggctcaagccgatggaagtgcgcggcaataacaggtctgtgatgcccttagatgttctgggccgcacgcgcgctacactgacagggccagcgagtacatcaccttggccgagaggcccgggtaatcttgttaaaccctgtcgtgctggggatagagcattgcaattattgctcttcaacgaggaatgcctagtaggcacgagtcatcagctcgtgccgattacgtccctgccctttgtacacaccgcccgtcgctac

SEQ ID NO: 100 - DNA, 427 bp, Eurotium amstelodami
tttcttgatcttttggatggtggtgcatggccgttcttagttggtggagtgatttgtctgcttaattgcgataacgaacgagacctcggcccttaaatagcccggtccgcatttgcgggccgctggcttcttagggggactatcggctcaagccgatggaagtgcgcggcaataacaggtctgtgatgcccttagatgttctgggccgcacgcgcgctacactgacagggccagcgagtacatcaccttaaccgagaggtctgggtaatcttgttaaaccctgtcgtgctggggatagagcattgcaattattgctcttcaacgaggaatgcctagtaggcacgagtcatcagctcgtgccgattacgtccctgccctttgtacacaccgcccgtcgctactaccgattgaatggctcggtgaggcc

SEQ ID NO: 101 - DNA, 442 bp, Aspergillus candidus
ctctttcttgatcttttggatggtggtgcatggccgttcttagttggtggagtgatttgtctgcttaattgcgataacgaacgagacctcggcccttaaatagcccggtccgcatttgcgggccgctggcttcttagggggactatcggctcaagccgatggaagtgcgcggcaataacaggtctgtgatgcccttagatgttctgggccgcacgcgcgctacactgacagggccagcgagtacatcaccttggccgagaggtctgggtaatcttgttaaaccctgtcgtgctggggatagagcattgcaattattgctcttcaacgaggaatgcctagtaggcacgagtcatcagctcgtgccgattacgtccctgccctttgtacacaccgcccgtcgctactaccgattgaatggctcggtgaggcctccggactggct

SEQ ID NO: 102 - DNA, 407 bp, Gallus gallus
ctctttctcgattccgtgggtggtggtgcatggccgttcttagttggtggagcgatttgtctggttaattccgataacgaacgagactctggcatgctaactagttacgcgacccccgagcggtcggcgtccaacttcttagagggacaagtggcgttcagccacccgagattgagcaataacaggtctgtgatgcccttagatgtccggggctgcacgcgcgctacactgactggctcagcttgtgtctaccctacgccggcaggcgcgggtaacccgttgaaccccattcgtgatggggatcggggattgcaattattccccatgaacgaggaattcccagtaagtgcgggtcataagctcgcgttgattaagtccctgccctttgtacacaccgcccgtcgcta

SEQ ID NO: 103 - DNA, 407 bp, Triticum aestivum
ctctttcttgattctatgggtggtggtgcatggccgttcttagttggtggagcgatttgtctggttaattccgttaacgaacgagacctcagcctgctaactagctatgcggagccatccctccgcagctagcttcttagagggactatcgccgtttaggcgacggaagtttgaggcaataacaggtctgtgatgcccttagatgttctgggccgcacgcgcgctacactgatgtattcaacgagtatatagccttggccgacaggcccgggtaatcttgggaaatttcatcgtgatggggatagatcattgcaattgttggtcttcaacgaggaatgcctagtaagcgcgagtcatcagctcgcgttgactacgtccctgccctttgtacacaccgcccgtcgctc

SEQ ID NO: 104 - DNA, 411 bp, Artificial Sequence
  Description of Artificial Sequence Figure 7 consensus sequence
ctctttcttgattttttgggtggtggtgcatggccgttcttagttggtggagtgatttgtctgcttaattgcgataacgaacgagacctcggcctnctaaatagcncctgtccgnncnnatttgcgggccngctggcttcttagagggactatcggcntcaagccgatggaagtttgcggcaataacaggtctgtgatgcccttagatgttctgggccgcacgcgcgctacactgacggggccagcgagtacataaccttggccgagaggtctgggtaatcttgtgaaaccctgtcgtgctggggatagagcattgcaattattgctcttcaacgaggaatgcctagtaggcgcgagtcatcagctcgtgttgattacgtccctgccctttgtacacaccgcccgtcgcta

SEQ ID NO: 105 - DNA, 19 bp, Artificial Sequence
  Description of Artificial Sequence Synthetic oligonucleotide primer
gtggtgctagcatttgctg

SEQ ID NO: 106 - DNA, 17 bp, Artificial Sequence
  Description of Artificial Sequence Synthetic oligonucleotide primer
ccgctggcttcttaggg

SEQ ID NO: 107 - DNA, 18 bp, Artificial Sequence
  Description of Artificial Sequence Synthetic oligonucleotide primer
ggagccagcgagtctaac

SEQ ID NO: 108 - DNA, 19 bp, Artificial Sequence
  Description of Artificial Sequence Synthetic oligonucleotide primer
agggccagcgagtacatca

SEQ ID NO: 109 - DNA, 21 bp, Artificial Sequence
  Description of Artificial Sequence Synthetic oligonucleotide probe
cggtttcaagccgatggaagt

SEQ ID NO: 110 - DNA, 20 bp, Artificial Sequence
  Description of Artificial Sequence Synthetic oligonucleotide probe
ctcaagccgatggaagtgcg

SEQ ID NO: 111 - DNA, 1500 bp, Alicyclobacillus acidocaldarius; identical to SEQ ID NO: 74

SEQ ID NO: 112 - DNA, 1520 bp, Alicyclobacillus acidocaldarius
  modified_base (236): a, t, c or g
agagtttgatcctggctcaggacgaacgctggcggcgtgcctaatacatgcaagtcgagcgggtctcttcggaggccagcggcggacgggtgaggaacacgtgggtaatctgcctttcaggccggaataacgcccggaaacgggcgctaatgccggatacgcccgcgaggaggcatcttcttgcgggggaaggcccaattgggccgctgagagaggagcccgcggcgcattagctngttggcggggtaacggcccaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcggcatgggggatggaaagccccntgcgagacggtaccgagtgaggaagccccggctaactacgtgccagcagccgcggtaaaacgtagggggcgagcgttgtccggaatcactgggcgtaaagggtgcgtaggcggtcgagcaagtctggagtgaaagtccatggctcaaccatgggatggctttggaaactgcttgacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgacaccctcagagatgaggggtcccttcggggcagaggagacaggtggtgcatggttgtcgtcagctcgtgtcgtgagatgttgggttcagtcccgcaacgagcgcaacccttgacctgtgttaccagcgcgttgaggcggggactcacaggtgactgccggcgtaagtcggaggaaggcggggatgacgtcaaatcatcatgcccctgatgtcctgggctacacacgtgctacaatgggcggaacaaagggaggcgaagccgcgaggcggagcgaaacccaaaaagccgctcgtagttcggattgcaggctgcaactcgcctgcatgaagccggaattgctagtaatcgcggatcagcatgccgcggtgaatacgttcccgggccttgtacacaccgcccgtcacaccacgagagtcggcaacacccgaagtcggtgaggtaacccctgtggggagccagccgccgaaggtggggtcgatgattggggtgaagtcgtaacaaggtagccgtaccggaaggtgcggttggat

SEQ ID NO: 113 - DNA, 1497 bp, Alicyclobacillus cycloheptanicus
agagtttgatcctggctcaggacgaacgctggcggcgtgcctaatacatgcaagtcgagcggacccttcggggtcagcggcggacgggtgagtaacacgtgggtaatctgcccaactgaccggaataacgcctggaaacgggtgctaatgccggataggcagcgagcaggcatctgctcgctgggaaaggtgcaaatgcaccgcagatggaggagcccgcggcgcattagctggttggtggggtaacggctcaccaaggcgacgatgcgtagccgacctgagagggtggacggccacactgggactgagacacggcccagactcctacgggaggcagcagtagggaatcttccgcaatgggcgcaagcctgacggagcaacgccgcgtgagcgaagaaggccttcgggttgtaaagctcagtcactcgggaagagcggcaaggggagtggaaagccccttgagagacggtaccgagagaggaagccccggctaactacgtgccagcagccgcggtaatacgtagggggcaagcgttgtccggaatcactgggcgtaaagcgtgcgtaggcggttgcgtgtgtccggggtgaaagtccagggctcaaccctgggaatgccttggaaactgcgtaacttgagtgctggagaggcaaggggaattccgcgtgtagcggtggaatgcgtagatatgcggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccccctgacagccgcagagatgcggtttcccttcggggcaggggagacaggtggtgcatggttgtcgtcagctcgtgtcgtgagatgttgggttaagtcccgcaacgagcgcaacccttgaactgtgttaccagcacgtgaaggtggggactcacagttgactgccggcgtaagtcggaggaaggcggggatgacgtcaaatcatcatgccctttatgtcctgggctacacacgtgctacaatgggcggtacaacgggaagcgagaccgcgaggtggagcaaacccctgaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtggggtaacccgtcagggggccagccgccgaaggtggggttgatgattggggtgaagtcgtaacaaggtagccgt

SEQ ID NO: 114 - DNA, 1517 bp, Alicyclobacillus cycloheptanicus
agagtttgatcctggctcaggacgaacgctggcggcgtgcctaatacatgcaagtcgagcggacccttcggggtcagcggcggacgggtgagtaacacgtgggtaatctgcccaactgaccggaataacgcctggaaacgggtgctaatgccggataggcagcgagcaggcatctgctcgctgggaaaggtgcaaatgcaccgcagatggaggagcccgcggcgcattagctggttggtggggtaacggctcaccaaggcgacgatgcgtagccgacctgagagggtggacggccacactgggactgagacacggcccagactcctacgggaggcagcagtagggaatcttccgcaatgggcgcaagcctgacggagcaacgccgcgtgagcgaagaaggccttcgggttgtaaagctcagtcactcgggaagagcggcaaggggagtggaaagccccttgagagacggtaccgagagaggaagccccggctaactacgtgccagcagccgcggtaatacgtagggggcaagcgttgtccggaatcactgggcgtaaagcgtgcgtaggcggttgcgtgtgtccggggtgaaagtccagggctcaaccctgggaatgccttggaaactgcgtaacttgagtgctggagaggcaaggggaattccgcgtgtagcggtggaatgcgtagatatgcggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccccctgacagccgcagagatgcggtttcccttcggggcaggggagacaggtggtgcatggttgtcgtcagctcgtgtcgtgagatgttgggttaagtcccgcaacgagcgcaacccttgaactgtgttaccagcacgtgaaggtggggactcacagttgactgccggcgtaagtcggaggaaggcggggatgacgtcaaatcatcatgccctttatgtcctgggctacacacgtgctacaatgggcggtacaacgggaagcgagaccgcgaggtggagcaaacccctgaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtggggtaacccgtcagggggccagccgccgaaggtggggttgatgattggggtgaagtcgtaacaaggtagccgtatcggaaggtgcggttggat

SEQ ID NO: 115 - DNA, 770 bp, Alicyclobacillus acidoterrestris
  modified_base (549)..(549): a, c, g, or t
gacgaacgctggcggcgtgcctaatacatgcaagtcgagcgagcccttcggggctagcggcggacgggtgagtaacacgtgggcaatccgcctttcagactggaataacactcggaaacgggtgctaatgccggataatacacgggtaggcatctacttgtgttgaaagatgcaactgcatcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactggggcgtaaagcgtgcgtangcggttgtgtaagtctgaactgaaagtccaaggctcnaccttgggnatgctttggaaactgcatggacttgagtgctggagaggcnaggcnaattccncgtgttaccggtgnaaatgcgntanatatgtggaggaataccagtggcnaangcgcctttgctggacagtggactgacgctgaaggcacgaaaancgtggggancaacnggattanatccccn

SEQ ID NO: 116 - DNA, 1514 bp, Alicyclobacillus acidoterrestris
agagtttgatcctggctcaggacgaacgctggcggcgtgcctaatacatgcaagtcgagcgagcccttcggggctagcggcggacgggtgagtaacacgtgggcaatctgcctttcagactggaataacactcggaaacgggtgctaatgccggataatacacgggtaggcatctacttgtgttgaaagatgcaactgcatcgctgagagaggagcccgcggcgcattagctagttggtgaggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcggggagagcgacaaggagagtggaaagctccttgtgagacggtaccgagtgaggaagccccggctaactacgtgccagcagccgcggtaatacgtagggggcaagcgttgtccggaatcactgggcgtaaagcgtgcgtaggcggttgtgtaagtctgaagtgaaagtccaaggctcaaccttgggattgctttggaaactgcatgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggacacaccccagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatccctctgaccggtgcagagatgtaccttcccttcggggcagaggagacaggtggtgcatggttgtcgtcagctcgtgtcgtgagatgttgggttaagtcccgcaacgagcgcaacccttgatctgtgttaccagcacgtagaggtggggactcacaggtgactgccggcgtaagtcggaggaaggcggggatgacgtcaaatcatcatgccctttatgtcctgggctacacacgtgctacaatgggcggtacaacgggaagcgaagccgcgaggtggagcaaaacctaaaaagccgttcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaaccgttatggagccagccgccgaaggtggggttgatgattggggtgaagtcgtaacaaggtagccgtatcggaaggtgcggttgga

SEQ ID NO: 117 - DNA, 1492 bp, Clostridium elmenteitii
agagtttgatcctggctcaggatgaacgctggcggcgtgcctaacacatgcaagtcgagcggagtgcctttttggacattttcggatggaagaagaggttacttagcggcggacgggtgagtaacgcgtgggcaaccaaccttgatcagggggacaacattgggaaaccagtgctaataccgcatagctctatattatggcatcatgagatagagaaagatttatcggatcaagacgggcccgcgtctgattagctagttggtaaggtaacggcttaccaaggccttgatcagtagccgacctgagagggtgaccggccacactggaactgagacacggtccagactcctacgggaggcagcagtggggaatattgcacaatgggggaaaccctgatgcagcaacgccgcgtgagcgaagaaggccttcgggtcgtaaagctctgtcctatgggaagaaggagtgacggtaccataggaggaagccccggctaactacgtgccagcagccgcggtaatacgtagggggcaagcgttatccggaatcactgggcgtaaagggtgcgtaggcggctaagtaagtcaggggtgaaaggctacggctcaaccgtagtaagcctttgaaactgcttagcttgagtgcaggagaggtaagtggaattcctagtgtagcggtgaaatgcgtagatattaggaggaacaccagtggcgaaggcgacttactggactgtaactgacgctgaggcacgaaagcgtgggagcgaacaggattagataccctggtagtccacgccgtaaacgatgagtgctaggtgttgggggtcaaacctcagtgccggagcaaacgcaataagcactccgcctggggagtacgctcgcaagagtgaaactcaaaggaattgacgggggacccgcacaagcagcggagcatgtggtttaattcgaagcaacgcgaagaaccttacctgagcttgacatccctctgaccggtgagtaaagtcacctttccttcgggacagaggagacaggtggtgcatggttgtcgtcagctcgtgtcgtgagatgttgggttaagtcccgcaacgagcgcaacccctgtcattagttgccagcatttcggatgggcactctaatgagactgccggtgacaaaccggaggaaggtggggatgacgtcaaatcatcatgccccttatgttcagggctacacacgtgctacaatggccgatacaaagggcagcgaaggagcaatccggagcgaaccccataaagtcggtcccagttcggattgagggctgcaactcgcccccatgaagttggagttgctagtaatcgcgaatcagcatgtcgcggtgaatgcgttcccgggtcttgtacacaccgcccgtcacaccacggaagtcggaagcacccgaagcccgttaccgaaccttcgggacggaacggtcgaaggtgaagccgataactggggtgaagtcgtaacaaggtatccgt

SEQ ID NO: 118 - DNA, 1548 bp, Geobacillus subterraneus
gagtttgatcctggctcaggacgaacgctggcggcgtgcctaatacatgcaagtcgagcggaccgaatgagagcttgctcttatttggtcagcggcggacgggtgagtaacacgtgggcaacctgcccgcaagaccgggataactccgggaaaccggagctaataccggataacaccgaagaccgcatggtcttcggttgaaaggcggcctttggctgtcacttgcggatgggcccgcggcgcattagctagttggtgaggtaacggctcaccaaggcgacgatgcgtagccggcctgagagggtgaccggccacactgggactgagacacggcccagactcctacgggaggcagcagtagggaatcttccgcaatggacgaaagtctgacggagcgacgccgcgtgagcgaagaaggccttcgggtcgtaaagctctgttgtgagggacgaaggagcgccgtttgaacaaggcggcgcggtgacggtacctcacgagaaagccccggctaattacgtgccagcagccgcggtaatacgtagggggcgagcgttgtccggaattattgggcgtaaagcgcgcgcaggcggttccttaagtctgatgtgaaagcccacggctcaaccgtggagggtcattggaaactgggggacttgagtgcaggagaggagagcggaattccacgtgtagcggtgaaatgcgtagagatgtggaggaacaccagtggcgaaggcggctctctggcctgtaactgacgctgaggcgcgaaagcgtggggagcaaacaggattagataccctggtagtccacgccgtaaacgatgagtgctaagtgttagaggggtcacaccctttagtgctgcagctaacgcgataagcactccgcctggggagtacggccgcaaggctgaaactcaaaggaattgacgggggcccgcacaagcggtggagcatgtggtttaattcgaagcaacgcgaagaaccttaccaggtcttgacatcccctgacaacccaagagattgggcgttcccccttcggggggacagggtgacaggtggtgcatggttgtcgtcagctcgtgtcgtgagatgttgggttaagtcccgcaacgagcgcaacccttgcctctagttgccagcattcagttgggcactctagagggactgccggcgaaaagtcggaggaaggtggggatgacgtcaaatcatcatgccccttatgacctgggctacacacgtgctacaatgggcggtacaaagggctgcgaacccgcgagggggagcgaatcccaaaaagccgctctcagttcggattgcaggctgcaactcgcctgcatgaagccggaatcgctagtaatcgcggatcagcatgccgcggtgaatacgttcccgggccttgtacacaccgcccgtcacaccacgagagcttgcaacacccgaagtcggtgaggtaacccttacgggagccagccgccgaaggtggggcaagtgattggggtgaagtcgtaacaaggtagccgtaccggaaggtgcggctggatcacctcct

SEQ ID NO: 119 - DNA, 1496 bp, Sulfobacillus disulfidooxidans
agagtttgatcctggctcaggacgaacgctggcggcgtgcctaatacatgcaagtcgagcggactcctacgggagtgagcggcggacgggtgaggaacacgtgggcaatctgcccattggactggaataacgcctggaaacgggtgctaaggccagatagacacagaagaggcctctcttgtgtgggaaagatgctacggcatcgccagtggaggagcccgcggcgcattagctggttggcggggtaacggaccaccaaggcgacgatgcgtagccgacctgagagggtgaacggccacactgggactgagacacggcccagactcctacgggaggcagcagtagggaatcttccgcaatgggcgcaagcctgacggagcaacgccgcgtaagcgaagaaggccttcgggttgtaaagcttagtcactcgggaagagcgggtgggagagggaatgctcccaccgagacggtaccgggagaggaagccccggcaaactacgtgccagcagccgcggtaatacgtagggggcaagcgttgtccggaatcactgggcgtaaagggtgcgtaggcggtgttgtgggtctgaggtgaaaggtcggggctcaaccctgagaatgccttggaaactgcaagacttgagtgctggagaggcaaggggaattccacgtgtagcggtgaaatgcgtagagatgtggaggaataccagtggcgaaggcgccttgctggacagtgactgacgctgaggcacgaaagcgtggggagcaaacaggattagataccctggtagtccacgccgtaaacgatgagtgctaggtgttggggggtaccaccctcagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatcccccagacgggtgtagagatacaccgtcccttcggggctggggagacaggtggtgcatggttgtcgtcagctcgtgtcgtgagatgttgggttaagtcccgcaacgagcgcaacccttgatcggtgttaccagcgcgtaaaggcggggactcaccggtgactgccgtcgtaagacggaggaaggcggggatgacgtcaaatcatcatgccccttatgtcctgggcgacacacgtgctacaatgggcggcacaacgggacgcgagagagcaatctggagccaacccctgaaaaccgctcgtagttcggattgcaggctgcaactcgcctgcatgaagccggaattgctagtaatcgcggatcagcatgccgcggtgaatccgttcccgggccttgtacacaccgcccgtcacaccacgagagtcgacaacacccgaagtcggtggggtaacccgtaagggggccagccgccgaaggtggggccgatgattggggtgaagtcgtaacaaggtagcc

SEQ ID NO: 120 - DNA, 1428 bp, Bacillus thermoleovorans
  modified_base (901)..(902): a, t, c or g
gagagcttgatcctggctcaggacgaacgctggcggcgtgcctaatacatgcaagtcggaccaaatcggagcttgctctgatttggtcagcggcggacgggtgagtaacacgtgggcaacctgcccgcaagaccgggataactccgggaaaccggagctaataccggataacaccgaagaccgcatggtctttggttgaaaggcggctttggctgtcacttgcggatgggcccgcggcgcattagctagttggtgaggtaacggctcaccaaggcgacgatgcgtagccggcctgagagggtgaccggccacactgggactgagacacggcccagactcctacgggaggcagcagtagggaatcttccgcaatgggcgaaagcctgacggagcgacgccgcgtgagcgaagaaggccttcgggtcgtaaagctctgttgtgagggacgaaggagcgccgttcgaagagggcggcgcggtgacggtacctcacgaggaagccccggctaactacgtgccagcagccgcggtaatacgtagggggcgagcgttgtccggaattattgggcgtaaagcgcgcgcaggcggttccttaagtctgatgtgaaagcccacggctcaaccgtggagggtcattggaaactgggggacttgagtgcaggagaggagagcggaattccacgtgtagcggtgaaatgcgtagagatgtggaggaacaccagtggcgaaggcggctctctggcctgcaactgacgctgaggcgcgaaagctggggagcaaacaggattagataccctggtagtccacgccgtaaacgatgagtgctaagtgttagaggggtcacaccctttagtgctgcagtaacgcgataagcactccgcctggggagtacggccgcaagnntgaaactcaaaggaattgacgggggcccgcacaagcggtggagcatgtggtttaattcgaagcaacgcgaagaaccttaccaggtcttgacatcccctgacaacccaagagattgggcgttccttcgggggacagggtgacaggtggtgcatggttgtcgtcagctcgtgtcgtgagatgttgggttaagtcccgcaacgcgcgcaaccctcgcctctagttgccagcacgaaggtgggcactctagagggactgccggtgacaagtcggaggaaggtggggatgacgtcaaatcatcatgccccttatgacctgggctacacacgtgctacaatgggcggtacaaagggctgcgaacccgcgagggggagcgaatcccaaaaagccgctctcagttcggattgcaggctgcaactcgcctgcatgaagccggaatcgctagtaatcgcggatcagcatgccgcggtgaatacgttcccgggccttgtacacaccgcccgtcacaccacgagagctcgcaacaccc

SEQ ID NO: 121 - DNA, 1528 bp, Artificial Sequence
  Description of Artificial Sequence Figure 8 consensus sequence
agagtttgatcctggctcaggacgaacgctggcggcgtgcctaatacatgcaagtcgagcggaccncttcggnggtcagcggcggacgggtgagtaacacgtgggcaatctgccnnncagaccggaataacnccnggaaacgggtgctaatgccggatannncncgagnaggcatctncttgnggngaaaggtgcaantgnatcgctganngaggagcccgcggcgcattagctagttggtgnggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcgggaagagcggcanggngngtggaaagcnccntgngagacggtaccgagngaggaagccccggctaactacgtgccagcagccgcggtaatacgtagggggcaagcgttgtccggaatcactgggcgtaaagcgtgcgtaggcggttgngtaagtctgnngtgaaagtccanggctcaaccntggganngcnttggaaactgcntgacttgagtgctggagaggcaaggggaattccacgtgtagcggtgnaatgcgtaganatgtggaggaataccagtggcgaaggcgccttgctggacagtgactgacgctgaggcacgaaagcgtggggagcaaacaggattagataccctggtagtccacgccgtaaacgatgagtgctaggtgttggggggtcacacccncagtgccgaaggaaacccaataagcactccgcctggggagtacggtcgcaagactgaaactcaaaggaattgacgggggcccgcacaagcagtggagcatgtggtttaattcgaagcaacgcgaagaaccttaccagggcttgacatcccnctgacanccnnagagatgcgnnntcccttcggggcagnggagacaggtggtgcatggttgtcgtcagctcgtgtcgtgagatgttgggttaagtcccgcaacgagcgcaacccttganctgtgttaccagcacgtnnaggtggggactcacaggtgactgccggcgtaagtcggaggaaggcggggatgacgtcaaatcatcatgccccttatgtcctgggctacacacgtgctacaatgggcggtacaangggangcgaanccgcgaggnggagcnaancccanaaagccgntcgtagttcggattgcaggctgcaactcgcctgcatgaagccggaattgctagtaatcgcggatcagcatgccgcggtgaatncgttcccgggccttgtacacaccgcccgtcacaccacgagagtcggcaacacccgaagtcggtgnggtaacccntnnngggngccagccgccgaaggtggggnngatgattggggtgaagtcgtaacaaggtagccgtnnnnnnnnnnnnnnnnnnnnnnnnnnnn

SEQ ID NO: 122 - DNA, 1576 bp, Artificial Sequence
  Description of Artificial Sequence Figure 8 consensus sequence
nnnnnnnnnnnnnnnnnnnnngangaacgctggcggcgtgcctaanacatgcaagtcgnnnnnnnnnnnnnnnngnnnnnnnnnnnnnnnnnnnnnnnnnnnnnnagcggcggacgggtgagnaacncgtgggnaancnncnnnnnnnnnnggnanaacnnnnggaaacnngngctaannccnnatannnnnnnnnnnnngcntnnnnnnnnnnngnaagnnnnnnnnngnnnnncnnnnnnngangngcccgcgncnnattagctngttggnnnggtaacggnnnaccaaggcnnngatnngtagccgncctgagagggtgnncggccacactggnactgagacacggnccagactcctacgggaggcagcagtngggaatnttncncaatggnngnaannctgangnagcnacgccgcgtnagcgaagaaggccttcgggtngtaaagctnngtnnnnngggnnnnagnnnnnnnnnnnnnnnnnnnnnnnnnnnnngacggtaccnnnngagnaagccccggcnaantacgtgccagcagccgcggtaanacgtagggggcnagcgttntccggaatnantgggncgtaaagngngcgnangcggnnnnnnnngtcngnnntgaaagnnnnnggctcnaccntnnnnnnncnttngaaactgnnnnnncttgagtgcnggagaggnnagnnnaattccnngtgtnancggtgnaantgcgnnananatnnggaggaanaccagtggcnaangcgnctnnnctggncngnnnnnnnnnctgannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngcngnagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncncgaanancnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngggnannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncaannggnnnnnnnnnnnnnnnnnnnnnnnnnnnnnnnnnnnnnnnnnnnnnnnnnnnttnnnnnnnnnnnnnnnnnnnnnnnnnnnnnnnnnnnnnnnnnnnnnnnnnnnnnnnnnnnnnnnnnnnnnnntncccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnn

SEQ ID NO: 123 - DNA, 1526 bp, Artificial Sequence
  Description of Artificial Sequence Figure 8 majority sequence
agagtttgatcctggctcaggacgaacgctggcggcgtgcctaatacatgcaagtcgagcggacccggcggaggtcagcggcttacgggtgagtaacacgtgggcaatctgcctttcagaccggaataacgcccggaaacgggtgctaatgccggataacccgcgaggaggcatcttcttgcggggaaaggtgcaattgcatcgctgagggaggagcccgcggcgcattagctagttggtggggtaacggctcaccaaggcgacgatgcgtagccgacctgagagggtgaccggccacactgggactgagacacggcccagactcctacgggaggcagcagtagggaatcttccgcaatgggcgcaagcctgacggagcaacgccgcgtgagcgaagaaggccttcgggttgtaaagctctgttgctcgggaagagcggcaaggggagtggaaagccccttgcgagacggtaccgagtgaggaagccccggctaactacgtgccagcagccgcggtaatacgtagggggcaagcgttgtccggaatcactgggcgtaaagcgtgcgtaggcggttgcgtaagtctggggtgaaagtccagggctcaaccgtgggaatgctttggaaactgcgtgacttgagtgctggagaggcaaggggaattccacgtgtagcggtgaaatgcgtagatatgtggaggaataccagtggcgaaggcgccttgctggacagtgactgacgctgaggcacgaaagcgtggggagcaaacaggattagataccctggtagtccacgccgtaaacgatgagtgctaggtgttggggggtcacaccctcagtgccgaaggaaacccaataagcactccgcctggggagtacggtcgcaagactgaaactcaaaggaattgacggggcccgcacaagcagtggagcatgtggtttaattcgaagcaacgcgaagaaccttaccagggcttgacatccctctgacagccgcagagatgcggtttcccttcggggcaggggagacaggtggtgcatggttgtcgtcagctcgtgtcgtgagatgttgggttaagtcccgcaacgagcgcaacccttgacctgtgttaccagcacgttaaggtggggactcacaggtgactgccggcgtaagtcggaggaaggcggggatgacgtcaaatcatcatgccccttatgtcctgggctacacacgtgctacaatgggcggtacaacgggaagcgaagccgcgaggtggagcgaaccccaaaaagccgctcgtagttcggattgcaggctgcaactcgcctgcatgaagccggaattgctagtaatcgcggatcagcatgccgcggtgaatccgttcccgggccttgtacacaccgcccgtcacaccacgagagtcggcaacacccgaagtcggtgaggtaacccgtgtagggagccagccgccgaaggtggggtcgatgattggggtgaagtcgtaacaaggtagccgtnnnnnnnnnnnnnnnnnnnnnnnnnnnn

SEQ ID NO: 124 - DNA, 134 bp, Alicyclobacillus acidocaldarius
cgtagttcggattgcaggctgcaactcgcctgcatgaagccggaattgctagtaatcgcggatcagcatgccgcggtgaatacgttcccgggccttgtacacaccgcccgtcacaccacgagagtcggcaacac

SEQ ID NO: 125 - DNA, 134 bp, Alicyclobacillus acidocaldarius; identical to SEQ ID NO: 124

SEQ ID NO: 126 - DNA, 134 bp, Alicyclobacillus cycloheptanicus
cgtagttcggattgcaggctgcaactcgcctgcatgaagccggaattgctagtaatcgcggatcagcatgccgcggtgaatccgttcccgggccttgtacacaccgcccgtcacaccacgagagtcggcaacac

SEQ ID NO: 127 - DNA, 134 bp, Alicyclobacillus cycloheptanicus; identical to SEQ ID NO: 126

SEQ ID NO: 128 - DNA, 134 bp, Alicyclobacillus acidoterrestris; identical to SEQ ID NO: 126

SEQ ID NO: 129 - DNA, 134 bp, Alicyclobacillus acidoterrestris; identical to SEQ ID NO: 126

SEQ ID NO: 130 - DNA, 134 bp, Clostridium elmenteitii
cccagttcggattgagggctgcaactcgcccccatgaagttggagttgctagtaatcgcgaatcagcatgtcgcggtgaatgcgttcccgggtcttgtacacaccgcccgtcacaccacggaagtcggaagcac

SEQ ID NO: 131 - DNA, 134 bp, Geobacillus subterraneus
ctcagttcggattgcaggctgcaactcgcctgcatgaagccggaatcgctagtaatcgcggatcagcatgccgcggtgaatacgttcccgggccttgtacacaccgcccgtcacaccacgagagcttgcaacac

SEQ ID NO: 132 - DNA, 134 bp, Sulfobacillus disulfidooxidans
ctcagttcggattgcaggctgcaactcgcctgcatgaagccggaatcgctagtaatcgcggatcagcatgccgcggtgaatccgttcccgggccttgtacacaccgcccgtcacaccacgagagtcgacaacac

SEQ ID NO: 133 - DNA, 134 bp, Bacillus thermoleovorans
ctcagttcggattgcaggctgcaactcgcctgcatgaagccggaatcgctagtaatcgcggatcagcatgccgcggtgaatacgttcccgggccttgtacacaccgcccgtcacaccacgagagctcgcaacac

SEQ ID NO: 134 - DNA, 1500 bp, Alicyclobacillus acidocaldarius; identical to SEQ ID NO: 74

SEQ ID NO: 135 - DNA, 1519 bp, Alicyclobacillus acidoterrestris; identical to SEQ ID NO: 75
  modified_base (549)..(549): a, c, g, or t

SEQ ID NO: 136 - DNA, 1497 bp, Alicyclobacillus cycloheptanicus; identical to SEQ ID NO: 76
  modified_base (967): a, t, c or g

SEQ ID NO: 137 - DNA, 1093 bp, Zygosaccharomyces sp.
  modified_base (14)..(14): a, c, g, or t
attgggccctctanagcatgctcgacggccgccagtgtgatggatatctgcagaattcggctttgcatggccgttcttagttggtggagtgatttgtctgcttaattgcgataacgaacgagaccttaacctactaaatagtggtgctagcatttgctggtttttccacnttcttagagggactatcggtttcaagccgatggaagtttgaggcaataacaggtctgtgatgcccttagacgttctgggccgcacgcgcgctacactgacggagccagcgagtctaaccttggccgagaggtctgggtaatcttgtgaaactccgtcgtgctggggatagagcattgtaattattgctcttcaacgaggaattcctagtaagcgcaagtcatcaacttgcgttgattacgtccctgccctttgtacacacaagccgaattccagcacactggcggccgttactagtggatccgagctcggtaccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcanangccagganccgtaaaaggccgcgtgctggcgttttncntangctcgccccctgacagcatncaaaatcgacgctcagtcnnangtggcgaacccgnngganataagatacnngcgttncccctgnanctccncntggctntcngntcnancngncgntanggaanctgncnccttcnccttnggaacnggnncttnnnnnnnancngnngnnnnnnnnnnggnnn

SEQ ID NO: 138 - DNA, 1112 bp, Penicillium digitatum
  modified_base (3)..(3): a, c, g, or t
gangncnncccnnantnnatcctnagcngagtngnnaagcgcncgttnccganggagaagnggacaggtntccgtancgcaggtnngancaggagagcgcacgagggagctncagggggaaacgcctgggatcttnatagtccngtcgggttcnccacntctgacttgagcgtcgattttgtgatgctcgtcagggggcggagcntatggaaaacgccagcaacgcggccttttacggttcctggcntttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcggctttgcatggccgttcttagttggtggagtgatttgtctgcttaattgcgataacgaacgagacctcggcccttaaatagcccggtccgcatttgcgggccgctggcttcttaaggggactatcggctcaagccgatggaagtgcgcggcaataacaggtctgtgatgcccttagatgttctgggccgcacgcgcgctacactgacagggccagcgagtacatcaccttaaccgagaggtttgggtaatcttgttaaaccctgtcgtgctggggatagagcattgcaattattgctcttcaacgaggaatgcctagtaggcacgagtcatcagctcgtgccgattacgtccctgccctttgtacacacaagccgaattctgcagatatccatcacactggcggccgtcgagcatgctntagagggcccaat

SEQ ID NO: 139 - DNA, 1094 bp, Byssochlamys fulva
  modified_base (1)..(131): a, c, g, or t
nnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncnnnnggnnnnncncntnnnnnnngnnnnnnnnnnnnnnnntntcnngggnnngngcnnnngnaaannnccngcannnngccnttnnnngntnnnggccntnngnngncnnnngntcacangttnntcnngcgntntcccnngnttnngnggataacngtattnccgcctnngagtgagntgataccgctcgcngcagccgaacgaccgagcgcagcgagtcagtgagcgaggaagcggaagagcgcncaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttggtaccgagctcggatccactagtaacggccgccagtgtgctggaattcggctttgcatggccgttcttagttggtggagtgatttgtctgcttaattgcgataacgaacgagacctcggctcttaaatagcccggtccgcgtttgcgggccgctggcttcttagggggactatcggctcaagccgatggaagtgcgcggcaataacaggtctgtaatgcccttagatgttctgggccgcacgcgcgctacactgacagggccagcgggtacatcaccttggccgagaggtctgggtaatcttgttaaaccctgtcgtgctggggatagagcattgcaattattgctcttcaacgaggaatgcctagtaggcacgagtcatcagctcgtgccgattacgtccctgccctttgtacacacaagccgaattctgcagatatccatcacactggcggccgtcgagcatgctntagagggcccaat

SEQ ID NO: 140 - DNA, 878 bp, Artificial Sequence
  Description of Artificial Sequence Figure 5 majority sequence
gggggttggatgttccaggcttgtatttctccagtgtgggatactggcttggccgtgttggcgctgcgttctgctgggtttccggccgatcatgccgggttggttaaggcgggtgagtggttgttgggtcggcagattctcgtggctggcgactgggaggttcgtcgccggaaggtgaaaccgggcggtttggcgtttgagttcgactgcgtgtactacccggacgtggacgatacggcggtggtcgtcttggcgctcaatggccttcgattgccggatgaggggcggcgtcgtgacgccttgacgcgtggcttccgttggtttgtcgggatgcagagttcgaacgggggctggggcgcatacgatgtggacaacacgcgtgatttgccgaatcggattccgttttgcgacttcggcgaagtgattgatccgccgtcggaagacgtcaccgcccacgtgttggagtgtttcggcagctttgggtacgacgaggcctggaaggtgattcggcgggcggtggagtatctcaagggggagcagcggccggatgggtgctggtttggtcgctggggcgtcaactacgtgtatggcatgggcgcggtggtttcggggctgaaggcggtcggtgtcgatatgcgtgagccgtgggttcaaaagtcgctcgactgggtcgtggagcatcagaatgcggatggcggctggggtgaagactgccgntcntacgaggatccgnnnctcgcgggtcagggcgcgagnacaccgtcgcagacngcctgggcgttgatggcgctcatcgcgggcggcngtgtcgagtcagangccgcacnncgcggggtccnntacctnnnnganacgcagcgcgcngatggtggctgnnn

(19) United States
(12) Patent Application Publication
(10) Pub. No.: US 2009/0117557 A1
(43) Pub. Date: May 7, 2009

(54) Rapid Detection of Microorganisms

(75) Inventors: Hua Wang, Columbus, OH (US); Hongliang Luo, Columbus, OH (US); Chris Connor, Columbus, OH (US); Steven Schwartz, Powell, OH (US); Ahmed Yousef, Columbus, OH (US); Kai Wan, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY RESEARCH FOUNDATION, Columbus, OH (US)

(21) Appl. No.: 11/939,492

(22) Filed: Nov. 13, 2007

Related U.S. Application Data
(63) Continuation of application No. 10/727,261, filed on Dec. 2, 2003, now abandoned.
(60) Provisional application No. 60/513,246, filed on Oct. 22, 2003.
(60) Provisional application No. 60/500,736, filed on Sep. 5, 2003.
(60) Provisional application No. 60/430,202, filed on Dec. 2, 2002.

(51) Int. Cl. C12Q 1/68

(52) U.S. Cl. 435/6

(57) ABSTRACT

Tools and methods for detecting the presence bacteria, yeast and mold in a sample obtained from a food sample are provided. The methods employ a polymerase chain reaction and primer and probe sets that are based on the 16S rRNA and squalene-hopene cyclase genes of Alicyclobacillus and Geobacillus and the 18S rDNA gene of mold and yeast. The present invention also relates to primer and probe sets. Each primer and probe set comprises a forward primer and a reverse primer, both of which are from 15 to 35 nucleotides in length and a probe.

### CROSS REFERENCE TO RELATED APPLICATIONS

This application claims priority to U.S. Provisional Applications No. 60/______, filed Oct. 22, 2003, No. 60/500,736, filed Sep. 5, 2003, and No. 60/430,202, filed Dec. 2, 2002, each of which is incorporated herein by reference in their entirety.

### TECHNICAL FIELD OF THE INVENTION

The present invention provides methods and tools for rapidly detecting microorganisms such as molds and fungi, and acid and thermophilic Alicyclobacillus spp and Geobacillus spp. in test samples, particularly food samples.

### BACKGROUND

Spoilage of products, particularly food and beverage products, due to contamination with bacteria, yeasts and molds, results in significant financial loss to the food industry. Yeasts and molds are commonly associated with raw materials of foods and are often found in the processing environment. Due to the structural features of both the vegetative cells and spores of fungi, these food contaminants have a good chance of surviving current processing conditions. Yeasts and molds can grow within a wide range of environmental conditions, and therefore the presence in food of even minor amounts of yeast and mold contaminants can cause spoilage during storage.

Like fungi, many bacteria are resistant to processing conditions, and some are resistant even to high acid conditions in food and beverage products. Alicyclobacilli are Gram-positive, spore-forming, aerobic rods classified as thermoacidophiles capable of growing at high temperatures and low pH (1, 2, 3). These bacteria, formerly of the Bacillus genus, were assigned into the new genus Alicyclobacillus in 1992 (1). Sequence analysis of the 16s rRNA genes proved that three previously classified Bacillus thermoacidophiles (B. acidocaldarius, B. acidoterrestris, and B. cycloheptanicus) belong in a group that differs from other closely related Bacilli. Additionally, a key phenotypic variation was found in the membrane composition of these three species. The primary fatty acid component in the membrane was determined to be ω-alicyclic fatty acids, a type of lipid not found in other Bacillus species at the time. This evidence initiated the establishment of the Alicyclobacillus genus of obligate acidothermophiles, containing A. acidocaldarius, A. acidoterrestris, and A. cycloheptanicus, within the Bacillus branch (1). More recently, A. hesperidum and Alicyclobacillus genomic species 1 and 2 (24, 25), A. acidiphilus (22), A. herbarius (23), A. sendaiensis (26), and A. pomorum (27) have been added as new species within the genus Alicyclobacillus.

Alicyclobacilli have been an increasingly frequent spoilage problem in the beverage industry, particularly acidic juices, during the last two decades. In 1982, a Bacillus sporeformer (to be later classified as B. acidoterrestris and then subsequently A acidoterrestris) capable of growing at pH as low as 2.5 was isolated from apple juice (4, 5, 6). In 1994, Splittstoesser et al. discovered the presence of A. acidoterrestris in apple juice, further shown by Yamazaki et al. in 1996 (7, 8). Spore germination and growth in orange juice (3) and grapefruit juice (6) was even observed. White grape juice, tomato juice, cranapple juice, and pear juice have also been afflicted with Alicyclobacillus spoilage (11).

While Alicyclobacilli are non-pathogenic, they are a spoilage agent that can drastically affect the quality of acidic fruit juices. Pettipher et al. (1997) reported that guiacol, one of the chemicals responsible for the off-odor and smoky taints characteristic in Alicyclobacillus-spoiled juices, can be detected by taste before any visible contamination is seen (3). Therefore, a consumer would generally not be able to identify Alicyclobacillus-spoiled juice until it is ingested. In addition to guiacol, 2,6-dibromophenol (2,6-DBP) and 2,6-dichlorophenol (2,6-DCP) were found to contribute to disinfectant taints at detectable levels after as little as one day at 44° C. in containers with large headspaces. More realistically, commercially stored shelf stable juices with generally low headspace volume develop these taints within the first month of storage, particularly in warmer climates (10). The presence of these chemicals in Alicyclobacillus-spoiled juices significantly reduces the quality of the product, subsequently lowering the consumer image of the brand.

Alicyclobacilli are very heat resistant, growing from pH 2.5-5.5 and 25° C.-60° C. (6). Beyond growth, cells and spores can survive normal pasteurization procedures, at temperatures up to 97° C. (3,6). Fruit juices that are fresh squeezed, pasteurized, or hot-filled are most easily affected by Alicyclobacillus spoilage, since ultra high temperature treatment is normally sufficient for killing all microorganisms (3). Since Alicyclobacilli can survive temperatures that exceed industry standard pasteurization specifications, contamination occurring before or during the processing steps can lead to spoilage in the final product that reaches the consumer. Since significant increases in pasteurization temperatures or times ultimately affect product quality and flavor, companies aren't likely to change current procedures.

Early detection, i.e., before products reach the consumer, of the presence of even small amounts of these microbial contaminants in food and beverages is highly desirable in the food industry. Classic culture methods are generally accurate for detecting the presence of microorganisms, but can take up to a week for the results. Previdi et al. (1997) reported a method for detecting A. acidocaldarius in juice products. This method required juices or concentrates to be heat treated and then incubated at 37° C. for 7 days, followed by plating on pH 4.0 malt extract agar (13). Pinhatti et al. (1997) tested frozen orange juice concentrate by heat shocking the samples at 80° C., enriching at 50° C. for 24 and 48 h, and finally pour plating in BAM and incubating at 50° C. for 24 h (12). Both of these methods of detection provided accurate results, but took from 3-7 days to complete. As with bacteria, it can often take one to two weeks just to grow yeast and mold cells on culture media. In addition, there are so many varieties of molds and yeasts with diverse growth requirements that it is very difficult to find an optimal medium to capture all potential yeast and mold contaminants at the same time. For food industry applications, it is desirable to have a rapid detection system that does not require time consuming culture techniques to detect the presence of microbial contamination of food samples. Accordingly, it is desirable to have a more rapid detection method that can provide results within a few hours, with the same level reliability of culture methods. It is also desirable to have kits that can differentiate between specific types of microbes and which comprise microbe-specific reagents that are useful for conducting rapid sample testing.

### SUMMARY OF THE INVENTION

The present invention provides methods and kits for detecting the presence of Alicyclobacillus spp. and a closely related thermophilic bacterium, Geobacillus, in samples, particularly food samples. In one embodiment the method comprises, collecting bacterial cells in the sample, extracting DNA from the cells, and assaying for the presence of these bacterium species using a PCR technique, preferably real-time PCR, and three signature oligonucleotides (2 primers and a probe) directed to a particular sequence in a target gene encoding either the 16S rRNA or squalene-hopene cyclase (shc). (See the conserved sequences extending from nucleotide position 334 through nucleotide position 485, and from nucleotide position 752 through nucleotide position 813 of the shc gene sequence of Alicyclobacillus shown in FIG. 5. Also see the conserved sequences extending from nucleotide position 1327 through nucleotide position 1460 of the 16S rRNA gene sequence of Alicyclobacillus shown in FIG. 1.) The presence of multiple Alicyclobacillus spp. and a closely related thermophilic bacterium Geobacillus can be achieved within 3-5 hours using the described sample preparation procedures, and proper combination of the three oligonucleotides as primer-and-probe set in the real-time PCR reaction.

The kits of the present invention comprise at least one forward primer and one reverse primer, with or without a probe for amplifying a sequence of at least 50 consecutive nucleotides within a conserved region of the three Alicyclobacillus spp. shown in FIG. 1 (sequences shown in alignment). FIGS. 2, 3 and 4, respectively, show the full coding sequences for the 16S rRNA genes from the Alicyclobacillus strains deposited with the ATCC as 43030, 49025, and 49029. In certain embodiments, the oligonucleotides comprise the entire or a majority of the following sequences or their reverse complement sequences, as a set or as combination crossing multiple sets, e.g. in certain cases the forward primer of one set can be combined with a reverse primer that is based on the forward primer of another set. Thus the following embodiments can be used in various primer, probe, or primer-probe combinations. Depending on the primers that are combined, the lower oligo may be used as a probe. The sequence of the lower oligo corresponds to the coding sequence of the target region of the gene, and is complementary to the reverse primer in each set. The reverse primers are shown as the reverse complement of the targeted region of the gene. The forward primers correspond to the coding sequence of the target region of the gene.

TABLE I
Signature Oligonucleotides Directed Toward 16S rRNA gene

| | | |
|---|---|---|
| Length | Tm(° C.) | GC % |

Set 1:

| | | | | |
|---|---|---|---|---|
| Forward primer: 5′GAGCCCGCGGCGCATTAGC3′ | 19 | 68.9 | 73.7 | (SEQ ID NO 1) |
| Probe: 5′GCGACGATGCGTAGCC(G)3′ | 16 | 61.8 | 68.8 | (SEQ ID NO 2) |
| Lower Oligo: 5′CGCAATGGGCGCAAGC3′ | 16 | 61.8 | 68.8 | (SEQ ID NO 3) |
| Reverse primer: 5′GCTTGCGCCCATTGCG3′ | 16 | 61.8 | 61.8 | (SEQ ID NO 4) |
| Set 2: | | | | |
| Forward primer: 5′GAGCAACGCCGCGTGAGCG3′ | 19 | 68.8 | 73.7 | (SEQ ID NO 5) |
| Probe: 5′CTTCGGGTTGTAAAGC3′ | 16 | 54.2 | 50 | (SEQ ID NO 6) |
| Lower Oligo: 5′CGGCTAACTACGTGC3′ | 15 | 56.2 | 60 | (SEQ ID NO 7) |
| Reverse primer: 5′GCACGTAGTTAGCCG5′ | 15 | 56.2 | 60 | (SEQ ID NO 8) |
| Set 3: | | | | |
| Forward Primer: 5′AGTGCTGGAGAGGCAAGG3′ | 18 | 62.2 | 61.1 | (SEQ ID NO 9) |
| Probe: 5′CTGGACAGTGACTGACG3′ | 17 | 59.6 | 58.8 | (SEQ ID NO 10) |
| Lower Oligo 5′GCACGAAAGCGTGGGGAGCA | 20 | 66.6 | 65 | (SEQ ID NO 11) |
| Reverse Primer: 5′TGCTCCCCACGCTTTCGTGC5′ | 20 | 66.6 | 65 | (SEQ ID NO 12) |
| Set 4: | | | | |
| Forward Primer: 5′GGAGTACGGTCGCAAGACTG3′ | 20 | 64.5 | 60 | (SEQ ID NO 13) |
| Probe: 5′CGCACAAGCAGTGGAGC3′ | 17 | 62.0 | 64.7 | (SEQ ID NO 14) |
| Lower Oligo: 5′CAGGGCTTGACATC3′ | 14 | 52.6 | 57.1 | (SEQ ID NO 15) |
| Reverse Primer: 5′GATGTCAAGCCCTG3′ | 14 | 52.6 | 57.1 | (SEQ ID NO 16) |
| Set 5: | | | | |
| Forward primer: 5′GGCGTAAGTCGGAGGAAGG3′ | 19 | 64.5 | 63.2 | (SEQ ID NO 17) |
| Probe: 5′ATGTCCTGGGCTACACACG3′ | 19 | 62.3 | 57.9 | (SEQ ID NO 18) |
| Reverse primer: 5′GCCTGCAATCCGAACTACC5′ | 19 | 62.3 | 57.9 | (SEQ ID NO 19) |
| Set CC16S: | | | | |
| Forward primer: 5′CGTAGTTCGGATTGCAGGC3′ | 19 | 65.6 | 57.9 | (SEQ ID NO 20) |
| Probe: 5′CGGAATTGCTAGTAATCGCG3′ | 20 | 57.9 | 47.4 | (SEQ ID NO 21) |
| Lower Oligo: 5′CACGAGAGTCGGCAACAC3′ | 18 | 63.3 | 61.1 | (SEQ ID NO 22) |
| Reverse primer: 5′GTGTTGCCGACTCTCGTG3′ | 18 | 62.2 | 61.1 | (SEQ ID NO 23) |
| Set 6: | | | | |
| primer: 5′GATGATTGGGGTGAAG3′ | 16 | 54.2 | 50 | (SEQ ID NO 24) |

TABLE II

Signature Oligonucleotides Directed Toward squalene-hopene cyclase
(shc) gene

These three oligonucleotides were further used as PCR primer pair and
DNA probe in real-time
PCR detection of Alicyclobacillus spp.

| | |
|---|---|
| Forward Primer: 5′ATGCAGAGYTCGAACG 3′ | (SEQ ID NO 25) |
| Probe: 5′6-FAM d [TCG(A)GAA(G)GACGTCACCGC] BHQ-1 3′ | (SEQ ID NO 26) |
| Reverse Primer: 5′AAGCTGCCGAARCACTC 3′(Y = C + T; R = A +G | (SEQ ID NO 27) |

TABLE III

The Sequence, GC % and Tm of Primer and probe set
candidate 1 for Shc Gene:

| | | | | |
|---|---|---|---|---|
| Name | Sequence | Length | Tm | GC % |

| | | | | |
|---|---|---|---|---|
| Forward primer | TACTGGTGGGGGCCGCT (SEQ ID NO 28) | 17 | 64.84 | 70.59 |
| | TACTGGTGGGCGCCGCT (SEQ ID NO 29) | 17 | 64.84 | 70.59 |
| Probe | ATGGAAGCGGAGTACGTCC (SEQ ID NO 30) | 19 | 62.64 | 57.9 |
| | ATGGAAGCGGAGTACGTCCT (SEQ ID NO 31) | 20 | 62.45 | 55 |
| | ATGGAAGCGGAATATGTGC (SEQ ID NO 32) | 19 | 58.32 | 47.37 |
| | ATGGAAGCGGAATATGTGCT (SEQ ID NO 33) | 20 | 58.35 | 45 |
| Reverse Primer | CGCGAGGACGGCAC (SEQ ID NO 34) | 14 | 62.11 | 78.57 |
| | CGCGAGGACGGCACGTGG (SEQ ID NO 35) | 18 | 69.79 | 77.78 |
| | CGCGAAGACGGCAC (SEQ ID NO 36) | 14 | 59.16 | 71.43 |
| | CGCGAAGACGGCACCTGG (SEQ ID NO 37) | 18 | 67.51 | 72.22 |

TABLE IV

The Sequence, GC % and Tm of Primer and probe set candidate
2 for Shc Gene:

| | | | | |
|---|---|---|---|---|
| Name | Sequence | Length | Tm | GC % |

| | | | | |
|---|---|---|---|---|
| Forward | CAAAAGGCGCTCGACTG (SEQ ID NO 38) | 17 | 60.02 | 58.82 |
| primer | CAAAAGGCGCTCGACTGG (SEQ ID NO 39) | 18 | 62.96 | 61.11 |
| | CAAAAGGCGCTCGACTGGGTCG (SEQ ID NO 40) | 22 | 68.99 | 63.64 |
| | CAAAAGTCGCTCGACTG (SEQ ID NO 41) | 17 | 57.61 | 52.94 |
| | CAAAAGTCGCTCGACTGG (SEQ ID NO 42) | 18 | 60.68 | 55.56 |
| | CAAAAGTCGCTCGACTGGCTCG (SEQ ID NO 43) | 22 | 67.13 | 59.09 |
| Probe | GGACGGCGGCTGGGGCGA (SEQ ID NO 44) | 18 | 72.07 | 83.33 |
| | GGACGGCGGCTGGGGCGAGGA (SEQ ID NO 45) | 21 | 75.09 | 80.95 |
| | GGACGGCGGCTGGGGCGAGGACTGCCG | 27 | 80.31 | 81.48 |
| | (SEQ ID NO 46) | | | |
| | GGATGGCGGTTGGGGTGA (SEQ ID NO 47) | 18 | 65.23 | 66.67 |
| | GGATGGCGGTTGGGGTGAAGA (SEQ ID NO 48) | 21 | 67.28 | 61.91 |
| | GGATGGCGGTTGGGGTGAAGATTGCCG | 27 | 72.72 | 62.96 |
| | (SEQ ID NO 49) | | | |
| Reverse | TGATGGCGCTCATCGC (SEQ ID NO 50) | 16 | 59.53 | 62.5 |
| Primer^{a} | | 23 | 74.2 | 73.91 |
| 1 | TGATGGCGCTCATCGCGGGCGGC (SEQ ID NO 51) | | | |
| 2 | ACCCCGTCGCAGACGGCCTGGGCGC | 25 | 77.7 | 80 |
| | (SEQ ID NO 52) | | | |
| 3 | ACACCGTCGCAGACCGCCTGGGCGT | 25 | 74.42 | 72 |
| | (SEQ ID NO 53) | | | |

The present invention also provides methods and kits for detecting the presence of yeast and mold contaminants in samples, particularly in food samples. In one aspect, the method comprises collecting particulate matter, preferably cells and cellular fragments, in the sample, extracting DNA from the particulate matter, and assaying for the presence of yeast DNA in the extracted DNA using a PCR technique using primers that amplify a select conserved region in 18s rDNA of representative yeast species, including Zygosaccharomyces bailii (Lindner) Guilliermond strain ATCC 36947 and the other yeast species shown FIG. 7. (See conserved sequence extending from nucleotide 81 through nucleotide 225 of the sequence of Z. bali.)

Preferably, the method uses real-time PCR, and three signature oligonucleotides (2 primers and a probe) directed to a particular sequence in the gene encoding the yeast 18S rDNA.

In another aspect, the kit of the present invention comprise at least one forward primer and one reverse primer, with or without a probe for amplifying a sequence of at least 50 consecutive nucleotides within the select region of yeast 18s rDNA. In one embodiment, the kit comprises primers and a probe having the following sequences:

(SEQ ID NO 54)

Yupreal: 5′GTGGTGCTAGCATTTGCTG 3′

(SEQ ID NO 55)

Ylowreal: 5′GTTAGACTCGCTGGCTCC 3′

(SEQ ID NO 56)

Yprobe: 5′TTTCAAGCCGATGGAAGTTTGA(C/G)3′

Another probe that may be used in the present method has the following sequence

5′CGGTTTCAAGCCGATGGAAGT 3′. (SEQ ID NO 57)

Yet another set of primers and probe for yeast detection:

Oligo name

| | | | |
|---|---|---|---|
| Len | Pur | Scale | Sequence (5′-3′) |

18srRNA-newup-112503-1

| | | | | |
|---|---|---|---|---|
| 30 | DST | 0.05 | CCTACTAAATAGGGTGCTAGCATTTGCTGG | (SEQ ID NO 58) |

18srRNA-newup-112503-2

| | | | | |
|---|---|---|---|---|
| 26 | DST | 0.05 | CTAAATAGGGTGCTAGCATTTGCTGG | (SEQ ID NO 59) |

18srRNA-probe2

| | | |
|---|---|---|
| 25 | CGGTTTCAAGCCGATGGAAGTTTGA | (SEQ ID NO 60) |

In another aspect the present method comprises collecting particulate matter, preferably cells and cellular fragments, in the sample, extracting DNA from the particulate matter, and assaying for the presence of mold DNA in the extracted DNA using a PCR technique using primers that amplify a select conserved region in 18s rDNA of the following representative molds: Byssochlamys fulva Olliver et Smith, teleomorph ATCC 24474 and Penicillium digitatum Saccardo, anamorph ATCC10030, as shown in the attached alignment. (See the conserved sequence extending from nucleotide 114 through nucleotide 239 of the 18s rDNA sequence of P. digitatum shown in FIG. 7.) Preferably, the method uses real-time PCR, and three signature oligonucleotides (2 primers and a probe) directed to a particular sequence in the gene encoding the mold 18s rDNA.

In another aspect, the present the kits of the present invention comprise at least one forward primer and one reverse primer, with or without a probe for amplifying a sequence of at least 50 consecutive nucleotides within the select region of mold 18s rDNA. In one embodiment, the kit comprises primers and a probe having the following sequences:

Mupreal: 5′CCGCTGGCTTCTTAGGG 3′ (SEQ ID NO 61)
Mlowreal: 5′AGGGCCAGCGAGTACATCA 3′ (SEQ ID NO 62)
Mprobe: 5′CTCAAGCCGATGGAAGTGCG 3′ (SEQ ID NO 63)

The invention further provides a method for detecting through real-time PCR using at least one of the nucleic acid primer pairs, and at least one probe, the presence of acidophilic bacterium in a test sample, especially in a food sample. In one embodiment, the acidophilic bacterium detection method includes use of one forward primer directed to the 16S rRNA gene, wherein the primer is selected from the forward primers/listed in Table I, one reverse primer directed to the 16S rRNA gene wherein the primer is selected from the reverse primers listed in Table I, and one probe directed to a sequence that is located between the sequences to which the forward and reverse primers are directed, wherein the probe is selected from the group of probles listed in Table I.

In another embodiment, the acidophilic bacterium detection method includes use of one forward primer directed to the squalene-hopene cyclase gene, wherein the forward primer is selected from the group of forward primers listed in Tables II and III, one reverse primer directed to the squalene-hopene cyclase gene wherein the primer is selected from the group of reverse primers listed in Tables II and III, and one probe directed to a sequence that is located between the sequences to which the forward and reverse primers are directed, wherein the probe is selected from the group of probes listed in Tables II and III.

In yet another embodiment, the acidophilic bacterium detection method includes use of one forward primer directed to the squalene-hopene cyclase gene, wherein the forward primer is selected from the group of forward primers listed in Table IV, one reverse primer directed to the squalene-hopene cyclase gene wherein the primer is selected from the group of reverse primers listed in Table IV, and one probe directed to a sequence that is located between the sequences to which the forward and reverse primers are directed, wherein the probe is selected from the group of probes listed in Table IV.

In another embodiment, the yeast detection method includes use of one forward primer directed to the 18S rDNA gene, wherein the primer is selected from the group consisting of SEQ ID NO 54 and SEQ ID NO 58, one reverse primer directed to the 18S rDNA gene wherein the primer is selected from the group of consisting of SEQ ID NO 55 and SEQ ID NO 55, and one probe directed to a sequence that is located between the sequences to which the forward and reverse primers are directed, wherein the probe is selected from the group consisting of SEQ ID NO 56, SEQ ID NO 57 and SEQ ID NO 60.

In yet another embodiment, the mold detection method includes use of one forward primer directed to the 18S rDNA gene, wherein the primer corresponds to SEQ ID NO 61, one reverse primer directed to the 18S rDNA gene wherein the primer corresponds to SEQ ID NO 62, and one probe directed to a sequence that is located between the sequences to which the forward and reverse primers are directed, wherein the probe corresponds to SEQ ID NO 63.

### BRIEF DESCRIPTION OF THE DRAWINGS

FIG. 1 shows polynucleotide sequence alignment of 16S rRNA gene fragments from three representative strains of Alicyclobacillus, specifically, A. acidocaldarius ATCC43030, A. acidoterrestris ATCC49025, and A. cycloheptanicus ATCC49029

FIG. 2 shows the 16S rRNA gene coding Sequence for A. cycloheptanicus ATCC49029

FIG. 3 shows the 16S rRNA gene coding Sequence for A. acidoterrestris ATCC49025

FIG. 4 shows the 16S rRNA gene coding Sequence for A. acidocaldarius ATCC43030

FIG. 5: shows the Shc gene sequence alignments for A. cycloheptanicus ATCC49029 and A. acidoterrestris ATCC49025

FIG. 6: shows the Shc amino acid sequence alignments for A. cycloheptanicus ATCC49029 and A. acidoterrestris ATCC49025

FIG. 7 shows the alignment for the 18s rDNA gene coding Sequence for Zygosaccaromyces, Penecillium digitatum, and Byssochlamys fulva

FIG. 8 shows the 16S rRNA gene coding sequence alignments for several strains of for A. cycloheptanicus

FIG. 9. shows the results of Real-time PCR detection of A. acidocaldarius (black), A. cycloheptanicus (blue), and A. acidoterrestris (lt. green) using the CC16S specific probe and primer pair.

FIG. 10 shows the results of Real-time PCR sensitivity test of A. acidoterrestris using the CC16S primers and probe

FIG. 11 shows the results of Real-time PCR sensitivity test of A. acidoterrestris in orange juice.

FIG. 12 shows the 18s rDNA gene coding Sequence for Zygosaccaromyces

FIG. 13 shows the 18s rDNA gene coding Sequence for Penecillium digitatum

FIG. 14 shows the 18s rDNA gene coding Sequence for Byssochlamys fulva

FIG. 15 shows the results of a specificity test. • Zygosaccharomyces bailii (Lindner) Guilliermond ATCC 36947; ▪ industry sample yeast. ♦ Byssochlamys fulva Olliver et Smith ATCC 24474; ▾H₂O control with extraction. ▴ H₂O control without extration.

FIG. 16 shows the results of a specificity test with ▾ yeast, ♦mold and acciobacillus and ▴ H₂O

FIG. 17 shows the results of a specificity test with □ Z.b(yeast).; ▴ B.F.(mold); ♦ Accidobacillus; ▾ water; Apple; ▾ green grape; and ▪Red grape.

FIG. 18 shows the results of a specificity test with ▪ Z.b.; ▴ B.F.; ♦ Accidobacillus and ▾ water.

FIG. 19 shows the results of a specificity test with ▪Orange1; ▴Orange2; ♦Orange Juice Supernatant; Orange Juice pellet; Yeast; and ▾H₂O

FIG. 20 shows the results of a specificity test with !Byssochlamys fulva Olliver et Smith, telomorph ATCC 24474; Penicillium digitatum Saccardo, anamorph ATCC 10030; #Zygosaccharomyces bailii (Lindner) Guillermond, telomorph deposited as Saccharomyces bailii Lindner, telomorph ATCC 36947; % Industry Mold 42; &Indusrty Mold 41; “Industry Mold 3; “Water (extracted); % water (not extracted)

FIG. 21 shows specificity test results with Bussochlamys fulva Olliver et Smith, teleomorph ATCC24474; water and Zygosaccharomyces bailii (Lindner) Guilliermond, telomorph deposited as Saccharomyces bailii Lindner, telomorph ATCC 36947; Acidobacillus acidoterrestris 49025.

FIG. 22 shows the Alignment^{a} of 134 bp priming region flanked by CC16S-F (CGTAGTTCGGATTGCAGGC), CC16S-Probe (CGGAATTGCTAGTAATCGC), and CC16S-R (CACGAGAGTCGGCAACAC)^{b}.

FIG. 23 shows the results of Real-time PCR detection of A. acidocaldarius ATCC 43030 (a), A. cycloheptanicus ATCC 49029 (+), and A. acidoterrestris ATCC 49025 () using the CC16S primer and probe set.

FIG. 24 shows the results of Real-time PCR sensitivity test of A. acidoterrestris ATCC 49025 in saline solution using the CC16S primers and probe

FIG. 25 shows the results of Real-time PCR sensitivity test of A. acidoterrestris ATCC 49025 in orange juice, using the CC16S primers and probe.

FIG. 26 shows the results of Real-time PCR detection of food-borne microorganisms using the developed primer-and-probe set.

FIG. 27 shows the results f Real-time PCR sensitivity test

FIG. 28. Real-time PCR detection of A. acidocaldarius ATCC43030 cells in apple juice using shr-specific primer-and-probe set.

### DETAILED DESCRIPTION OF THE INVENTION

The methods and kits provided herein enable the rapid and reliable detection of contaminating microorganisms that are found in test samples of products, preferably consumer products, and most preferably food products. The methods are especially suited for the detection of Alicyclobacillus spp. including A. acidocaldarius, A. acidoterrestris, A. cycloheptanicus, A. hesperidum, A. acidiphilus, A. herbarius, A. sendaiensis, and A. pomorum and Geobacillus stearothemophilus, and a variety of yeasts and mold. Other reported methods use conventional PCR (using a pair of oligonucleotides as primers) to detect the presence of Alicyclobacillus spp. (Obara and Niwa, 1998) which usually is associated with the problem of high background with non-specific PCR products.

According to the methods described herein, a sample is obtained from a test material, for example a sample of a fruit juice or other food product. The sample is processed to extract any polynucleotides in the sample, particularly polynucleotides from target organisms that may be present in the material. After extraction and processing according to methods described herein or otherwise known in the art, the sample is treated with reagents that comprise a forward primer oligonucleotide, a reverse primer oligonucleotide, and a labeled oligonucleotide probe, wherein the reagents are targeted for specific regions within the genome of target organisms. The sample is then processed according to PCR amplification methods. The PCR product is first amplified using the primers. Binding of the labeled probe to a target sequence within the PCR product that corresponds with a target region in the genomic DNA of the contaminating bacteria or mold signals the presence of contaminating microorganisms.

Therefore the combination of the three unique sequences and the real-time PCR technology ensured specific and sensitive detection of the presence of the target bacteria. This real-time PCR approach also offers other features such as a) accuracy: more than one probe will be included in the detection system with less possible error; b) flexibility: up to four PCR products can be simultaneously detected so potentially incorporating probes for other spoilage microorganisms into the detection system is expected.

### Primer Selection

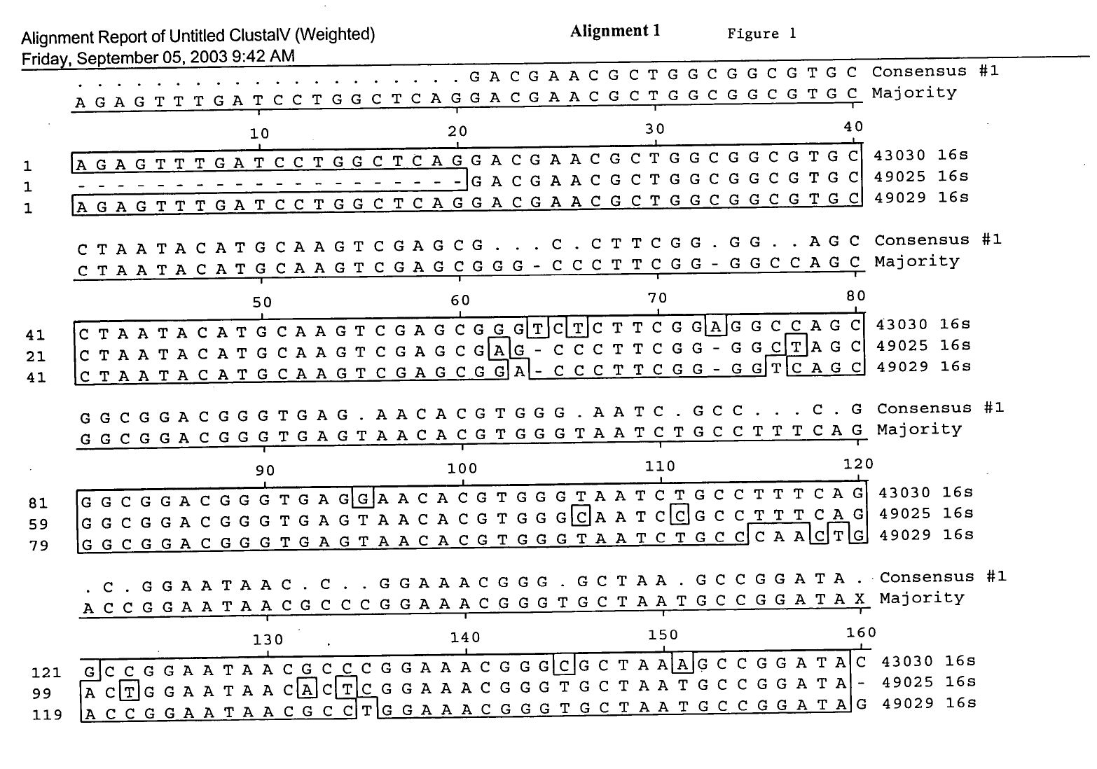

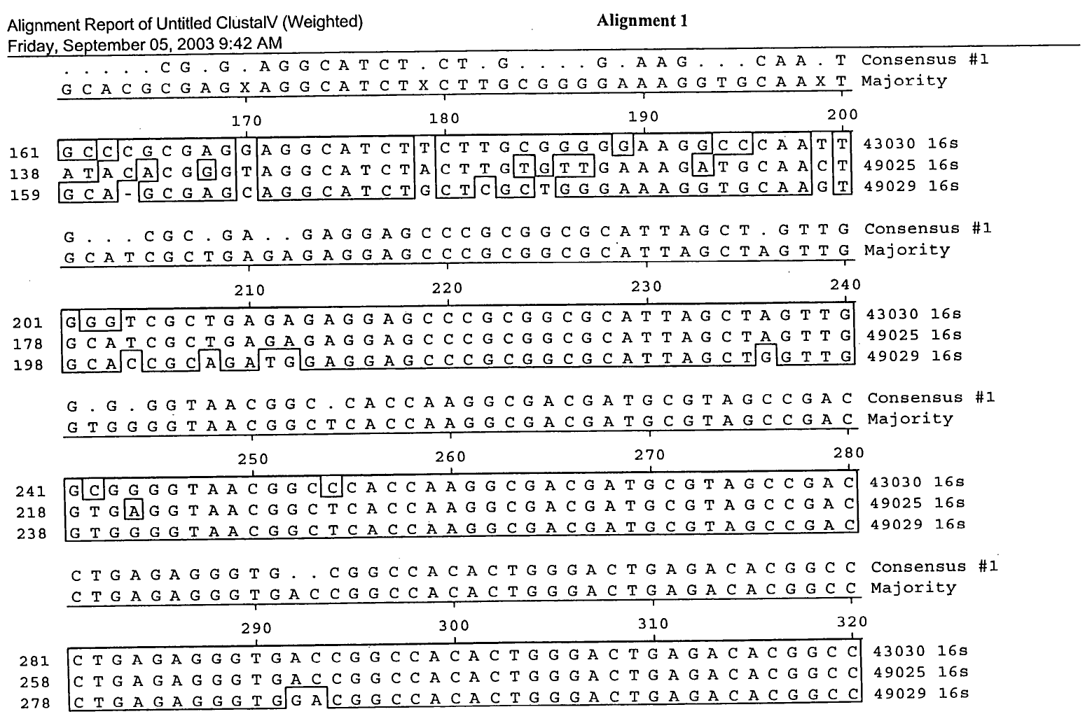

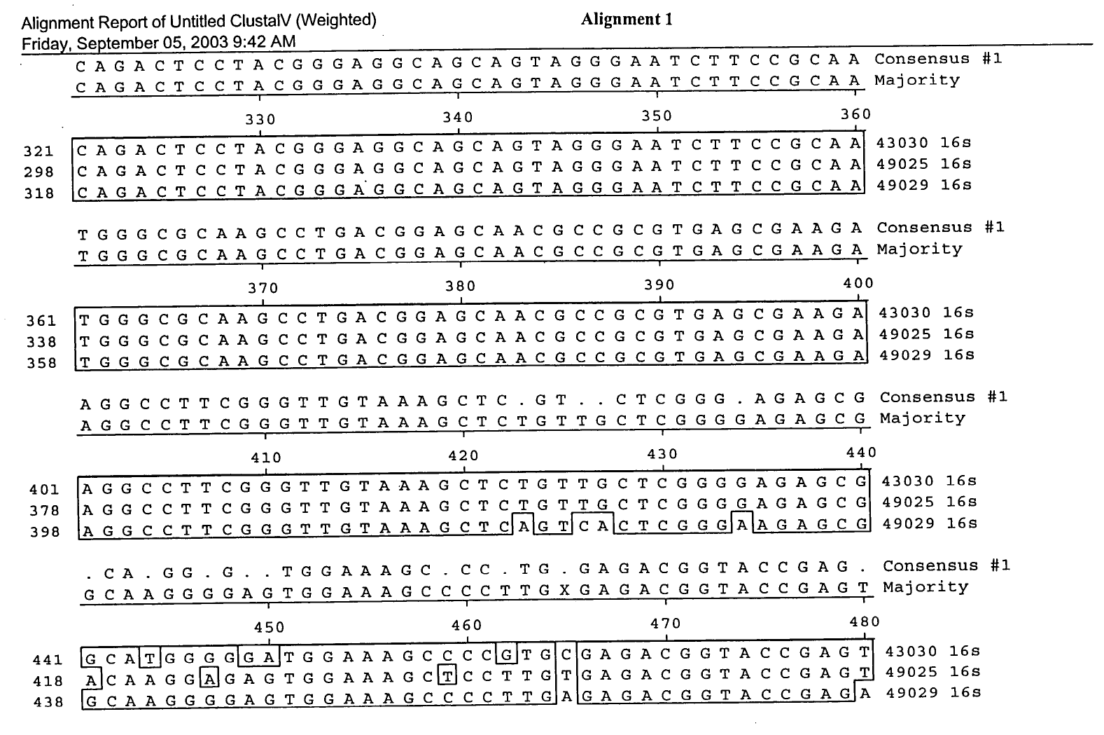

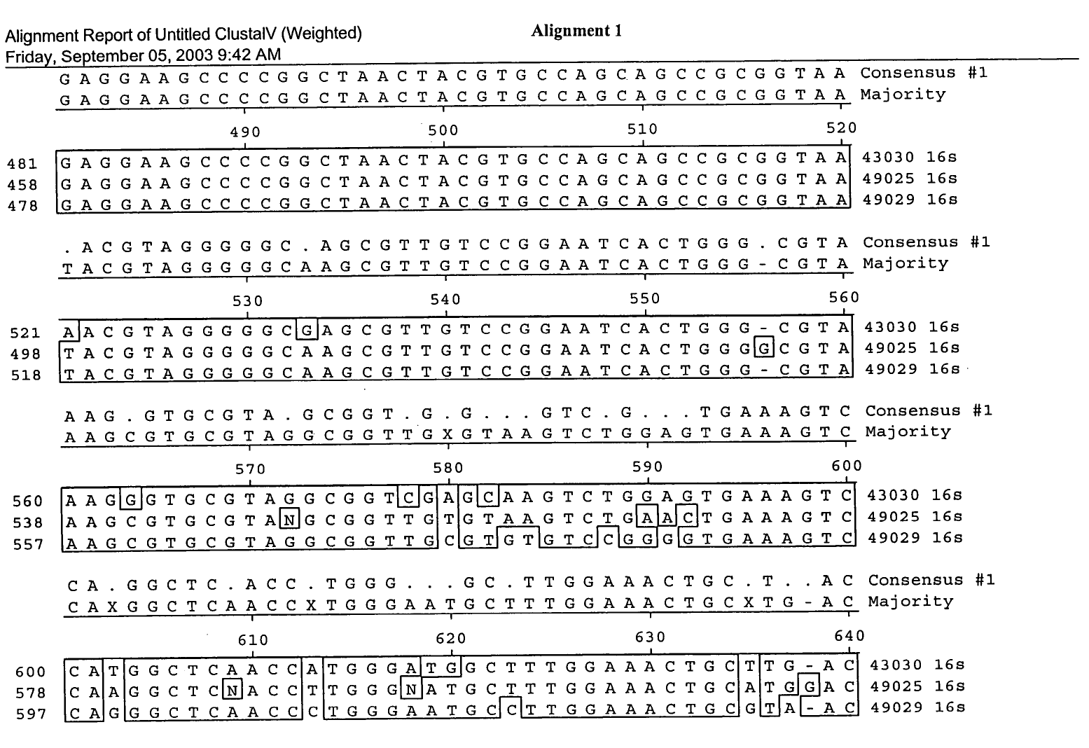

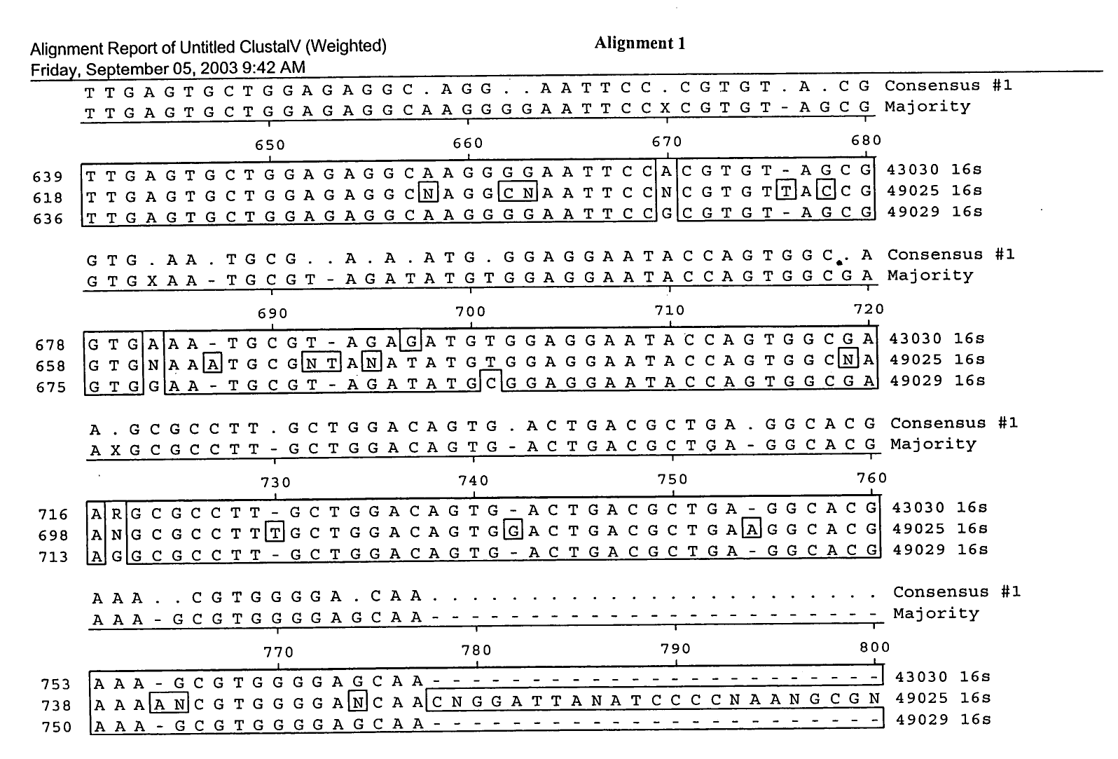

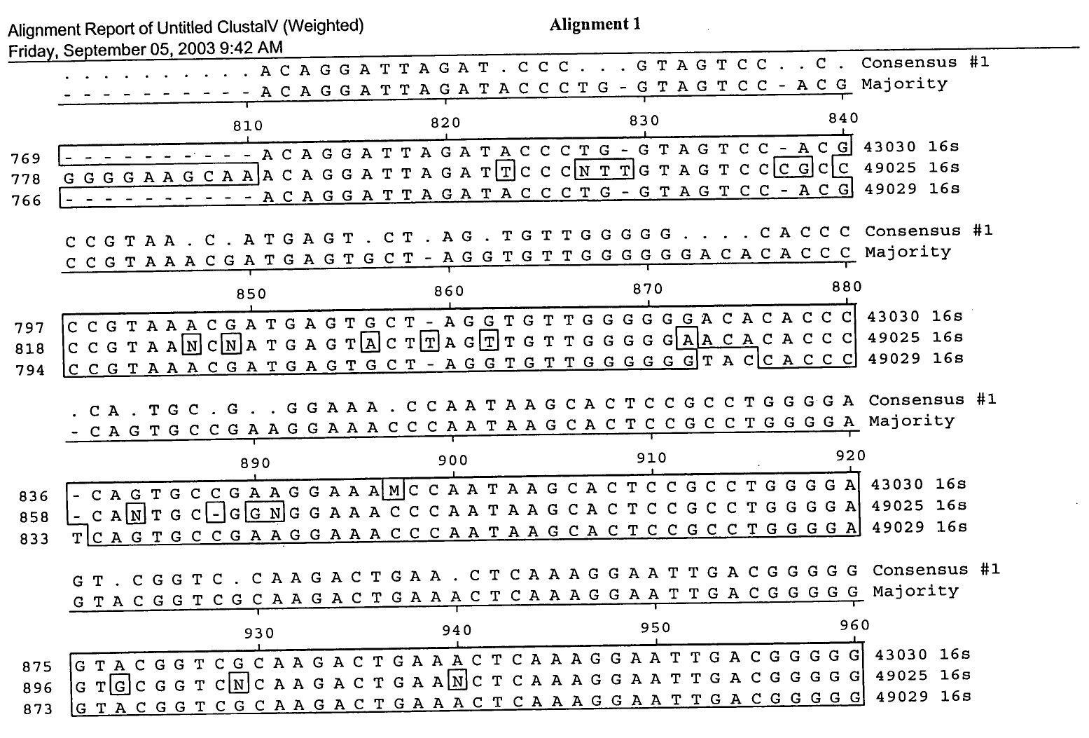

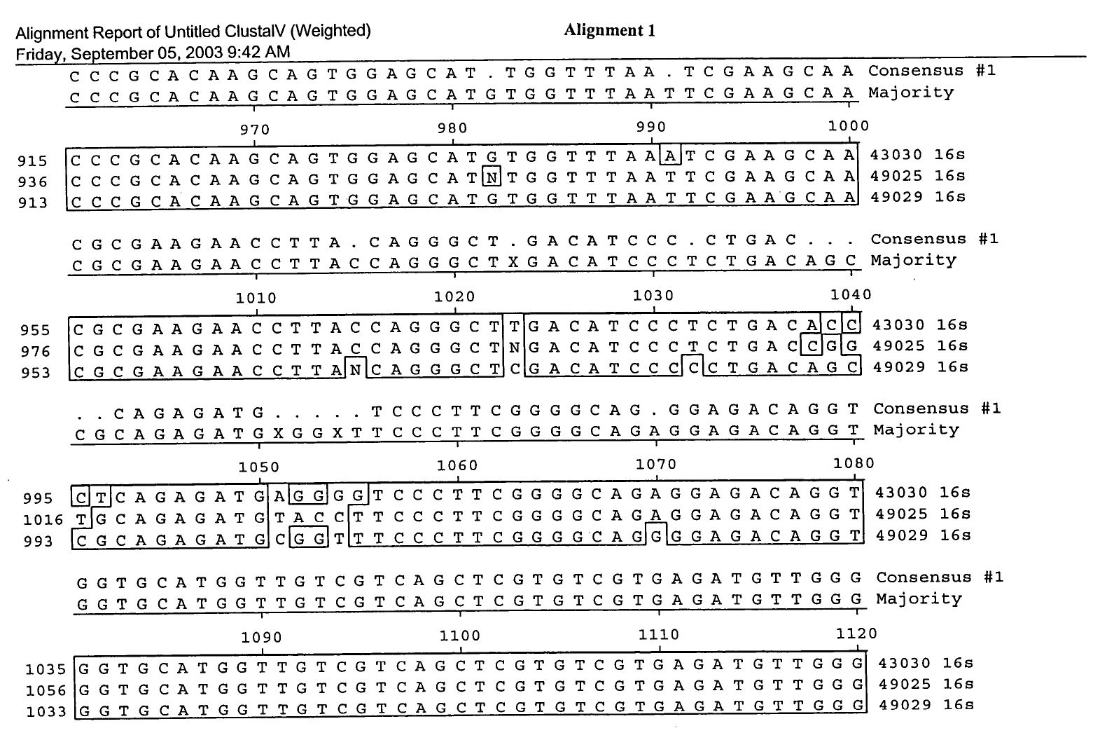

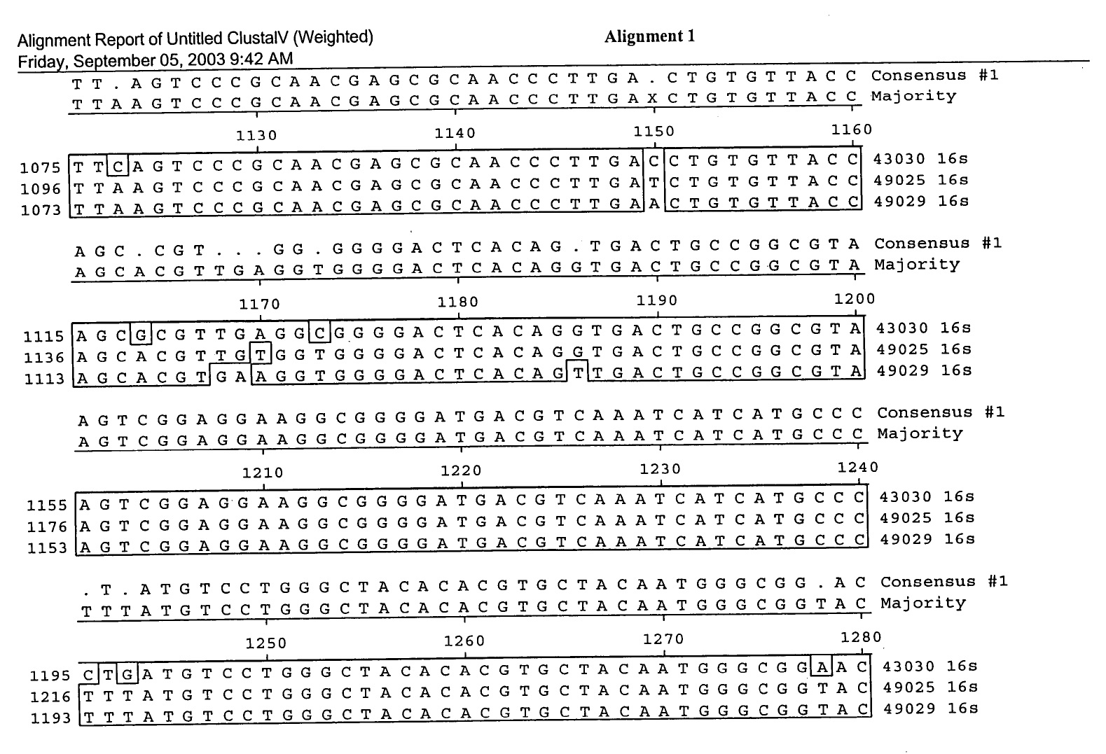

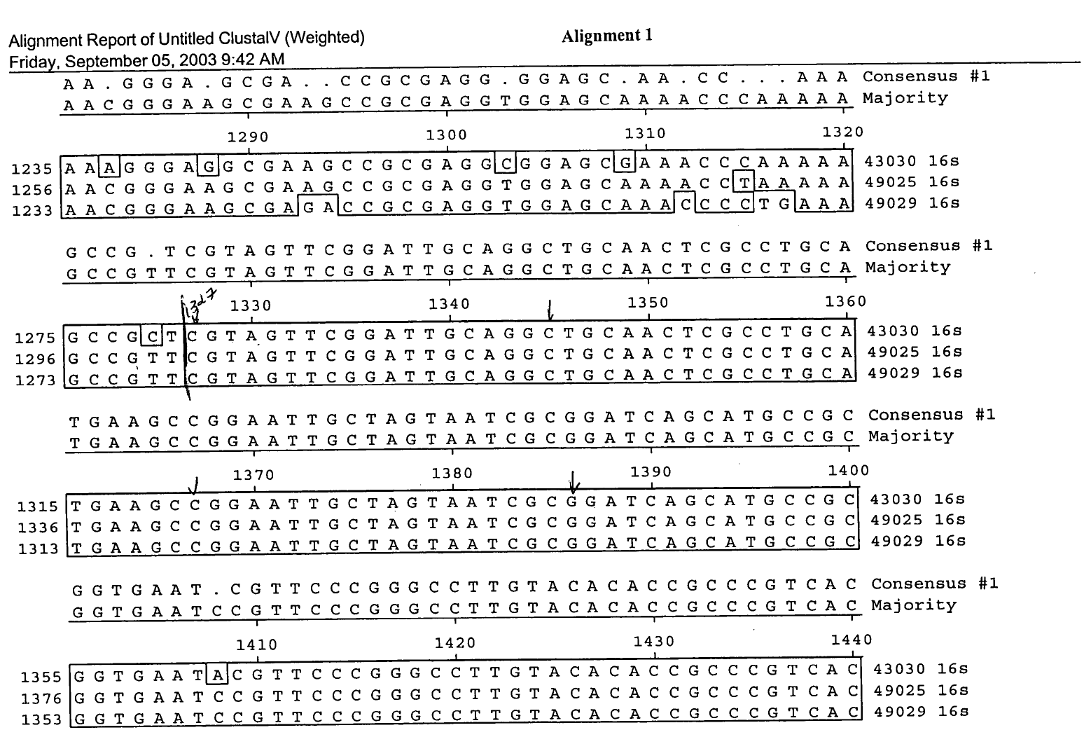

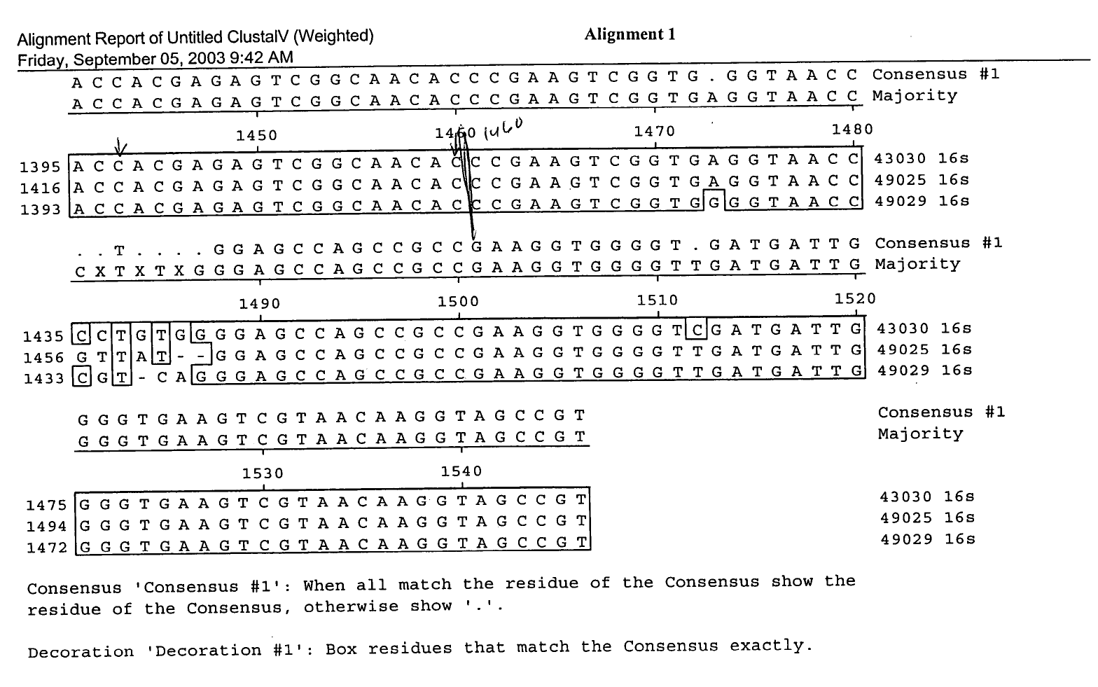

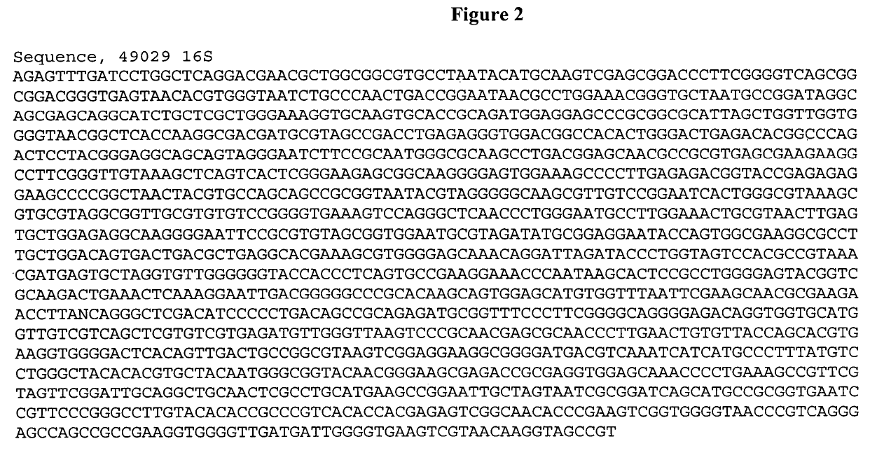

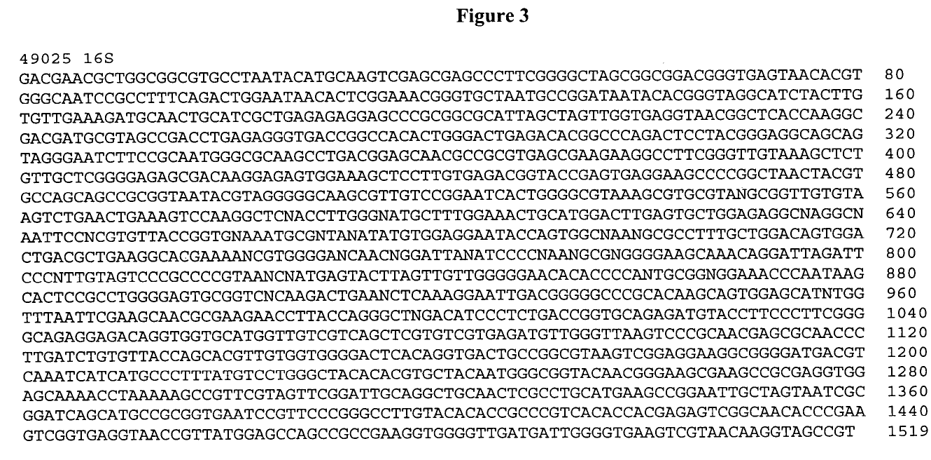

Primers are selected within the conserved regions shown in the attached alignment (FIG. 1) to amplify a fragment with proper size for optimal detection. One primer is located at each end of the sequence to be amplified. Such primers will normally be between 10 to 35 nucleotides in length and have a preferred length from between 18 to 22 nucleotides. The smallest sequence that can be amplified is approximately 50 nucleotides in length (e.g., a forward and reverse primer, both of 20 nucleotides in length, whose location in the sequences is separated by at least 10 nucleotides). Much longer sequences can be amplified. Preferably, the length of sequence amplified is between 75 and 250 nucleotides in length, and between 75 and 150 for Taqman assay.

One primer is called the “forward primer” and is located at the left end of the region to be amplified. The forward primer is identical in sequence to a region in the top strand of the DNA (when a double-stranded DNA is pictured using the convention where the top strand is shown with polarity in the 5′ to 3′ direction). The sequence of the forward primer is such that it hybridizes to the strand of the DNA which is complementary to the top strand of DNA.

The other primer is called the “reverse primer” and is located at the right end of the region to be amplified. The sequence of the reverse primer is such that it is complementary in sequence to, i.e., it is the reverse complement of a sequence in, a region in the top strand of the DNA. The reverse primer hybridizes to the top strand of the DNA.

PCR primers should also be chosen subject to a number of other conditions. PCR primers should be long enough (preferably 10 to 30 nucleotides in length) to minimize hybridization to greater than one region in the template. Primers with long runs of a single base should be avoided, if possible. Primers should preferably have a percent G+C content of between 40 and 60%. If possible, the percent G+C content of the 3′ end of the primer should be higher than the percent G+C content of the 5′ end of the primer. Primers should not contain sequences that can hybridize to another sequence within the primer (i.e., palindromes). Two primers used in the same PCR reaction should not be able to hybridize to one another. Although PCR primers are preferably chosen subject to the recommendations above, it is not necessary that the primers conform to these conditions. Other primers may work, but have a lower chance of yielding good results.

PCR primers that can be used to amplify DNA within a given sequence can be chosen using one of a number of computer programs that are available. Such programs choose primers that are optimum for amplification of a given sequence (i.e., such programs choose primers subject to the conditions stated above, plus other conditions that may maximize the functionality of PCR primers). One computer program is the Genetics Computer Group (GCG recently became Accelrys) analysis package which has a routine for selection of PCR primers. There are also several web sites that can be used to select optimal PCR primers to amplify an input sequence. One such web site is http://alces.med.umn.edu/rawprimer.html. Another such web site is http://www-genome.wi.mit.edu/cgi-bin/primer/primer3_www.cgi.

### Making the Oligonucleotide Primers and Probes

The oligonucleotide primers and probes disclosed in this application can be made in a number of ways. One way to make these oligonucleotides is to synthesize them using a commercially-available nucleic acid synthesizer. A variety of such synthesizers exists and is well known to those skilled in the art. Many such synthesizers use phosphoramidite chemistry, although other chemistries can be used. Phosphoramidite chemistry utilizes DNA phosphoramidite nucleosides, commonly called monomers, to synthesize the DNA chain or oligonucleotide. Such monomers are modified with a dimethoxytrityl (DMT) protecting group on the 5′-end, a b-cyanoethyl protected 3′-phosphite group, and may also include additional modifiers that serve to protect reactive primary amines in the heterocyclic ring structure (to prevent branching or other undesirable side reactions from occurring during synthesis).

To make an oligonucleotide of a specific sequence, phosphoramidite nucleosides are added one-by-one in the 3′-5′ direction of the oligonucleotide, starting with a column containing the 3′ nucleoside temporarily immobilized on a solid support. Synthesis initiates with cleavage of the 5′-trityl group of the immobilized 3′ nucleoside by brief treatment with acid [dichloroacetic acid (DCA) or trichloroacetic acid (TCA) in dichloromethane (DCM)] to yield a reactive 5′-hydroxyl group. The next monomer, activated by tetrazole, is coupled to the available 5′-hydroxyl and the resulting phosphite linkage is oxidized to phosphate by treatment with iodine (in a THF/pyridine/H₂O solution). The above describes the addition of one base to the oligonucleotide. Additional cycles are performed for each base that is added. The final oligonucleotide added does not have a 5′ phosphate. When synthesis is complete, the oligonucleotide is released from the support by ammonium hydroxide and deprotected (removal of blocking groups on nucleotides).

Normally, oligonucleotides of up to 150-180 bases long can be efficiently synthesized by this method using a nucleic acid synthesizer. To make oligonucleotide that are longer than 100 bases, two single-stranded oligonucleotides, that are partially complementary along their length, can be synthesized, annealed to one another to form a duplex, and then ligated into a plasmid vector. Once a plasmid containing the ligated duplexes has been formed, additional oligonucleotide duplexes can be ligated into the plasmid, adjacent to the previously ligated duplexes, to form longer sequences. It is also possible to sequentially ligate oligonucleotide duplexes to each other, to form a long, specific sequence, and then clone the single long sequence into a plasmid vector.

### Sample Preparation Flow Chart for Bacteria Detection

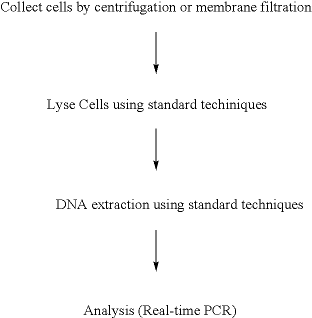

### Sample Preparation Flow Chart for Fungi (Yeast and Mold) Detection

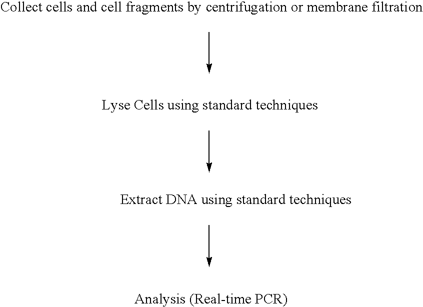

Isolation of DNA from Samples

DNA is isolated or extracted from the microorganism cells contained within the test sample. For example, DNA extraction may be performed using any of numerous commercially available kits for such purpose. One such kit, called IsoCode, is available from Schleicher and Schuell of Keene, N.H. The IsoCode kit contains paper filters onto which cells are applied. Through treatment of the paper filters as described by the manufacturer, most cellular components remain in the paper filter and DNA is released into an aqueous solution. The DNA in the solution can then be added to various enzymatic amplification reactions, as are discussed below.

Other commercially available kits exist for extraction of DNA from cells. Commercial kits do not have to be used, however, in order to obtain satisfactory DNA. Standard methods, well known to those skilled in the art, have been published in the scientific literature. Such methods commonly involve lysis of cells and removal of cellular components other than nucleic acids by precipitation or by extraction with organic solvents. Enzymatic treatment with proteases and ribonucleases can be used to remove proteins and RNA, respectively. DNA is then commonly precipitated from the sample using alcohol.

### Real-Time PCR

A variety of methods can be used to determine if a PCR product has been produced. One way to determine if a PCR product has been produced in the reaction is to analyze a portion of the PCR reaction by agarose gel electrophoresis. For example, a horizontal agarose gel of from 0.6 to 2.0% agarose is made and a portion of the PCR reaction mixture is electrophoresed through the agarose gel. After electrophoresis, the gel is stained with ethidium bromide. PCR products are visible when the gel is viewed during illumination with ultraviolet light. By comparison to standardized size markers, it is determined if the PCR product is of the correct expected size.

The PCR procedure preferably is done in such a way that the amount of PCR products can be quantified. Such “quantitative PCR” procedures normally involve comparisons of the amount of PCR product produced in different PCR reactions. A number of such quantitative PCR procedures, and variations thereof, are well known to those skilled in the art. One inherent property of such procedures, however, is the ability to determine relative amounts of a sequence of interest within the template that is amplified in the PCR reaction.

One particularly preferred method of quantitative PCR used to quantify copy numbers of sequences within the PCR template is a modification of the standard PCR called “real-time PCR.” Real-time PCR utilizes a thermal cycler (i.e., an instrument that provides the temperature changes necessary for the PCR reaction to occur) that incorporates a fluorimeter (i.e. an instrument that measures fluorescence). In one type of real-time PCR, the reaction mixture also contains a reagent whose incorporation into a PCR product can be quantified and whose quantification is indicative of copy number of that sequence in the template. One such reagent is a fluorescent dye, called SYBR Green I (Molecular Probes, Inc.; Eugene, Oreg.) that preferentially binds double-stranded DNA and whose fluorescence is greatly enhanced by binding of double-stranded DNA. When a PCR reaction is performed in the presence of SYBR Green I, resulting DNA products bind SYBR Green I and fluoresce. The fluorescence is detected and quantified by the fluorimeter. Such technique is particularly useful for quantification of the amount of template in a PCR reaction.

A preferred variation of real-time PCR is TaqMan® (Applied Biosystems) PCR. The basis for this method is to continuously measure PCR product accumulation using a dual-labeled fluorogenic oligonucleotide probe called a TaqMan® probe. The “probe” is added to and used in the PCR reaction in addition to the two primers. This probe is composed of a short (ca. 15-30 bases) oligodeoxynucleotide sequence that hybridizes to one of the strands that are made during the PCR reaction. That is, the oligonucleotide probe sequence is homologous to an internal target sequence present in the PCR amplicon. The probe is labeled or tagged with two different fluorescent dyes. On the 5′ terminus is a “reporter dye” and on the 3′ terminus is a “quenching dye.” One reporter dye that is used is called 6-carboxy fluorescein (FAM). One quenching dye that is used is called 6-carboxy tetramethyl-rhodamine (TAMRA). When the probe is intact, energy transfer occurs between the two fluorochromes and emission from the reporter is quenched by the quencher, resulting in low, background fluorescence. During the extension phase of PCR, the probe is cleaved by the 5′ nuclease activity of Taq polymerase, thereby releasing the reporter from the oligonucleotide-quencher and producing an increase in reporter emission intensity. During the entire amplification process the light emission increases exponentially.

Because the detection in Taqman assay is based on complementary binding of the third oligonucleotide probe to the amplified PCR products, it can significantly minimize false positive results due to the detection of non-specific amplification and primer dimers in conventional PCR and other non-specific real-time PCR product detection approaches such as using SYBR Green or EtBr. However, the determination of proper primer and probe set needs more specified skills so that they will fit the product amplification and signal detection requirements.

Examples of primers and probes that are particularly useful in this procedure are listed above.

### Fluorescence Detection

One example of an instrument that can be used to detect the fluorescence is an ABI Prism 7700, which uses fiber optic systems that connect to each well in a 96-well PCR tray format. The laser light source excites each well and a CCD camera measures the fluorescence spectrum and intensity from each well to generate real-time data during PCR amplification. The ABI 7700 Prism software examines the fluorescence intensity of reporter and quencher dyes and calculates the increase in normalized reporter emission intensity over the course of the amplification. The results are then plotted versus time, represented by cycle number, to produce a continuous measure of PCR amplification. To provide precise quantification of initial target in each PCR reaction, the amplification plot is examined at a point during the early log phase of product accumulation. This is accomplished by assigning a fluorescence threshold above background and determining the time point at which each sample's amplification plot reaches the threshold (defined as the threshold cycle number or CT). Differences in threshold cycle number are used to quantify the relative amount of PCR target contained within each tube.

### Detecting Fungi in Samples

### Oligonucleotide Primer and Probe Development for Detecting Yeast

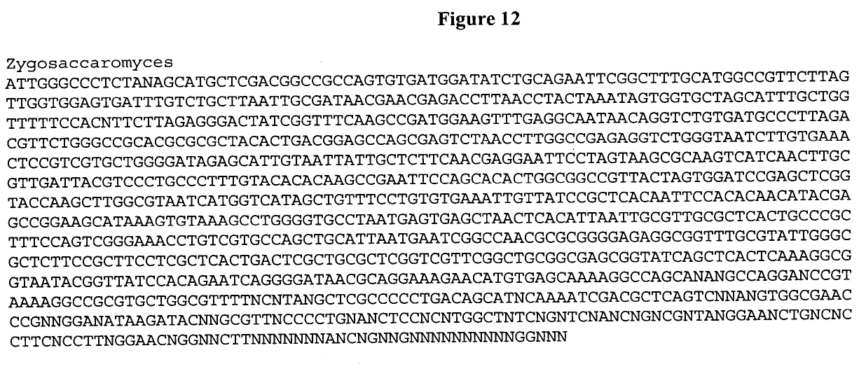

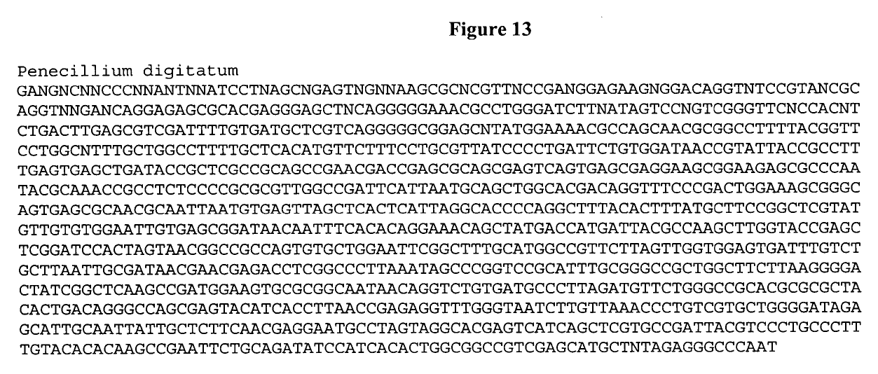

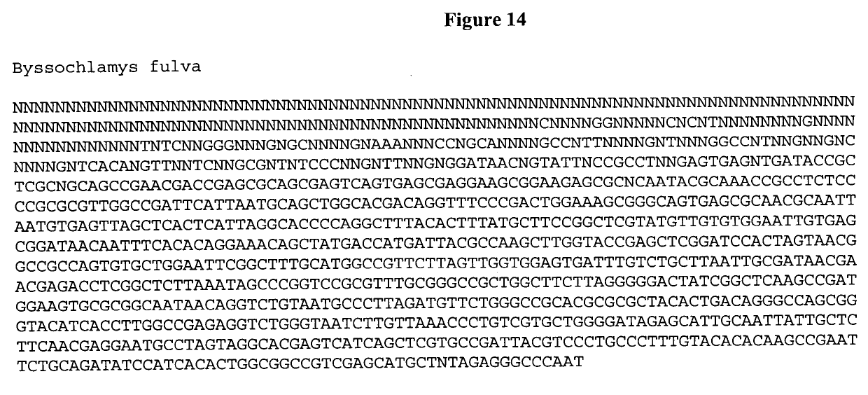

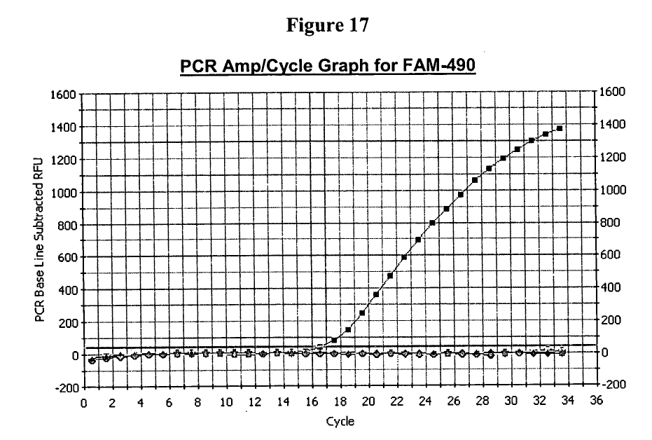

We have cloned and sequenced the 18s rDNA gene fragments from representative yeast Zygosaccharomyces bailii (Lindner) Guilliermond strain ATCC 36947. We then compared our sequences against other published 18S rDNA sequences from molds, yeasts, and common eukarytic foods. We have also compared other target sequences including h1, h2, 23S rDNA, spacer sequence between 18S and 23S rDNA gene. We have developed primer-and-probe sequences that can detect the presence of generally all yeasts without cross-reacting with foods, molds or other bacteria. The aligned sequences of the 18S rDNA sequences of these yeast species are shown in FIG. 17. FIGS. 12, 13 and 14 show the full coding sequences for the genes corresponding to the alignments shown in FIG. 17.

### Specificity Testing

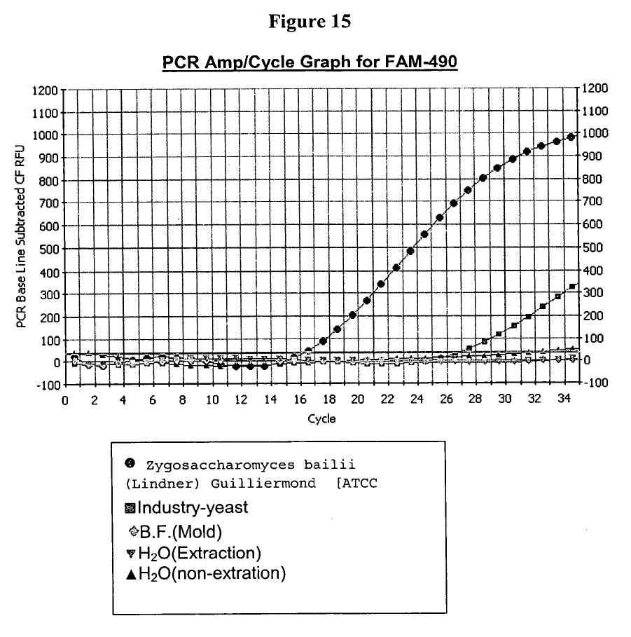

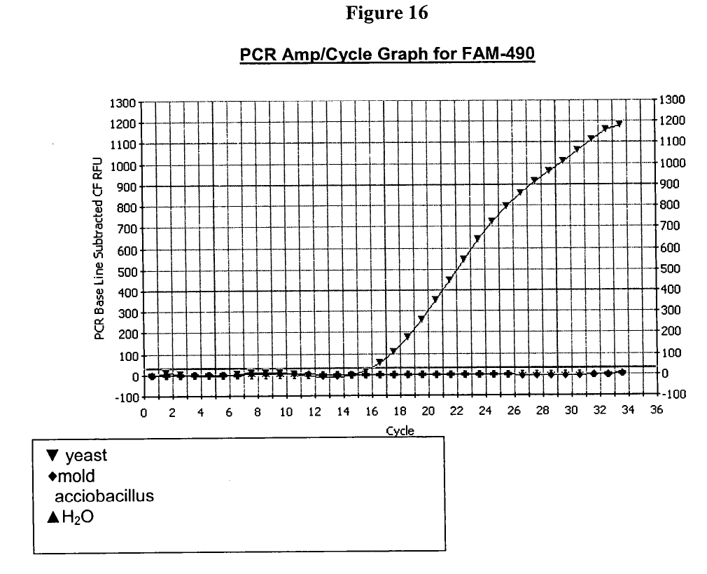

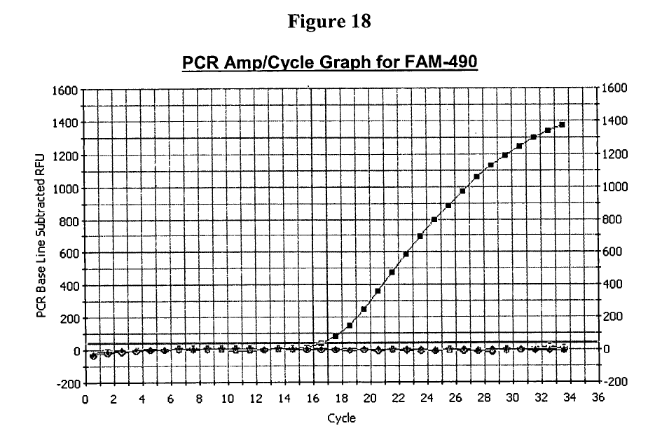

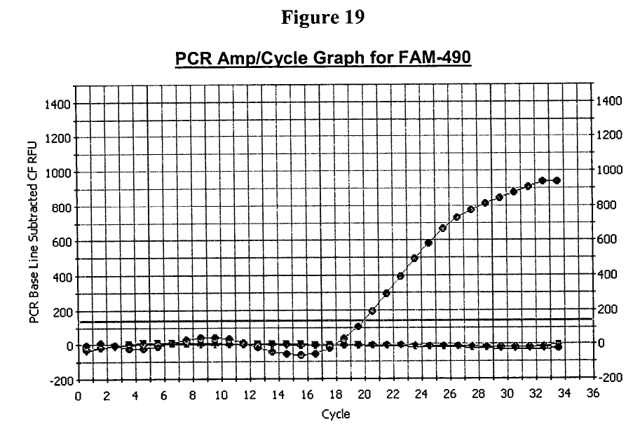

Using the primer pair-and-probe set, all yeasts were tested positive in real-time PCR (FIGS. 15-19), while no cross-reaction was detected in other commonly found foodborne microorganisms and food items (FIGS. 15-19). Further specificity study revealed no combination of the above three oligonucleotides in other microorganisms after blast searching the nucleotide sequence database in the GenBank.

### Oligonucleotide Primer and Probe Development for Detecting Mold

We have cloned and sequenced the 18s rDNA gene fragments of representative molds of food industry concerns, Byssochlamys fulva Olliver et Smith, teleomorph ATCC 24474 and Penicillium digitatum Saccardo, anamorph ATCC10030. Coloning primer up:TGCATGGCCGTTCTTAGTTGG(Z.B. code 64-75) (B.F. 667-688) (P.D. 674-695) down: GTGTGTACAAAGGGCAGGG(Z.B. 417-237) (B.F. 1011-1031) (P.D. 1029-1049). We then compared our sequences against other published 18S rDNA sequences from molds, yeasts, and common eukarytic foods. We have also compared other target sequences including h1, h2, 23S rDNA, spacer sequence between 18S and 23S rDNA gene. We have developed primer and probe sequences that can detect the presence of generally all mold without cross-reacting with foods, yeast or bacteria.

### Specificity Test

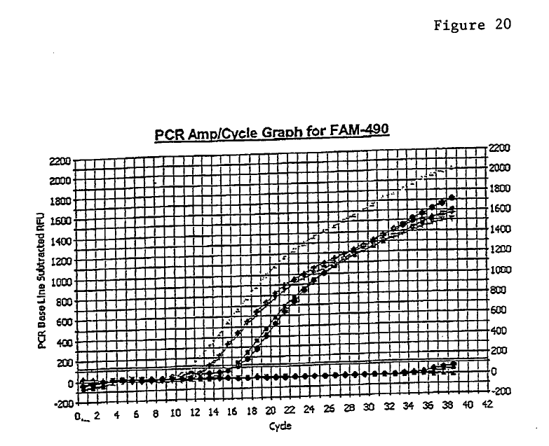

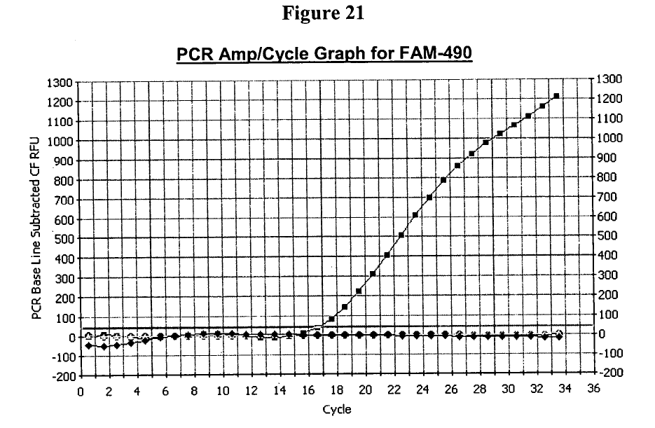

Using primer pair-and-probe set, all yeasts were tested positive in real-time PCR (FIGS. 20 and 21), while no cross-reaction was detected in other commonly found foodborne microorganisms and food items (FIGS. 20 and 21). Further specificity study revealed no combination of the above three oligonucleotides in other microorganisms after blast searching the nucleotide sequence database in the GenBank.

### REFERENCES

A. Ramesh, B P Padmapriya, A Chandrashekar, and M C Varadaraj. 2002. Application of a convenient DNA extraction method and multiplex PCR for the direct detection of Staphylococcus aureus and Yersinia enterocolitica in milk samples. Molecular and Cellular Probes. 16: 307-314.
Albuquerque L, F A Rainey, A P Chung, A Sunna, M F Nobre, R Grote, G Antranikian, and MS da Costa. 2000. Alicyclobacillus hesperidum sp. nov. and a related genomic species from solfataric soils of Sao Miguel in the Azores. Int. J. Syst. Evol. Micro. 50:451-457.
Allmann M, C Höfelein, E Köppel, J Lüthy, R Meyer, C Niederhauser, B Wegmüller, and U Candrian. 1995. Polymerase chain reaction (PCR) for detection of pathogenic microorganisms in bacteriological monitoring of dairy products. Res. Microbiology. 146: 85-97.
Asakura, A., Hoshino, T. and Shinjoh, M. 2002. Microbial production of 1-ascorbic acid and d-erythorbic acid. Patent: EP 1182262-A 2 27-Feb.-2002; Roche Vitamins AG (CH)
Bassler H. A., S. J. Flood, K. J. Livak, J. Marmaro, R. Knorr, C. A. Batt. 1995. Use of a fluorogenic probe in a PCR-based assay for the detection of Listeria monocytogenes. Appl Environ Microbiol. 61:3724-3728.
Baumgart, J., M. Husemann, and C. Schmidt. 1997. Alycyclobacillus acidoterrestris: occurrence, importance, and detection in beverages and raw materials for beverages. Flussiges Obst. 64, 178-180.
Borlinghaus A. and R Engel. 1997. Alicyclobacillus incidence in commercial apple juice concentrate (AJC) supplies—Method development and validation. Fruit Processing 7: 262-266.
Cerny G, W Hennlich, K Poralla. 1984. Spoilage of fruit juice by Bacilli: isolation and characterization of the spoilage organism. Z. Lebensm. Unters. Forsch. 179:224-227.
Danbing K, C Menard, F J Picard, M Boissinot, M Ouellette, P H Roy, and M G Bergeron. 2000. Development of conventional and real-time PCR Assays for the rapid detection of Group B Streptococci. Clinical Chemistry. 46:324-331.
Darland G. and Brock T. D. (1971) Bacillus acidocaldarius sp.nov., an acidophilic thermophilic spore-forming bacterium. Journal of General Microbiology 67, 9-15
D M Whiley, G M LeCornecc, I M Mackay, D J. Siebertc, and T P Slootsa. 2002. A real-time PCR assay for the detection of Neisseria gonorrhoeae by LightCycler. Diagnostic Microbiology and Infectious Disease. 42: 85-89.
D'Souza D H, Jaykus L A. 2003. Nucleic acid sequence based amplification for the rapid and sensitive detection of Salmonella enterica from foods. J Appl Microbiol. 95(6):1343-50.
Evancho G M, and I Walls. 2001. Aciduric Flat Sour Sporeformers. Compendium of Methods for the Microbiological Examination of Foods. 239-244. 4^{th} edition.
Evangelia, K., S. B. Ioannis, E. D. Alison, D. B. Joss, and R. A. Martin. 1999. Alicyclobacillus acidoterrestris in fruit juices and its control by nisin. Int. J. Food Sci. Technol. 34:81-85.
Eyigor A, K T Carli, and C B Unal. 2002. Implementation of real-time PCR to tetrathionate broth enrichment step of Salmonella detection in poultry. Letters in App. Micro. 34: 37-41.
Goto K, H Matsubara, K Mochida, T Matsumura, Y Hara, M Niwa, and K Yamasato. 2002. Alicyclobacillus herbarius sp. nov., a novel bacterium with ω-cycloheptane fatty acids, isolated from herbal tea. Int. J. Syst. Evol. Micro. 52: 109-113.
Goto K., K. M. Mochida, M. Asahara, M. Suzuki and Yokota A(2002) Application of the hypervariable region of the 16S rDNA sequence as an index for the rapid identification of species in the genus Alicyclobacillus. J Gen Appl Microbio 48, 243-50 Matsubara, K. 2003. A method for detecting and/or identifying guaiacol producing microorganism. Patent: JP 2003000259-A 8.
Goto, K, K Mochida, M Asahara, M Suzuki, H Kasai and A. Yokota. 2003. Alicyclobacillus pomorum sp. nov., a novel thermo-acidophilic, endospore-forming bacterium that does not possess omega-alicyclic fatty acids, and amended description of the genus Alicyclobacillus. Int. J. Syst. Evol. Micro. Papers in Press
Goto, K, Y Tanimoto, T Tamura, K Mochida, D Arai, M Asahara, M Suzuki, H Tanaka, and K Inagki. 2002. Identification of thermophilic bacteria and a new Alicyclobacillus genomic species isolated from acidic environments in Japan. Extremeophiles. 6: 333-340.
Haugland R A, S J Vesper and L J Wymer. 1999. Quantitative measurement of Stachybotrys chartarum conidia using real time detection of PCR products with the TaqMan fluorogenic probe system. Molecular and Cellular Probes. 13: 329-340.
Heid C A, J Stevens, K J Livak, and P M Williams. 1996. Real time quantitative PCR. Genome Res. 10: 986-94.
Heid C A. Real-time quantitative PCR. Genome Res 6, 986-994
Hippchen, B., A. Roll, and K. Poralla. 1981. Occurrence in soil of thermo-acidophilic Bacilli possessing ω-cyclohexane fatty acids and hopanoids. 129:53-55.
Ibekwe A. M., P. M. Watt, C. M. Grieve, V. K. Sharma, and Lyons S. R. (2002) Multiplex fluorogenic real-time PCR for detection and quantification of Escherichia coli O157:H7 in dairy wastewater wetlands. Appl. Environ. Microbiol. 68, 4853-4862
Jensen N, and F B Whitfield. 2003. Role of Alicyclobacillus acidoterrestris in the development of a disinfectant taint in shelf-stable fruit juice. Letters. App. Micro. 36: 9-14.
Kaiser K, M Rabodonirina, and S Picot. 2001. Real time quantitative PCR and RT-PCR for analysis of Pneumocystis carinii hominis. J. of Micro. Methods. 45: 13-118.
Kannenberg E. L. and Poralla K. (1999) Hopanoid biosynthesis and function in bacteria. Naturwissenschaften 86, 168-176
Koo K, Jaykus L A. 2002. Detection of single nucleotide polymorphisms within the Listeria genus using an ‘asymmetric’ fluorogenic probe set and fluorescence resonance energy transfer based-PCR. Lett Appl Microbiol. 35(6):513-7. Koo K, Jaykus L A. 2003. Detection of Listeria monocytogenes from a model food by fluorescence resonance energy transfer-based PCR with an asymmetric fluorogenic probe set. Appl Environ Microbiol. 69(2): 1082-8.
Lee S Y, Dougherty R H, Kang D H. 2002. Inhibitory Effects of High Pressure and Heat on Alicyclobacillus acidoterrestris Spores in Apple Juice. Appl. Environ. Microbiol. 68:4158-4161.
Livak K J, Flood S J, Marmaro J, Giusti W, Deetz K. 1995. Oligonucleotides with fluorescent dyes at opposite ends provide a quenched probe system useful for detecting PCR product and nucleic acid hybridization. PCR Methods Appl. 4:357-362.
Makino S I, H I Cheun, M Watarai, I Uchida and K Takeshi. 2001. Detection of anthrax spores from the air by real-time PCR. Letters in Applied Microbiology. 33: 237-240.
Matsubara H, K Goto, T Matsumura, K Mochida, M Iwaki, M Niwa, and K Yamasato. 2002. Alicyclobacillus acidiphilus sp. nov., a new thermo-acidophilic, w-alicyclic fatty acid-containing bacterium isolated from acidic beverages. Int. J. Syst. Evol. Micro. Papers in press. http://dx.doi.org/10.1099/ijs.0.02169-0
McIntyre, S, J Y Ikawa, N Parkinson, J Haglund, and J Lee. 1994. Characteristics of an acidophilic
McSpadden-Gardener B B, D V Mavrodi, L S Thomashow, and DM Weller. 2001. A rapid polymerase chain reaction-based assay characterizing Rhizosphere populations of 2,4-diacetylphloroglucinol-producing bacteria. Phytopathology. 91: 44-54.
Obara, A. and Niwa, M. 1998. Nucleic acid coding enzyme participating to biosynthesis of omega-cyclohexane fatty acid, nucleic acid primer containing part or whole of base sequence of the nucleic acid, and detection and identification of microorganisms belonging to genus Alicyclobacillus. Patent: JP 1998234376-A 7 08-Sep.-1998
Ochs, D., C. Kaletta, K. Entian, A. Beck-Sickinger, and K. Poralla. 1992. Cloning, expression, and sequencing of squalene-hopene cyclase, a key enzyme in triterpenoid metabolism. J. Bacteriol. 174:298-302.
Orr, R. V., L. Robert, C. J. H. Shewfelt, T. Sebhat, and L. Y. R. Beuchat. 2000. Detection of guaiacol produced by Alicyclobacillus acidoterrestris in apple juice by sensory and chromatographic analyses, and comparison with spore and vegetative cell populations. J. Food Prot. 63:1517-1522.
Pettipher G L, M E Osmundson, and J M Murphy. 1997. Methods for detection and enumeration of Alicyclobacillus acidoterrestris and investigation of growth and production of taint in fruit juice and fruit juice-containing drinks. Letters in App. Micro. 24: 185-189.
Pinhatti M E M C, S Variane, S Y Eguchi, and G P Manifo. 1997. Detection of acidothermophilic Bacilli in industrialized fruit juices. Fruit Processing. 7: 350-353.
Previdi M P, S Quintavalla, C Lusardi, and E Vincini. 1997. Heat resistance of Alicyclobacillus spores in fruit juices. Indust. Conserve. 72: 353-358.
Shearer A E, Dunne C P, Sikes A, Hoover D G. 2002. Bacterial spore inhibition and inactivation in foods by pressure, chemical preservatives, and mild heat. J Food Prot. 63:1503-1510. Silva, F. V. M. and P. Gibbs. 2001. Alicyclobacillus acidoterrestris spores in fruit products and design of pasteurization processes. Trends Food Sci. and Tech. 12:68-74.
Silva F V M, and P Gibbs. 2001. Alicyclobacillus acidoterrestris spores in fruit products and design of pasteurization process. Trends in Food Sci. & Tech. 12:68-74.
Silva F V M, P Gibbs, M C Vieira, and C L M Silva. 1999. Thermal inactivation of Alicyclobacillus acidoterrestris spores under different temperature, soluble solids, and pH conditions for the design of fruit processes. Intl. J. of Food Micro. 51: 95-103.
Splittstoesser D F, J J Churey, and C Y Lee. 1994. Growth characteristics of aciduric sporeforming Bacilli isolated from fruit juices. J. Food Prot. 57: 1080-1083.
Splittstoesser, D. F., C. Y. Lee, and J. J. Churry. 1998. Control of Alicyclobacillus in the juice industry. Dairy Food Environ. Sanit. 18:585-587.
Tsuruoka N, Y Isono, O Shida, H Hemmi, T Nakayama, T Nishino. 2002. Alicyclobacillus sendiensis sp. nov., a novel acidophilic, slightly thermophilic species isolated from soil in Sendai, Japan. Int. J. Syst. Evol. Micro. Papers in Press,
Tsuruoka, N., T. Nakayama, M. Ashida, H. Hemmi, M. Nakao, H. Minakata, H. Oyama, K. Oda, and T. Nishino. 2003. Collagenolytic Serine-Carboxyl Proteinase from Alicyclobacillus sendaiensis Strain NTAP-1: Purification, Characterization, Gene Cloning, and Heterologous Expression. Appl. Environ. Microbiol. 69:162-169.
Weisburg W G, S M Barns, D A Pelletier, and D J Lane. 1991. 16S Ribosomal DNA amplification for phylogenetic study. J. of Bact. 173: 697-703.
Wendt K. U., K. Poralla., and Schulz G. E. (1997) Structure and function of a squalene cyclase. Science 277, 1811-1815
Wisotzkey, J D, P Jurtshuk, G E Fox, G Deinhard, and K Poralla. 1992. Comparative sequence analysis on the 16s rRNA of Bacillus acidocaldarius and Bacillus acidoterrestris and Bacillus cycloheptanicus and proposal for creation of new genus Alicyclobacillus gen. nov. Intl. J. of Syst. Bact. 42: 263-269.
Yamazaki K, Teduka H, Shinano H. 1996, Isolation and identification of Alicyclobacillus acidoterrestris from acidic beverages. Bioscience, Biotechnology and Biochemistry. 60: 543-545.
Yamazaki, K., H. Teduka, N. Inoue, and H. Shinano. 1996. Specific primers for detection of Alicyclobacillus acidoterrestris by RT-PCR. Lett. Appl. Microbiol. 23:350-354.

### EXAMPLES

### Example 1

In this study, the 16s rDNA sequences of A. acidocaldarius, A. cycloheptanicus, and A. acidoterrestris were used as models for the development of specific primers and a fluorogenic probe to be used in a real-time PCR assay. 16s rDNA was isolated from ATTC strains 43030, 49025, and 49029, then cloned into vectors, transformed into competent cells, and purified for sequencing. Following sequencing, the 16s rDNA sequences of the three strains were analyzed for the development of oligonucleotide primers and a fluorescent probe. These primers and probe were used in a real-time PCR detection system where specificity and sensitivity tests were performed in media as well as beverage systems. This rapid detection system is unique because it can specifically detect not only the three original Alicyclobacillus species, but also detects newer species of Alicyclobacillus because of the genus-level 16s rDNA conservation of the priming sequences. This system can greatly benefit the food industry, particularly the beverage industry, by detecting the presence of Alicyclobacillus within hours, before the product ever reaches the consumer, saving not only time and money, but maintaining brand image and quality.

### Materials and Methods

Bacterial strains and culture conditions. A. acidocaldarius strain ATTC 43030 was grown on ATCC 573 medium, consisting of 1.3 g (NH₄)₂SO₄, 0.37 g KH₂PO₄, 0.25 g MgSO₄.7H₂O, 0.07 g CaCl₂.2H₂O, 1.0 g glucose, 1.0 g yeast extract, and 1.0 L distilled H₂O, Solution pH was adjusted to 4.0 using H₂SO₄ and autoclaved at 121° C. for 15 minutes. A. acidoterrestris strain ATTC 49025 and A. cycloheptanicus strain ATCC 49029 were grown on BAM-SM ATCC 1656 medium consisting of 0.25 g CaCl₂.2H₂O, 0.5 g MgSO₄.7H₂O, 0.2 g (NH₄)₂SO₄, 3.0 g KH₂PO₄, 6.0 g yeast extract, 5.0 g glucose, 1.0 mL trace elements (0.66 g CaCl₂.2H₂O, 0.18 g ZnSO₄.7H₂O, 0.16 g CuSO₄.5H₂O, 0.15 g MnSO₄.4H₂O, 0.18 g CoCl₂.6H₂O, 0.10 g H₃BO₃, 0.30 g Na₂MoO₄.2H₂O, 1.0 L distilled H₂O), and 1.0 L distilled H₂O, Solution pH was adjusted to 4.0 using H₂SO₄ and autoclaved at 121° C. for 15 minutes. Stock cultures of all strains were stored in their respective media plus 40% glycerol and kept at −80° C.
Isolation of genomic DNA and amplification of 16s rDNA. DNA was isolated from 2% cultures of A. acidoterrestris strain ATTC 49025, A. cycloheptanicus strain ATCC 49029 A. acidocaldarius strain ATTC 43030 in respective media. Cultures were grown for 24 hours at 47° C. Genomic DNA was extracted from each strain using the Qiagen DNeasy Tissue Kit (Qiagen, Valencia, Calif.). The included protocol was followed, except the elution was repeated once with 100 μl of buffer AE. An approximately 1,500 bp region of the 16s rDNA was amplified from the genomic DNA using primers 8F and 1492R (15) with PCR performed on the Bio-Rad iCycler Thermal Cycler (Bio-Rad Laboratories, Hercules, Calif.). A 50 μl reaction mixture was used, containing 0.511 of primer 8F, 0.5 μl of primer 1492R, 1.0 μl of genomic DNA, 37 μl of sterile H₂O, 3 μl of 50 mM MgCl₂, 2 μl of a 10 mM dNTP mixture, and 1.01 Taq polymerase (Invitrogen, Carlsbad, Calif.). Amplification conditions included 30 cycles of 95° C. for 2 min, 42° C. for 30 s, and 72° C. for 4 min, with a final chain elongation for 20 min (15). PCR products were confirmed after 20 min of gel electrophoresis on 0.9% agarose gel at 100 volts, followed by 10 min of ethidium bromide staining for visualization.
Cloning and transformation of 16s rDNA gene. PCR products were purified using the QIAquick PCR purification kit (Qiagen, Valencia, Calif.). The protocol was followed as specified by the manufacturer, except 3011 of sterile H₂O was used in place of 50 μl of buffer EB for a single elution. Purified PCR products were then cloned into pCR 2.1 vectors using the TA Cloning kit (Invitrogen, Carlsbad, Calif.). A 10 μl ligation reaction for each PCR product was prepared as follows: 5 μl sterile H₂O, 1 μl pCR 2.1 vector, and 2 μl PCR product were mixed together and incubated at 65° C. for 5 min, followed by 10 min of incubation on ice. 1 μl 10× ligation buffer and 1 μl T4 DNA ligase were then added to the mixture, followed by overnight incubation at 14° C. Transformation was then performed, beginning with centrifugation of the ligation reactions. Reactions were stored on ice while 50 μl of One Shot competent Escherichia coli cells were thawed for each transfer. 5 μl of each ligation reaction was added to a vial of One Shot cells and mixed gently, followed by incubation for 30 min on ice. Reactions were then heat shocked for 30 s at 42° C., and then placed on ice. 200 μl of SOC medium was added to each tube and then shook at 200 rpm for one hour at 37° C. The whole vial of cells was then spread onto LB agar plates containing X-Gal (20 mg/ml) and incubated at 37° C. overnight. Plates were stored at 4° C. following incubation.
Sequencing of 16s rDNA gene. Plates were observed for transformed (white) colonies. Five transformed colonies from each plate were selected using a sterile toothpick, then dipped into a microfuge tube containing 100 μl of sterile H₂O, and also spread on an LB agar plate. The stick was then placed into a tube containing 2 ml of LB broth and ampicillin (50 mg/ml). Plates were incubated at 37° C. overnight. LB tubes were shaken at 100 rpm at 37° C. overnight. Microfuge tubes were incubated at 100° C. for 10 min, followed by PCR to check for successful transformation. Standard 3-step PCR (CYCLES) was run with a 50 μl reaction mixture containing 0.5 μl of primer M13F, 0.5 μl of primer M13R, 1.0 μl of transformed DNA, 37 μl of sterile H₂O, 3 μl of 50 mM MgCl₂, 211 of a 10 mM dNTP mixture, and 1.01 Taq polymerase (Invitrogen, Carslbad, Calif.). PCR products were analyzed by gel electrophoresis. LB tubes were centrifuged for 10 min at 6000 rpm after overnight incubation and used in the QIAprep Spin Miniprep kit (Qiagen, Valencia, Calif.) following the manufacturer's protocol. 5 μl of product was set aside for PCR, and the rest of the miniprep yield was sent to be sequenced. Sequence data was entered into the NCBI BLAST network to search for similar sequences. Cloned sequences from ATCC strains 49025, 49029, and 43030 matched multiple 16s rDNA sequences from Alicyclobacillus species on the BLAST network.
Real-time Tagman PCR conditions. Fifty microliter reaction mixtures containing 0.5 μl of a 100 μM solution of CC16S-F primer, 0.5 μl of a 100 μM solution of CC16S-R primer, 0.5 μl of a 100 μM solution of CC16S-Probe, 33.3 μl of sterile H₂O, 5.0 μl of genomic DNA, 5 μl of 10× reaction buffer, 3 μl of MgCl₂, 2 μl of dNTP's, and 0.2 μl of Taq polymerase (Invitrogen, Carlsbad, Calif.) were used for specificity tests. For sensitivity assays, the following 5011 reaction mixtures were used: 25 μl of 2×iQ Supermix, containing 100 mM KCl, 40 mM Tris-HCl, pH 8.4, 0.4 mM each dNTP, 50 U/ml iTaq DNA polymerase, 6 mM MgCl₂, and stabilizers (Bio-Rad, Hercules, Calif.), 0.5 μl of 100 μM stock CC16S-F primer, 0.5 μl of 100 μM stock CC16S-R primer, 0.5 μl of 100 μM stock CC16S-Probe, 5.0 μl of genomic DNA, and 18.5 μl of sterile H₂O. Real-time PCR was performed using the iCycler iQ Real-Time PCR Detection System (Bio-Rad, Hercules, Calif.). PCR conditions were as follows: 35-40 cycles of 95° C. denaturation for 30 s and 55° C. annealing for 30 s. The optical module was set to capture light during the annealing step. Results were analyzed using the iCycler iQ Optical System Software Version 3.0a (Bio-Rad, Hercules, Calif.).
Primer and probe design. Sequence alignments of the 16s rDNA sequences for strains 49025, 29029, and 43030 were constructed with ClustalV using MegAlign 5.01 (DNASTAR, Madison, Wis.). A sequence alignment of the 16S rDNA sequences was then performed for the following organisms: sequenced Alicyclobacillus strains ATCC 49025, 49029, and 43030, A. acidoterrestris strain DSM 3923 (AB042058), A. cycloheptanicus strain DSM 4006 (AB042059), A. acidocaldarius strain DSM 454 (AB059664), Geobacillus subterraneus strain K (AF276307), Sulfobacillus disulfidooxidans SD-11 (U34974), B. thermoleovorans strain ATCC 43513 (M77488), and Clostridium elmenteitii isolate E2SE1-B (AJ271453). The alignment was constructed with ClustalV using MegAlign 5.01 (DNASTAR, Madison, Wis.). Aligned regions were carefully scanned by eye to find areas of perfect identity within the representative Alicyclobacillus species in order to create PCR priming regions. The following criteria were used for primer and probe selection: (1) 100% identity between representative sequences, (2) priming region of less than 200 bp, (3) Tₘ greater than 55° C., (4) C or G in the terminal positions of both 5′ and 3′ ends, (5) greater than 45% C+G content, and (6) no visual hairpin loops or secondary structures, confirmed using the Oligo Toolkit (Qiagen, Valencia, Calif.) (22).
Specificity and sensitivity tests. Assays were performed using the aforementioned PCR conditions to test for specificity of the system for Alicyclobacillus spp. and any cross-reactions with other common food-borne microorganisms. Genomic DNA was extracted from broth cultures of 2% A. acidoterrestris, A. acidocaldarius, and A. cycloheptanicus grown for 48 h at 47° C. using the previously discussed DNA extraction protocol. In addition, genomic DNA was extracted from Escherichia coli DH-5α, Lactococcus lactis subsp. lactis, Geobacillus stearothermophilus ATCC 10149 and Pseudomonas putida 49L/51 to test specificity of the primers and probe.

Assays for the sensitivity of the real-time PCR assay for detection of Alicyclobacillus were performed using tenfold serial dilutions of 100 to 10⁻⁸ of A. acidoterrestris in a 10 ml solution of 0.85% NaCl. Two percent cultures were initially grown for 48 h at 47° C. in order to obtain an OD₆₀₀ range between 0.400 and 0.800. After dilution, cells from 1 ml of each sample were collected by centrifugation at 12,000 rpm for 10 minutes for DNA extraction. Fifty microliter (50 μl) reaction mixtures containing 0.5 μl of CC16S-F primer, 0.5 μl CC16S-R primer, 0.5 μl CC16S-Probe, 33.3 μl of sterile H₂O, 5.0 μl of genomic DNA, 5 μl of 10× reaction buffer, 3 μl of MgCl₂, 2 μl of dNTP's, and 0.2 μl of Taq polymerase (Invitrogen, Carlsbad, Calif.) were used for each strain, as described above. Real-time PCR was carried out with the following cycling conditions: 35-40 cycles of 95° C. and 55° C., for 30 s each. After amplification, results were analyzed using the iCycler iQ Optical System Software Version 3.0a (Bio-Rad, Hercules, Calif.). A range of dilutions between 10⁻³ and 10⁻⁷ were plated on BBL Orange Serum Agar (Difco, Detroit) for colony counting. Plates were incubated at 47° C. for 48 h. Additionally, sensitivity tests were performed in the same manner using apple and orange juice. Also, 1 ml of culture was spiked in 9 ml of Powerade sports drinks and Minute Maid Lemonade to check for any inhibitory characteristics these drinks may display in a PCR assay.

Amplification, cloning, transformation, and sequencing of 16s rDNA gene. PCR was used to successfully amplify regions of 16s rDNA from A. acidoterrestrs, A. acidocaldarius, and A. cycloheptanicus using the 8F and 1492R primers. The Invitrogen TA cloning kit was used to insert the amplified 16s rDNA segment of each strain into pCR 2.1 vectors, and subsequently transformed into E. coli competent cells. Purified samples were then sent to the Plant-Microbe Genomic Facility at the Ohio State University and sequenced using an ABI PRISM 3700 DNA Analyzer (Applied Biosystems, Foster City, Calif.).

TABLE V

Oligonucleotide data for Alicyclobacillus spp.
CC16S probe and primers.

| | | | | G + C |
|---|---|---|---|---|
| Name | Sequence | Length | Tₘ | content |
| CC16S-F | CGTAGTTCGGATTGCAGGC | 19 bp | 65.6° C. | 57.9% |
| CC16S-R | GTGTTGCCGACTCTCGTG | 18 bp | 63.3° C. | 61.1% |
| CC16S- | CGGAATTGCTAGTAATCGC | 19 bp | 57.9° C. | 47.4% |
| Probe | | | | |

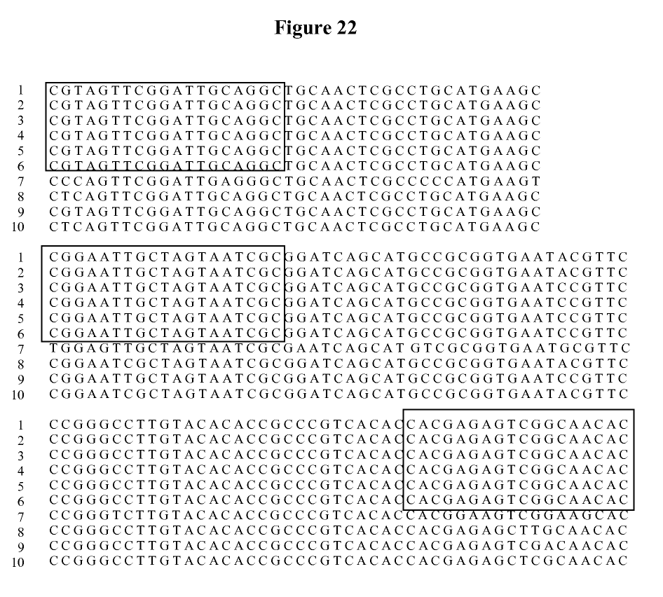

Development of CC16S primers and probe. Sequence data obtained from the Plant-Microbe Genomics Facility was compiled and entered into the NCBI BLAST network to check sequence integrity. Sequence data for each strain corroborated with respective sequence data in the GenBank. The 16S rDNA sequences from the three sequenced strains, as well as from A. acidoterrestris strain DSM 3923 (AB042058), A. cycloheptanicus strain DSM 4006 (AB042059), and A. acidocaldarius strain DSM 454 (AB059664) were used as positive controls in the alignment to determine a suitable priming region. B. thermoleovorans strain ATCC 43513 (M77488) and Clostridium elmenteitii isolate E2SE1-B (AJ271453) were used as negative controls in the alignment. In addition, closely related Geobacillus subterraneus strain K (AF276307) and Sulfobacillus disulfidooxidans SD-11 (U34974) were added to the alignment. Using the criteria described in the methodology, a forward and reverse primer and fluorogenic probe were derived, named CC16S-F, CC16S-R, and CC16S-Probe respectively. The sequences for the oligonucleotides are shown in Table V. This oligonucleotide set will amplify a 134 bp segment of the 16S rDNA. The alignment of the 134 bp priming region is shown in FIG. 22, with the selected primer and probe oligonucleotide sequences boxed around the Alicyclobacillus strains. These sequences were entered into the BLAST search network in order to discover identities with other unrelated organisms to ensure their specificity for Alicyclobacillus. Results show that the priming sequences are specific for 16S rDNA sequences of the three Alicyclobacillus species sequenced. In addition, the priming sequences also match the newly discovered species A. hesperidum, A. herbarius, A. acidiphilus, and A. sendaiensis. Also, it was found after alignment and BLAST searches that the priming region was highly similar to the members of the Geobacillus and Sulfobacillus genera, two closely related groups. Primers CC16S-F and CC16S-R were ordered from Sigma-Genosys (The Woodlands, Tex.), and the CC16S-Probe was ordered from Biosearch Technologies (Novato, Calif.). CC16S-Probe was labeled with the reporter dye Quasar 670 on the 5′ end, and quencher dye BHQ-2 on the 3′ end.
Real-time PCR specificity assay. Real-Time PCR is a new method has been developed to overcome the problems of standard PCR while increasing sensitivity and allowing for nearly instantaneous results. Real-time PCR adds an optical module and a fluorogenic probe to a standard PCR assay, while including computer-based data analysis software for real-time monitoring. Real-time PCR eliminates the need for post-amplification analysis and is not affected by non-specific amplification. The optical module attached to the thermal cycler detects a fluorescent signal that is emitted from the labeled probe at each cycle during the annealing stage. The amount of emission is recorded by computer software and plotted as an exponential curve, displaying the cycle at which a significant amount of amplification takes place.

The fluorescent reporter dye is held on the 5′ end of an oligonucleotide probe, with a quenching dye on the 3′ end to capture fluorescence not related to amplification. When the probe anneals within the primed region, the 5′ exonuclease activity of the polymerase in the reaction system cleaves the probe, inhibiting the quencher dye and increasing the emitted fluorescence from the 5′ reporter dye (21).

A real-time PCR assay was developed to test the specificity of the primers and probe for A. acidoterrestris, A. acidocaldarius, and A. cycloheptanicus. The assay also included E. coli DH-5α, L. lactis subsp. lactis, and P. putida to test for any unwanted cross-reactions with common foodborne microorganisms. In addition, Geobacillus stearothermophilus ATCC 10149 was included in the assay since it is a closely related thermophile of the Bacillus subfamilies. Assays were performed in triplicate, and results analyzed using the iCycler iQ Optical System Software. The results show that the reaction is specific for the three Alicyclobacillus while not reacting with E. coli DH-5α, L. lactis subsp. lactis, or P. putida. However, G. stearothermophilus had a positive reaction within the system.

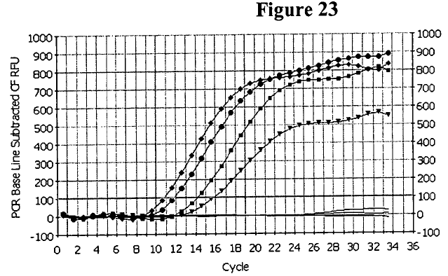

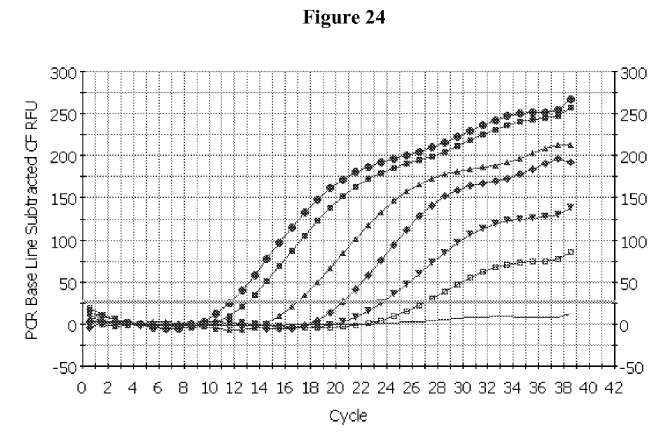

Real-time PCR sensitivity assay and limit of detection. After establishing system specificity, sensitivity of detection was determined. In order to accomplish this, tenfold serial dilutions in a 0.85% NaCl solution were made using A. acidoterrestris ATCC 49025 cultures. Real-time PCR assays were run in triplicate and results were analyzed using the iCycler iQ Optical System Software. A typical result is shown in FIG. 23. Quantification of the lowest detection level was performed through colony counting of plated dilutions used in the PCR. Colonies were counted on OSA plates and then averaged. The CFU/ml was calculated, and cell counts were determined for the lowest positive curve by multiplying the CFU/ml by the dilution factor of the curve. Data for cell counts and detection limits is presented in Table VI. In FIG. 24, the lowest accurate curve presented is from a 10⁻⁵ dilution, which is equivalent to 160 CFU/ml by plate count. Sensitivity tests were performed in triplicate, with the limit of detection ranging between 66 and 160 cells. The mean detection limit is 103 cells.

TABLE VI
A. acidoterrestris cell counts and corresponding detection
limits for sensitivity tests performed in
saline solution and orange juice.

| | | | Mean cell count | Minimum PCR | Mean PCR |
|---|---|---|---|---|---|
| | | Mean number | per replicate | detection level | detection level |
| Replicate | Media | of colonies^{a} | (CFU/ml) | per replicate^{c} | for trial set^{d} |
| 1 | Saline | 8 | 8.3 × 10^{6b} | 8.3 × 10¹ | Saline solution |
| 2 | Saline | 160 | 1.60 × 10⁷ | 1.60 × 10² | 1.03 × 10² |
| 3 | Saline | 66 | 6.6 × 10⁶ | 6.6 × 10¹ | |
| 1 | Orange Juice | 21 | 2.1 × 10⁷ | 2.1 × 10¹ | Orange juice |
| 2 | Orange Juice | 63 | 6.3 × 10⁷ | 6.3 × 10¹ | 5.36 × 10¹ |
| 3 | Orange Juice | 76 | 7.6 × 10⁷ | 7.6 × 10¹ | |

^{a}Diluted samples of A. acidoterrestris in respective media were plated on replicate plates of BBL Orange Serum Agar (Difco, Detroit), and colony counts and averages were obtained after 48 h at 47° C.
^{b}Calculation is estimated because no plates with between 20 and 200 colonies were available.
^{c}Minimum detection level is calculated by multiplying the mean cell count per replicate by the dilution level of lowest positive real-time PCR detection curve from the corresponding amplification run.
^{d}This is the calculated average detection limit for repeated real-time PCR trials in each type of media.

The detection limit of the Alicyclobacillus real-time PCR rapid screening system was also established in beverages using orange juice as a diluent. Serial dilutions were performed as previously described with juice in place of 0.85% NaCl. Juice samples were initially run in parallel with samples in 0.85% NaCl, and CT values and curve intensities were found to be comparable in both systems. Results for the assay in orange juice are shown in FIG. 25. Colony counting was performed on plated dilutions used in the PCR in order to determine cell counts at the minimum detection level. Data for cell counts and detection limits is presented in Table VI. In FIG. 25, the lowest accurate curve presented is from a 10⁻⁶ dilution, which is equivalent to 63 CFU/ml. Sensitivity tests were performed in triplicate, with the limit of detection ranging between 21 and 76 cells. The mean detection limit is 54 cells.

The efficiency of the system has also been tested in other beverages including apple juice, three sports drinks and Lemonade purchased from local grocery stores. These beverages were spiked with A. acidoterrestris cultures followed by cell collection, DNA extraction and real-time PCR detection. In all these cases, expected PCR amplification results were obtained indicating no particular inhibition by the ingredients from these tested beverages.

### Discussion

A specific and sensitive real-time PCR-based rapid detection system for Alicyclobacillus has been developed. In the past, PCR based assays have been used to detect microorganisms in different environments (16, 2, 17, 18, 19, 20, 28). More recently, the use of real-time PCR has been a favorable alternative to standard PCR based assays due to the increased speed and sensitivity of the results, the ability to quantify detection levels, and the elimination of post-amplification analysis (21). The present method was developed by targeting the 16s rDNA gene of Alicyclobacilli, using A. acidoterrestris, A. acidocaldarius, and A. cycloheptanicus as models for primer and probe development. However, the developed primers and probe could also be beneficial in detecting newly classified members of Alicyclobacillus, due to high sequence identity as shown by the BLAST data. This real-time PCR assay is an improvement over traditional culture methods of detection and PCR based detection systems. Culture methods can take between three and seven days for results to be available (12, 13). While accurate, the time frame is much too long for practical industry implementation. PCR assays provide much quicker results, but false positives can be easily detected (21), and gel electrophoresis analysis must be performed after amplification. Real-time PCR assays can be readily implemented in the industry because of the real-time results. Samples can be taken from the floor as they are produced and the presence of Alicyclobacilli can be detected within 3 hours.

In this study, the developed primers and probes were able to specifically detect A. acidoterrestris, A. acidocaldarius, and A. cycloheptanicus without cross-reaction with other common foodborne microorganisms. In addition, the system could also detect the presence of G. stearothermophilus.

### Example 2

A real-time PCR based rapid system was developed for detecting spoilage Alicyclobacillus spp. in foods. A common gene of Alicyclobacillus spp. encoding squalene-hopene cyclase, a key enzyme involved in hopanoid biosynthesis, was targeted for specific primers and probe development. Using the combination of the primers and probe, specific detection of the presence of representative strains from Alicyclobacillus spp. was achieved in the Taqman-based real-time PCR assay without cross-reacting with other food-borne bacteria. The presence of around 100 cells in collected samples can be detected within several hours.

Food spoilage causes significant financial loss to the industry. Every year, about 10% of our food supplies are lost due to spoilage and a significant portion of the problem is because of the presence of spoilage microbial agents, particularly molds, yeasts, and bacteria capable of surviving moderate heat- and acidic-treatments. Due to the limitation of applying extreme processing conditions, which can significantly alter the physiochemical properties and nutritional values of many food products, proper detection screening for the presence of microbial spoilage agents in food becomes a prior choice for quality control in the food industry. However, conventional industry practices for microbial detection from plate counting to biochemical analysis take anywhere from 48 hours to a couple of weeks. These methods are especially unsuitable for products with limited shelf life such as fruit juices. Novel detection approaches enabling rapid and specific detection of spoilage microorganisms within hours are preferred.

While the polymerase chain reaction (PCR) has been used extensively for years to rapidly amplify targeted DNA sequence regions, certain shortcomings limit its application in diagnostics and detection. For instance, PCR product analysis must be carried out after amplification, giving rise to an issue of post-amplification contamination and carry-over contamination (Heid et al., 1996). Most importantly, a high ratio of false positive results are often associated with PCR due to non-specific binding of the primers and the subsequent non-specific amplification of products. Recently a real-time PCR technology has emerged as a powerful diagnostic tool in both medical and agricultural fields.

Using real-time PCR, a fluorescent dye such as SYBR green can be incorporated into the reaction mixture and the fluorescent signals, generated from fluorescent dye binding to double stranded DNA products, can be detected directly by the optical module coupled with the thermocycler. The signals are processed by computer data analysis software for almost real-time calculation and on screen plotting. A new dimension of real-time PCR called Taqman assay further introduced a third oligonucleotide probe, labeled with 5′ fluorescent reporter dye and 3′ quenching dye, for signal detection (Livak et al., 1995; Basseler et al., 1995). In the Taqman system, the quenching dye on the 3′ end captures the fluorescence from the 5′ reporter dye so the intact probe itself does not produce strong signal. During amplification when the probe hybridized to complementary sequence within the amplified products, the 5′→3′ exonuclease activity of the polymerase in the reaction system cleaves the probe, minimized the quenching effect and the emitted fluorescent signal from the 5′ reporter dye can be detected by the optical module. An advantage of applying the Taqman system is that a double complementing sequence selection mechanism by both the primers and the probe is involved, therefore the false positive rate of the detection can be significantly cut down. So far, various Taqman real-time PCR-based detection approaches have been reported. However, reports on its application in the real food system are still limited. The greatest challenges are (i) effective extraction of DNA and RNA from a system where microorganisms are mixed with the food matrix including bulk proteins, carbohydrates and fatty acids, (ii) selection of primer-and-probe sets that are specific for the target microorganisms and do not interaction with background microflora and food ingredients, and (iii) minimizing the influence of food ingredients and other chemical compounds in the food matrix on the action of enzymes involved in DNA extraction and amplification.

Our objective was to demonstrate the feasibility of the real-time PCR based detection technology for food industry applications. It is our understanding that due to the complication of various food systems, detection procedures likely need to be optimized for individual food commodities. In this study, we investigated the practicability of using the Taqman-based real-time PCR approach in detecting target microorganisms in juice products. Here we report the effectiveness of the Taqman-based detection system in rapid, specific and sensitive detection of spoilage A. acidocaldarius and A. acidoterrestris in juice products, using a primer-and-probe set specific for the shc gene encoding squalene-hopene cyclase.

### Materials and Methods

### Bacterial Strains and Growth Conditions.

The bacterial strains used in the study and their growth conditions were listed in Table VI. ATCC 573 medium consists of 1.3 g (NH₄)₂SO₄, 0.37 g KH₂PO₄, 0.25 g MgSO₄.7H₂O, 0.07 g CaCl₂.2H₂O, 1.0 g glucose, 1.0 g yeast extract, and 1.0 L distilled H₂O, pH 4.0. BAM-SM ATCC 1656 medium consists of 0.25 g CaCl₂.2H₂O, 0.5 g MgSO₄.7H₂O, 0.2 g (NH₄)₂SO₄ 3.0 g KH₂PO₄, 6.0 g yeast extract, 5.0 g glucose, 1.0 mL trace elements (0.66 g CaCl₂.2H₂O, 0.18 g ZnSO₄.7H₂O, 0.16 g CuSO₄.5H₂O, 0.15 g MnSO₄.4H₂O, 0.18 g CoCl₂.6H₂O, 0.10 g H₃BO₃, 0.30 g Na₂MoO₄.2H₂O, 1.0 L distilled H₂O), and 1.0 L distilled H₂O. Geobacillus stearothermophilus ATCC 10149 was grown in Nutrient broth (Difco). Stock cultures of all strains were stored in their respective media plus 40% glycerol and kept at −80° C. All inoculations used were 2% concentrations made from frozen cultures.

TABLE VII
Bacteria cultures used in the study.

| | Medium and | |
|---|---|---|
| Strains | Growth Condition | Resource |
| A. acidocaldarius ATCC43030 | #573 broth^{a} at 48° C. | ATCC |
| A. acidoterrestris ATCC49025 | #1655 broth^{a} at 48° C. | ATCC |
| A. cycloheptanicus ATCC49029 | #1656 broth^{a} at 48° C. | ATCC |
| Bacillus subtilis | Nutrient broth^{b}, 40° C. | |
| Geobacillus? | | |
| E. coli DH5α | LB broth, Miller^{c} at 37° C. | |
| Pseudomonus putidis? | LB broth, Miller at 37° C. | |
| Listeria monocytogenes V7 | Tryptic soy broth^{d} at 37° C. | |
| Lactococcus lactis 2301 | M17 broth^{e} at 37° C. | |

^{a}All numbered broth for Alicyclobacillus spp. are ATCC media.
^{b}From Becton Dickison & Co., Sparks, MD.
^{c}From Fisher Chem., Fais Lawn, NJ.
^{d}From Becton Dickison and Company, Sparks, MD.
^{e}From Becton Dickison and Company, Sparks, MD.

DNA extraction, gene cloning and DNA sequencing. For DNA extraction, cells were collected from 1 ml of bacterial culture by micro-centrifugation 7.6K rpm for 10 min. The cell pellet was treated with 20 mg/ml of lysozyme (Sigma Chemical CO. St Louis, Mo. 63178, USA) in buffer for 45 min at 37° C. Genomic DNA was extracted using the DNeasy® Tissue Kit (QIAGEN GmbH, D-40734 Hilden, Germany) and eluted into 100 μl of elution buffer following the instructions from the manufacturer.

The shc gene fragment from each strain was obtained by conventional PCR amplification using degenerate primers derived from conserved amino acid sequences and the genomic DNA from each strain as template. The reaction mixture includes 1×PCR buffer, 3 mM MgCl₂, 4 mM dNTP (Invitrogen, Carlsbad, Calif.), 1 μM primer pairs, 1 μl of genomic DNA template and ddH₂O in a total final volume of 50 μl. PCR was performed one cycle at 95° C. for 3 min, followed by 30 cycles at 95° C. for 30s, 50° C. for 30s and 72° C. for 1 min, with a final extension at 72° C. for 7 min using I-cycler (Bio-Rad, Hercules, Calif.). PCR products were purified using the QIAquick PCR purification kit (Qiagen, Valencia, Calif.) following manufacturer's instruction. Purified PCR products were cloned into pCR 2.1 vectors and transformed into One Shot competent Escherichia coli cells using the TA Cloning kit (Invitrogen, Carlsbad, Calif.). Recombinant plasmids were recovered using QIAGEN miniprep (QIAGEN GmbH, D-40734 Hilden, Germany). DNA sequences were determined using the ABI PRISM® 3700 DNA Analyzer (Applied Biosystems, Foster City, Calif.) at the Plant Genome Sequence Facility, The Ohio State University.

Real-time Tagman PCR conditions For real-time PCR, the reaction was conducted in thin-wall microcentrifuge tubes including 1×iQ™ Supermix (Bio-Rad, Hercules, Calif.), 0.5 μM of primer pair, 0.3 μM of probe, 10 μl of genomic DNA extraction and ddH₂O in a final volume of 50 μl. PCR was performed one cycle at 95° C. for 3 min followed by 40 cycles of 95° C. for 30s, 55° C. for 1 min using 1-cycler (Bio-Rad, Hercules, Calif.).
DNA sequence analysis. The DNASTAR (DNASTAR, Madison, Wis.) software package was used in DNA and protein sequence alignment and homology search. DNA oligonucleotide primer and probe sequences were also compared with sequences from the GenBank sequence database using BlastSearch.

Specificity and sensitivity analyses Assays were conducted to test the specificity of the detection system against spoilage Alicyclobacillus spp. and other common food-borne microorganisms. Genomic DNA was extracted from broth cultures of A. acidoterrestris and A. acidocaldarius, grown for 48 h at 48° C. (absorbance at OD₆₀₀ around 0.5-0.7), using the previously discussed DNA extraction protocol. Genomic DNAs extracted from 1 ml of overnight culture of Escherichia coli DH-5α, Lactococcus lactis subsp. lactis C2, Geobacillus stearothermophilus ATCC 10149 and Pseudomonas putida 49L/51 were also used in the specificity study. Ten micro liters out of the 100 micro liter of elution was used as template and the real-time PCR amplification was carried out using conditions described above but using 32 instead of 40 cycles of amplification.

The sensitivity tests of the real-time PCR assay for detection of Alicyclobacillus in bacterial culture media were performed using tenfold serial dilutions from 10⁰ to 10⁻⁸ of A. acidoterrestris in a 10 ml solution of 0.85% NaCl. The initial cultures were obtained by grown for 18 h at 48° C. using 2% inoculation from the frozen stock, with the absorbance reading at OD₆₀₀ range between 0.38 and 0.42. After serial dilution, cells from 1 ml of each sample were collected by centrifugation at 7600 rpm for 10 minutes for DNA extraction. Ten microliter out of the 100 microliter of elution was used as template and the real-time PCR amplification was carried out as described above.

Sensitivity tests in juice products were also performed in the same manner but the serial dilutions were carried in apple juice instead of saline.

In both sensitivity analyses, a range of dilutions between 10⁻⁴ and 10⁻⁵ were plated on acidified PDA agar (Difco, Detroit) for colony counting to compare with the results by Taqman real-time PCR. Plates were incubated at 48° C. for 48 h.

### Results

### 1. THE PRIMER-AND-PROBE SET USED IN THE REAL-TIME PCR TAQMAN ASSAY

Hopanoids are membrane components involved in maintaining membrane fluidity and stability (4) of Alicyclobacillus spp. in extreme environmental conditions. We have targeted the shc gene encoding squalene-hopene cyclase, a key enzyme in hopanoid biosynthesis, for PCR primer-and-probe development.

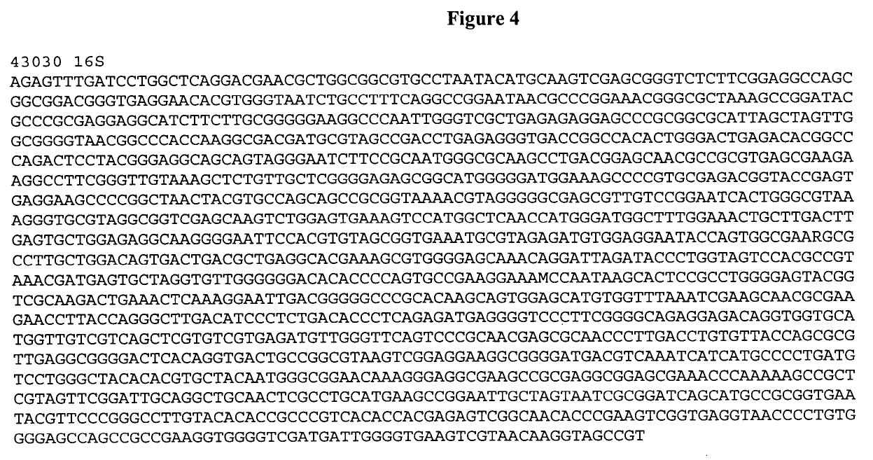

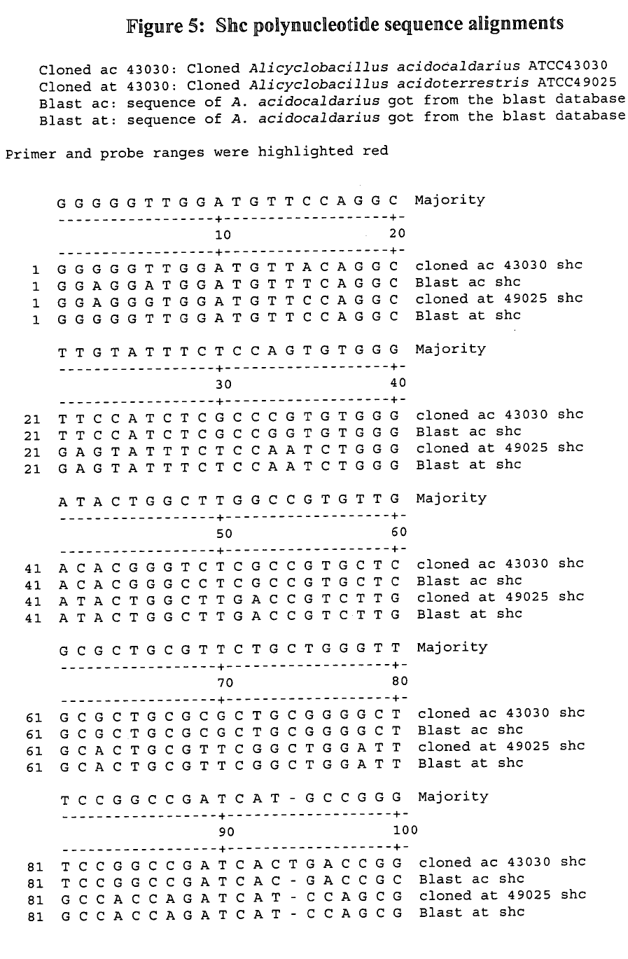

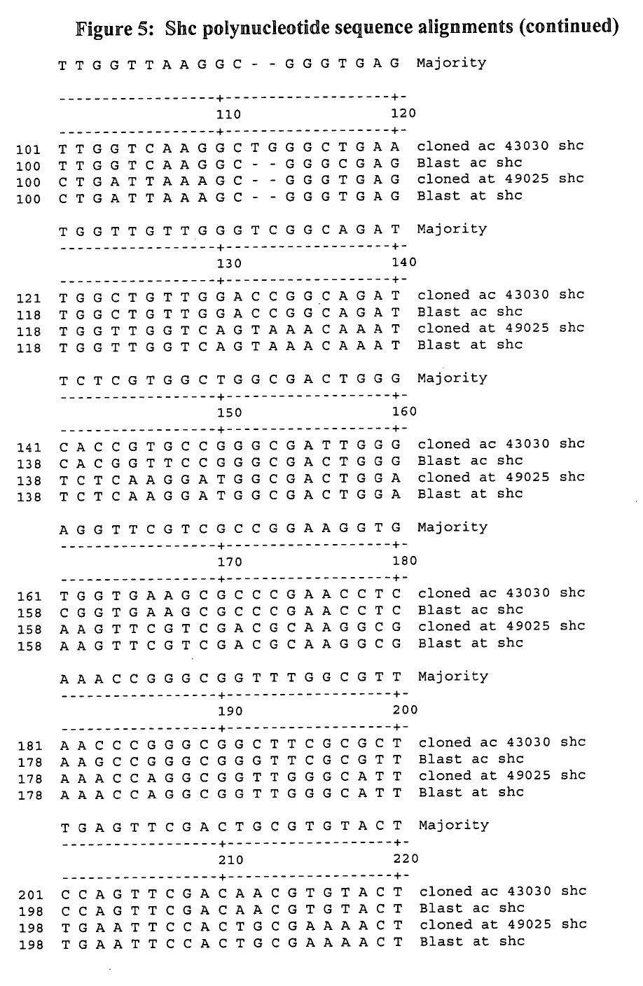

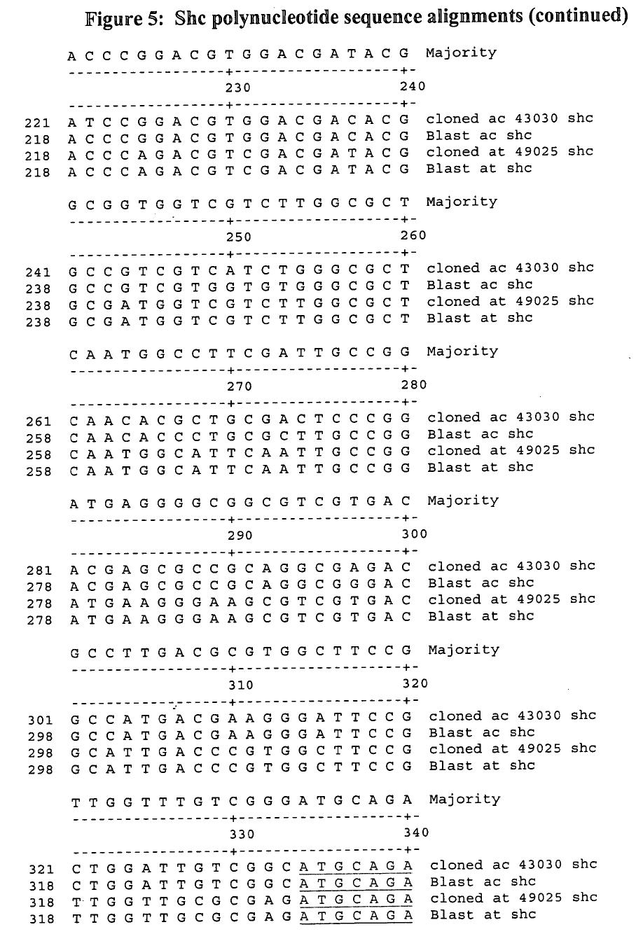

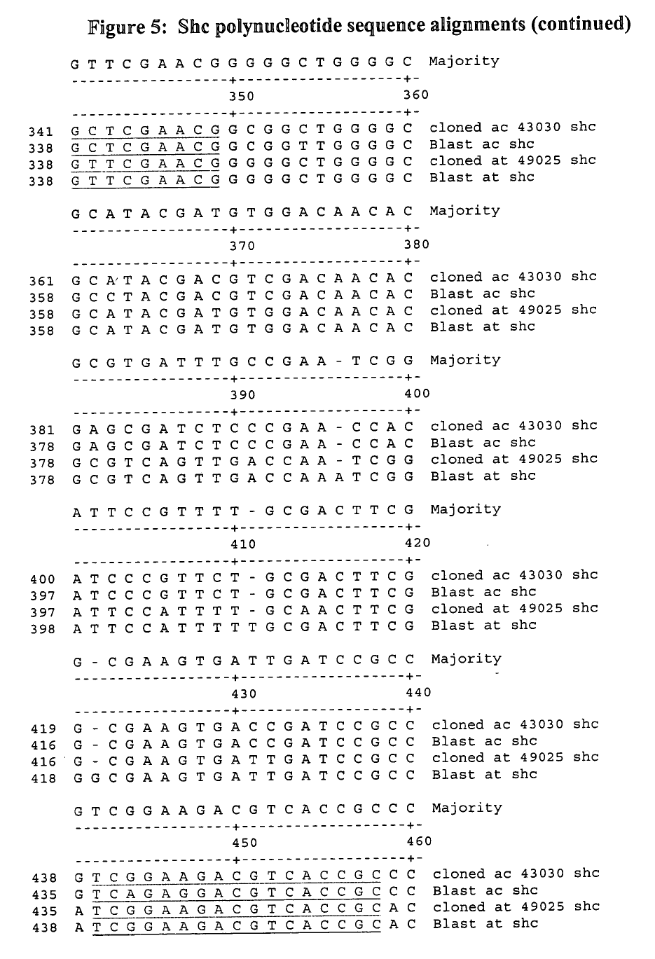

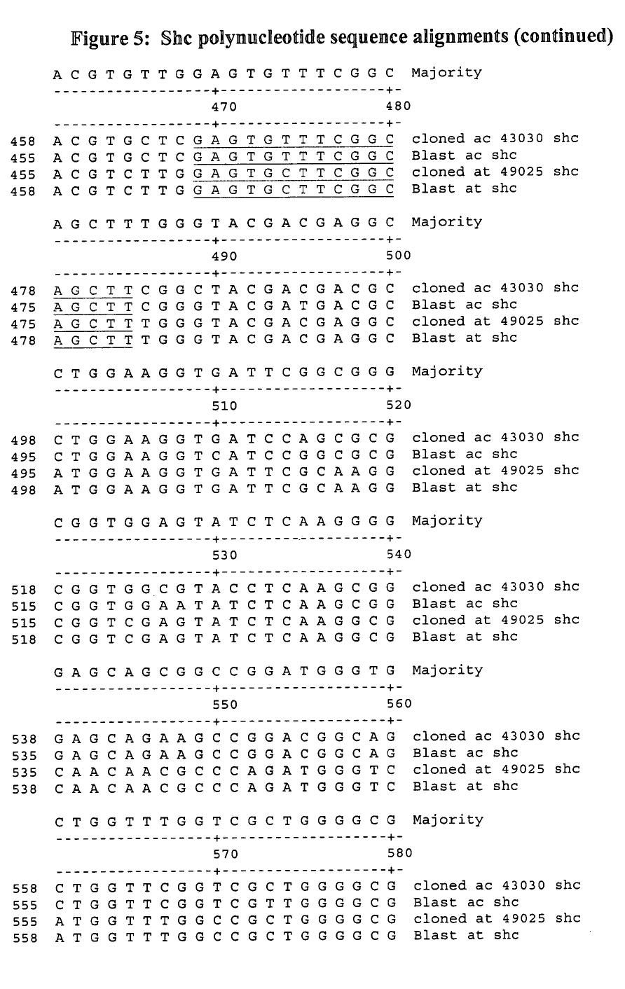

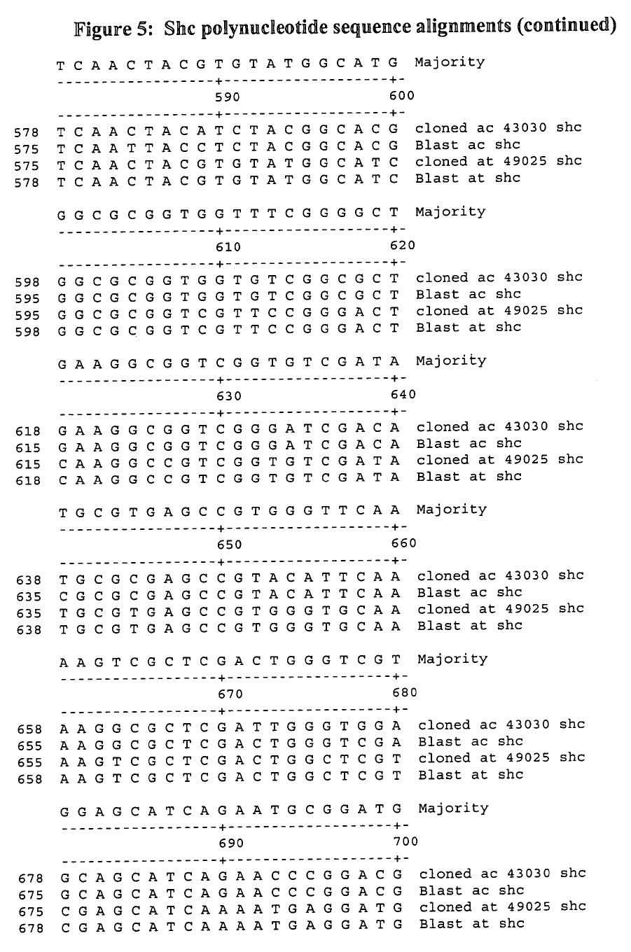

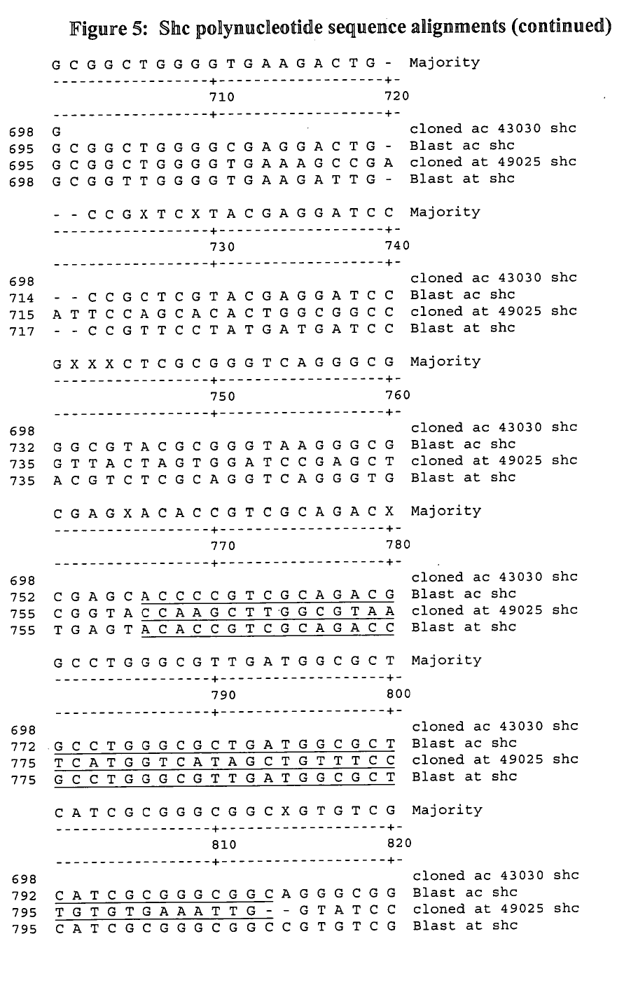

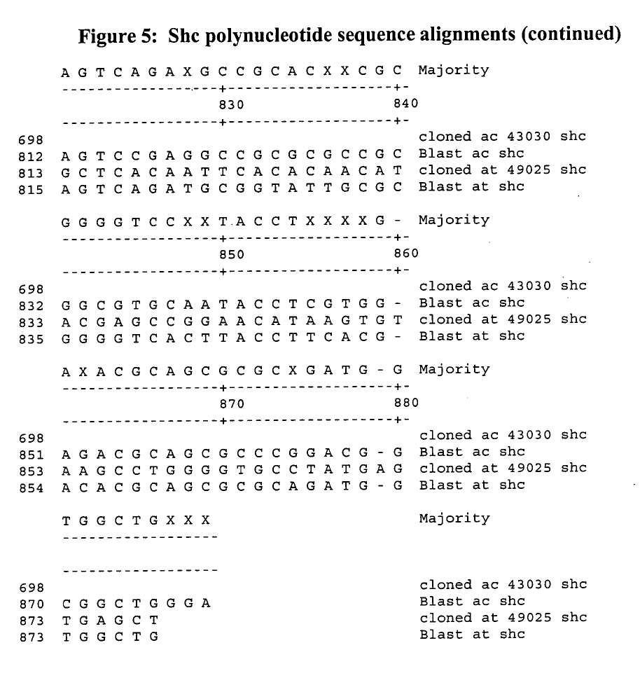

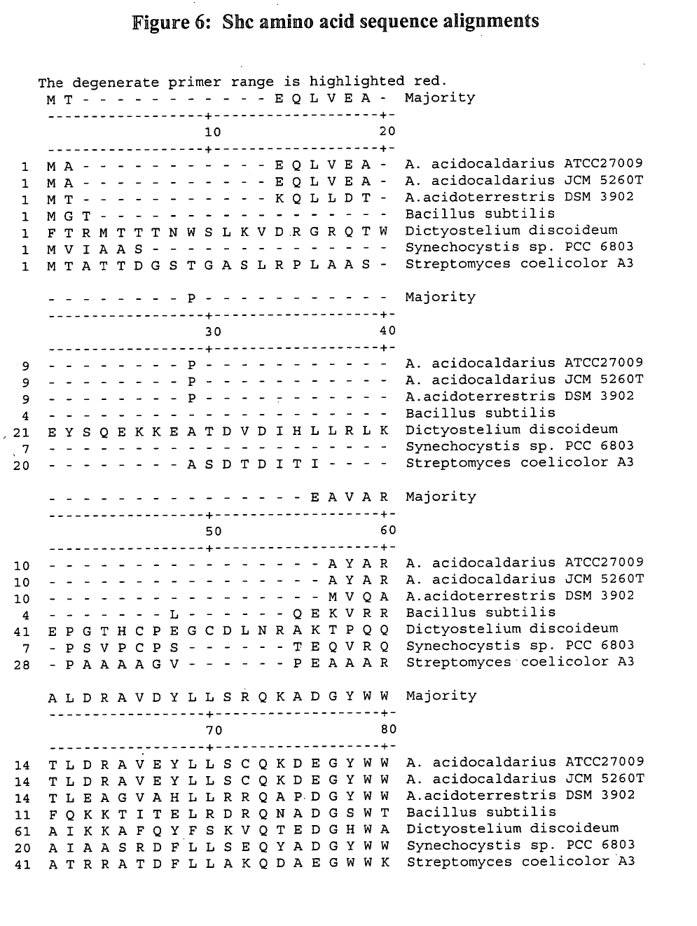

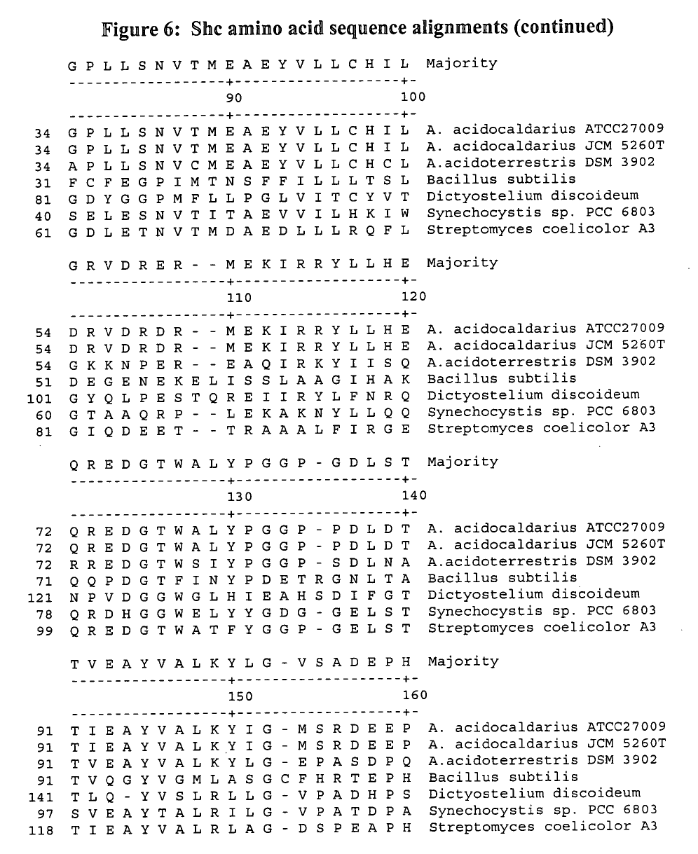

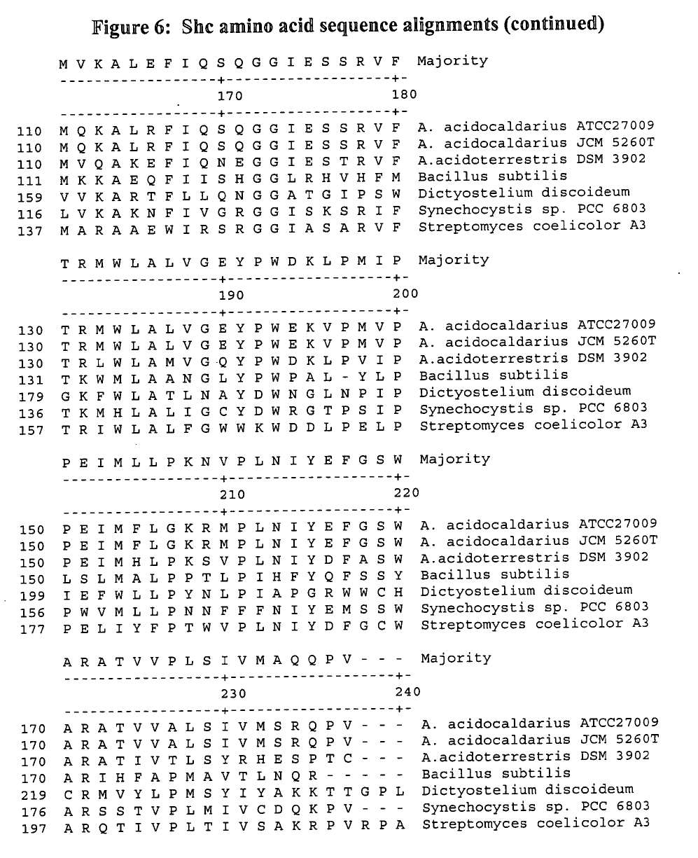

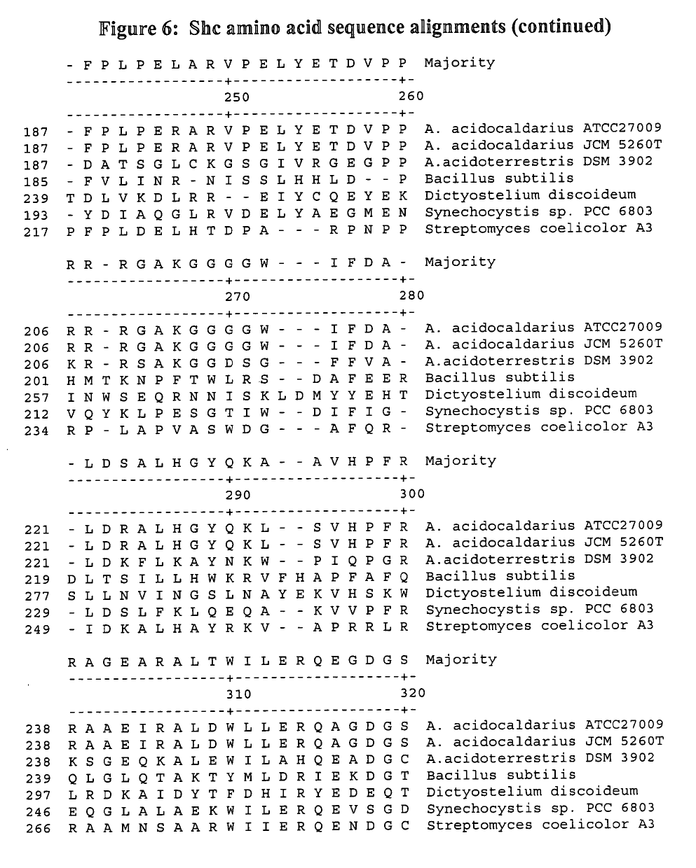

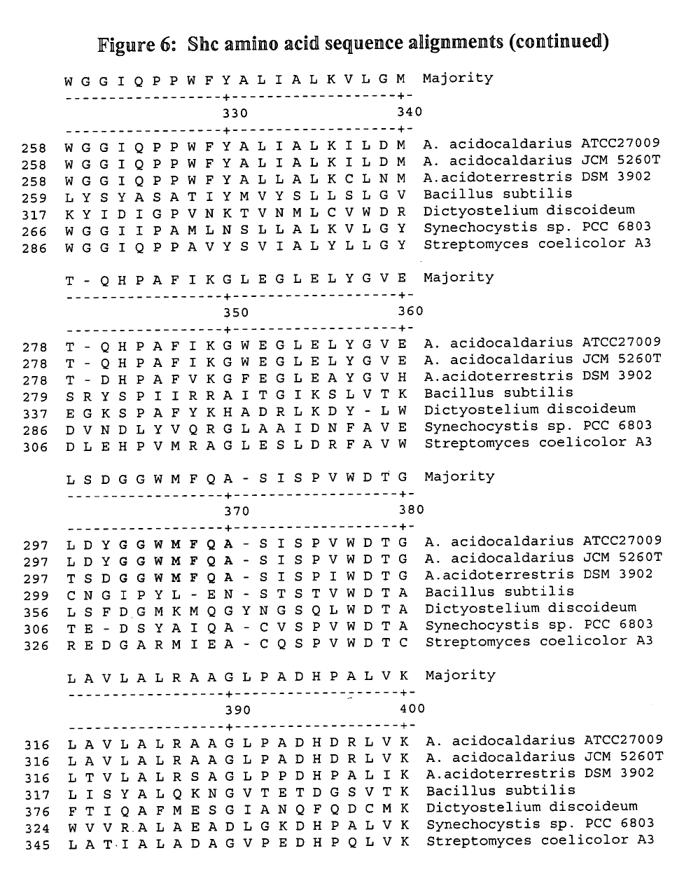

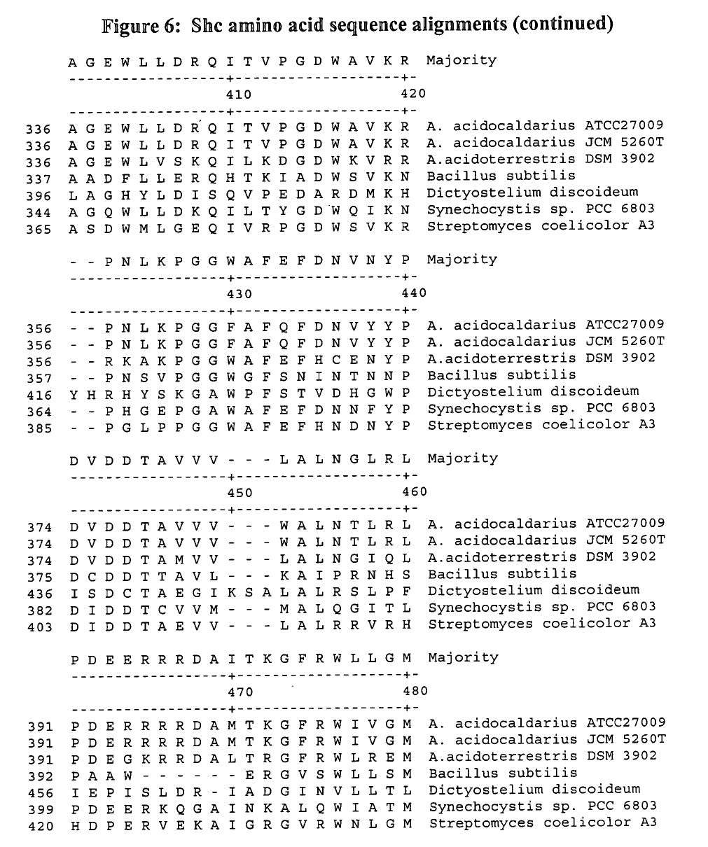

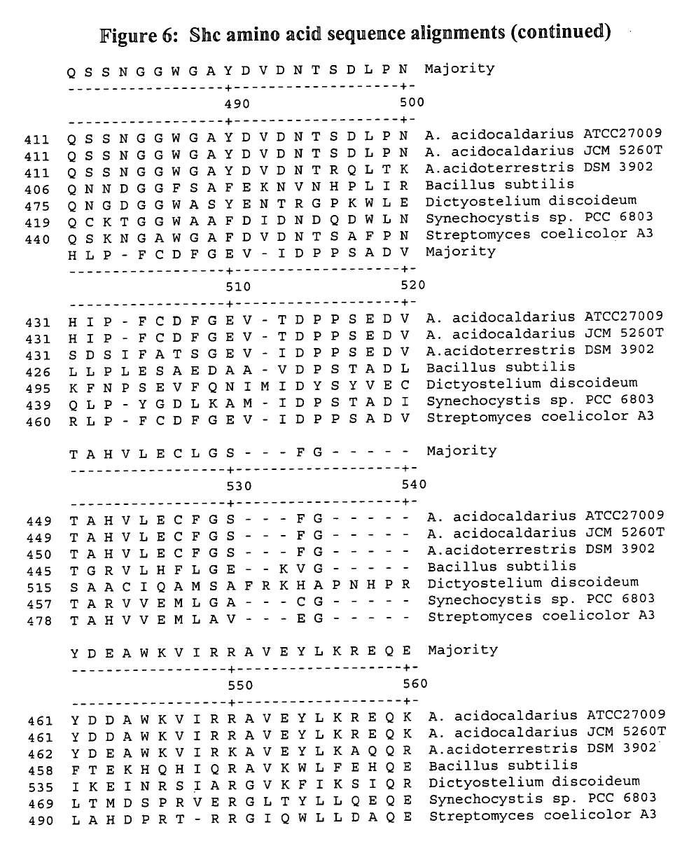

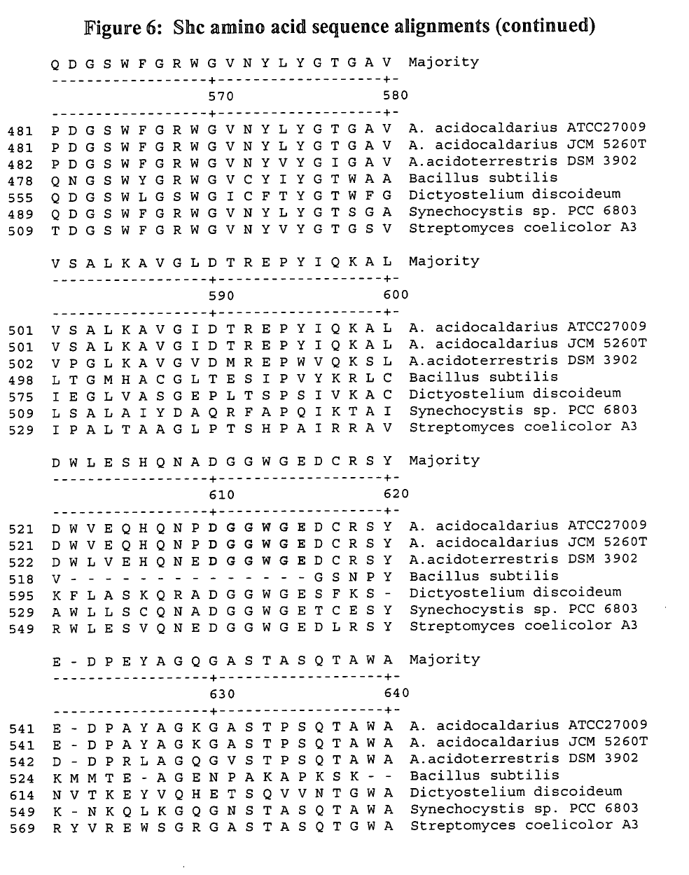

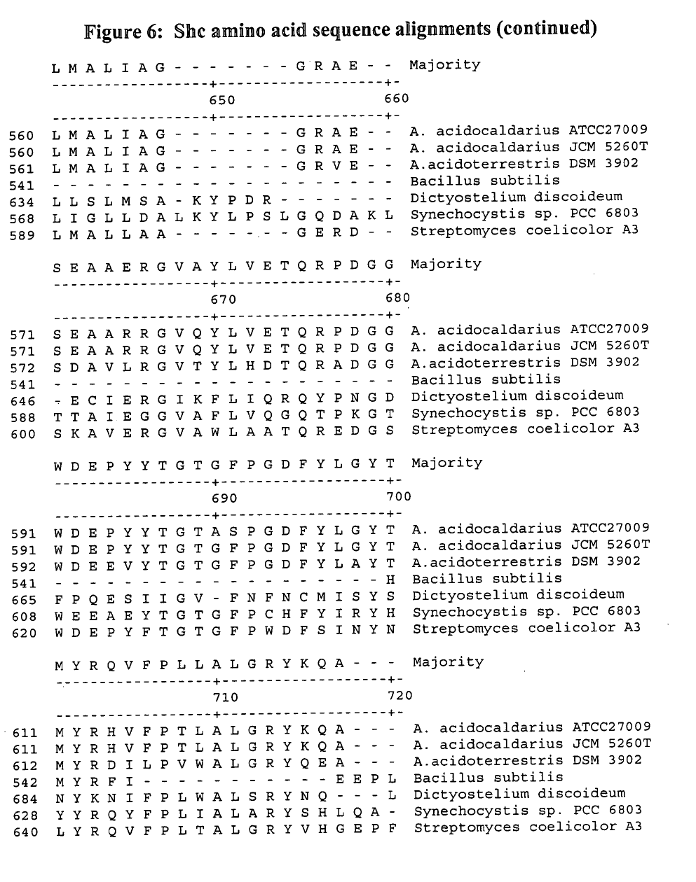

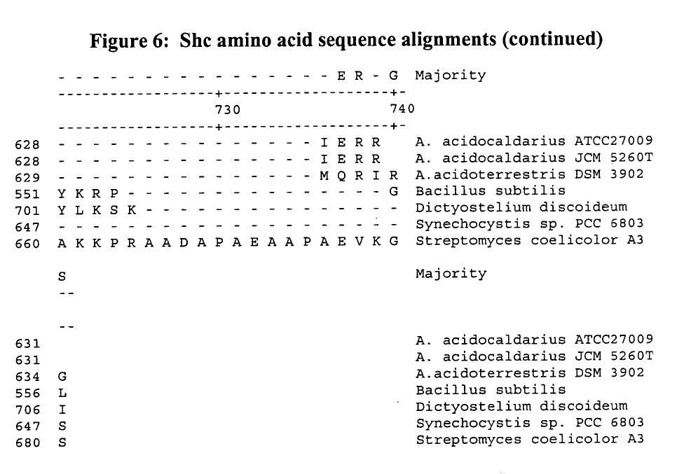

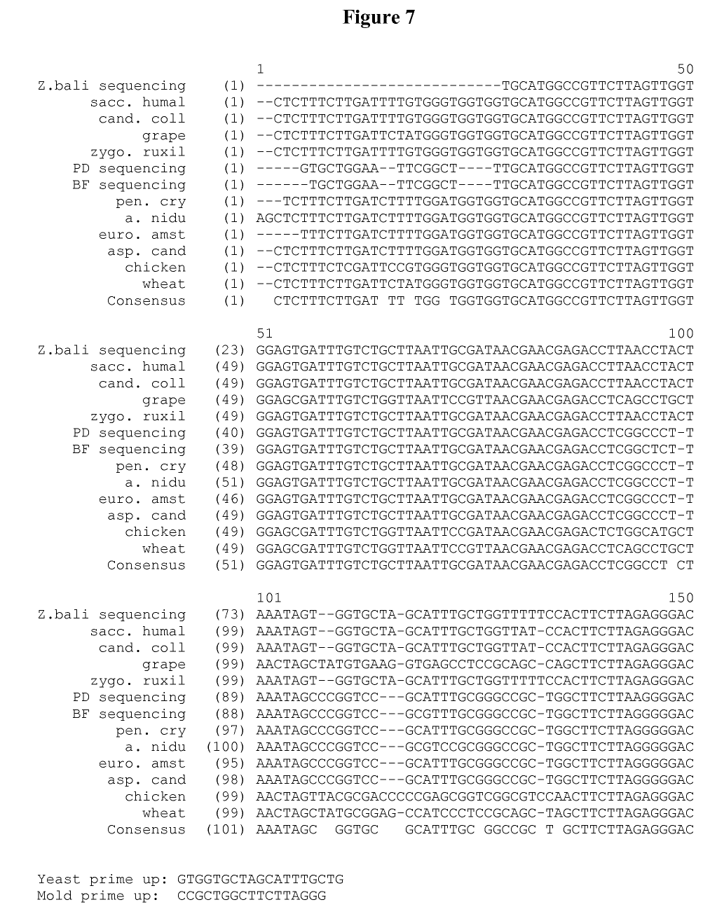

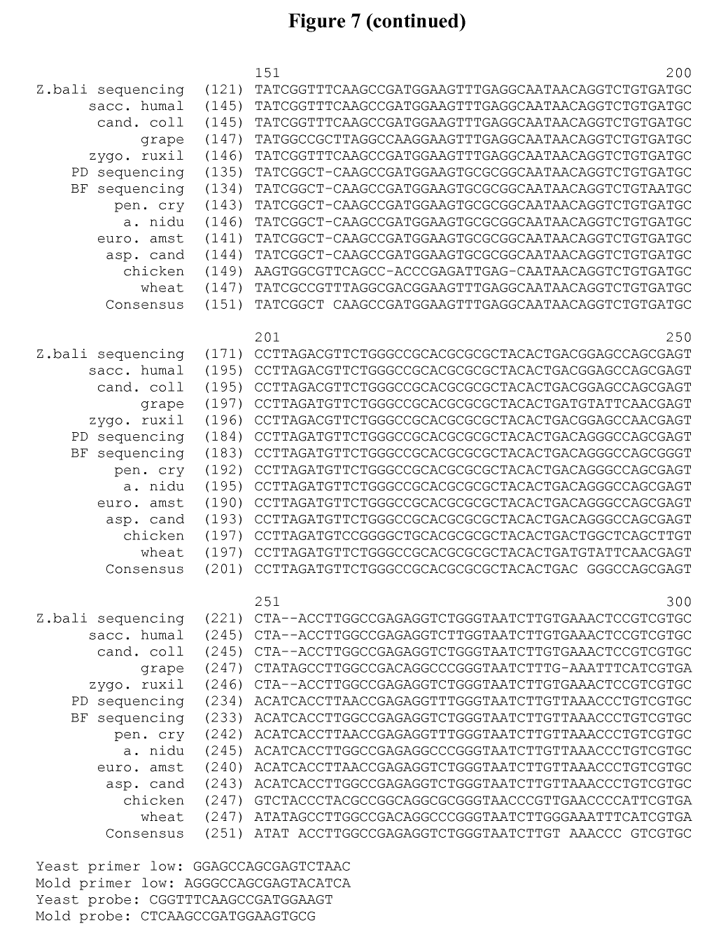

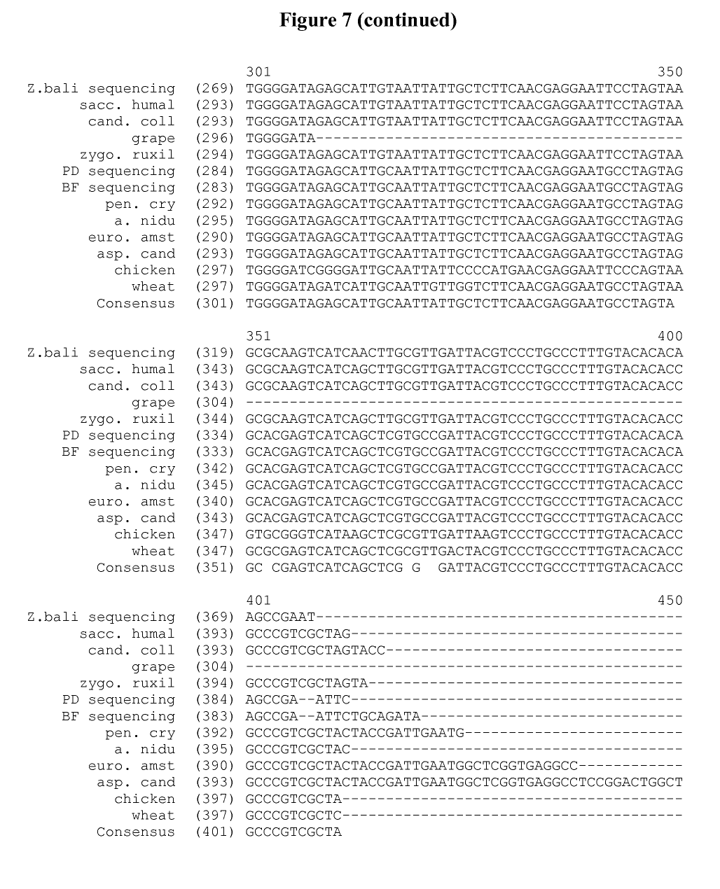

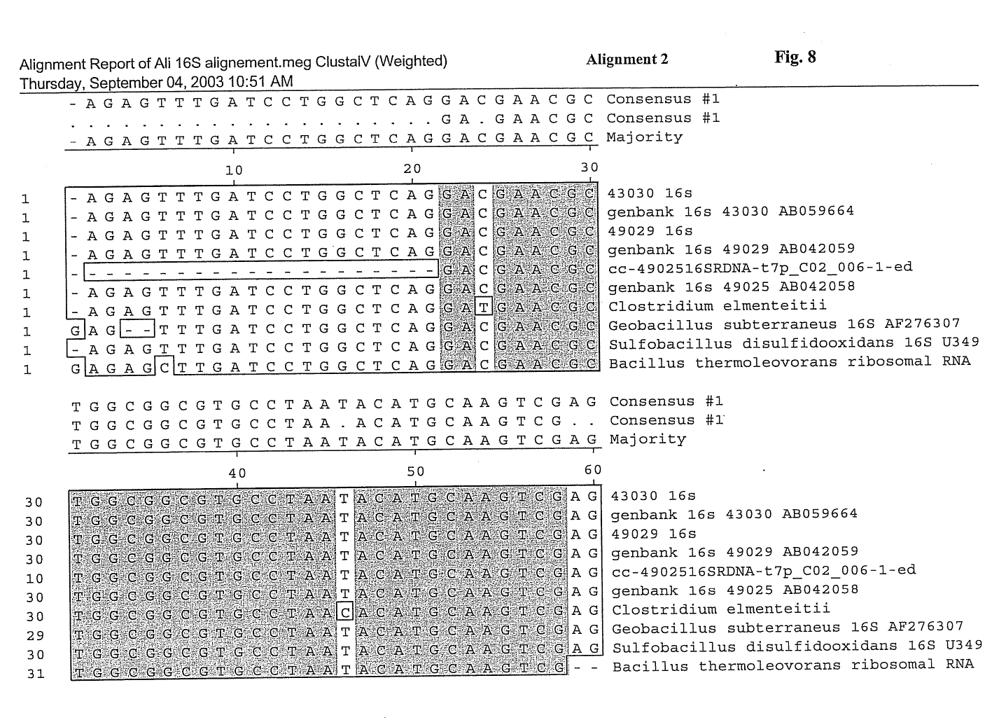

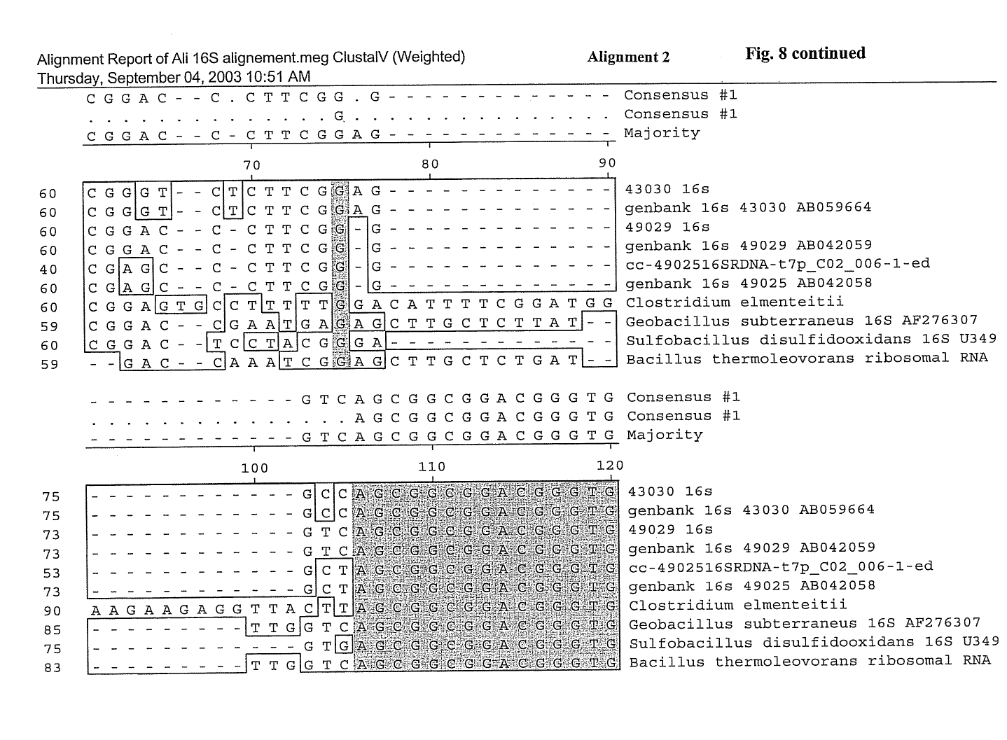

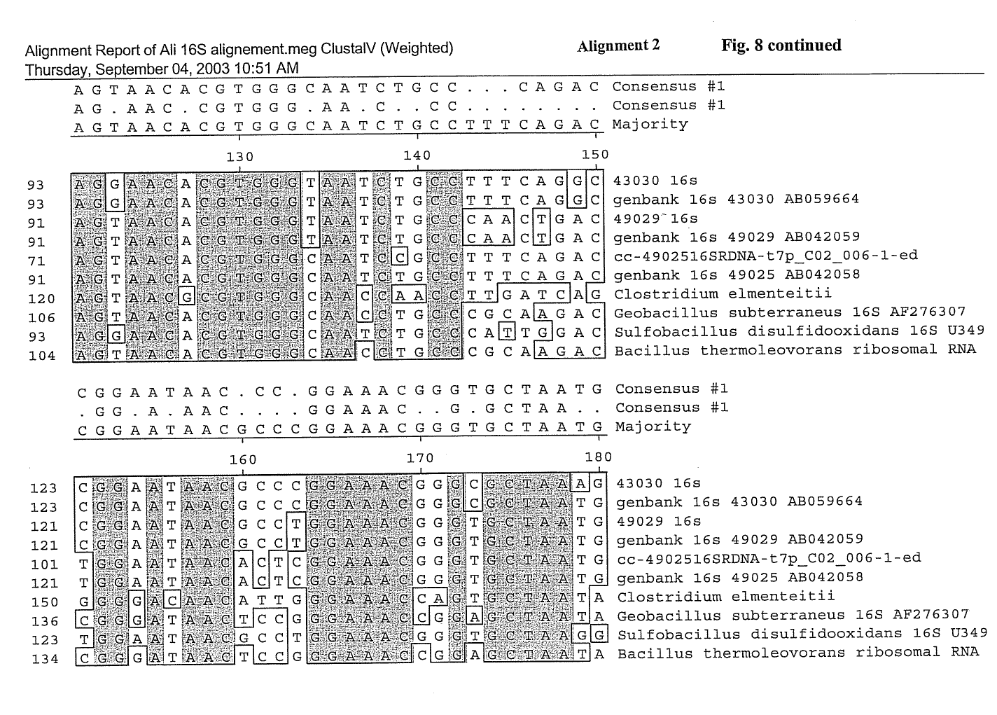

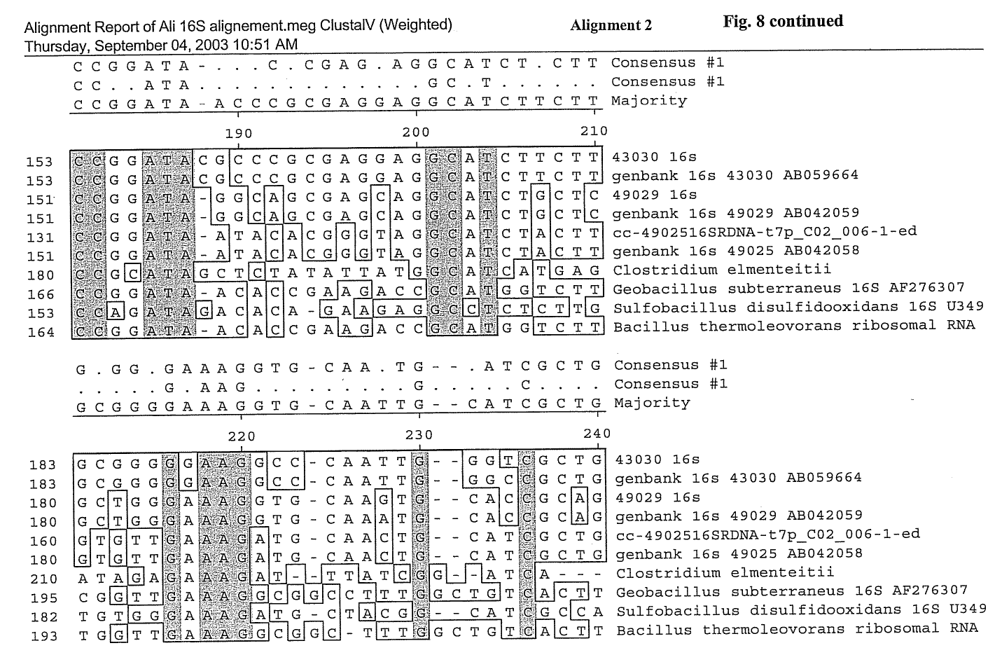

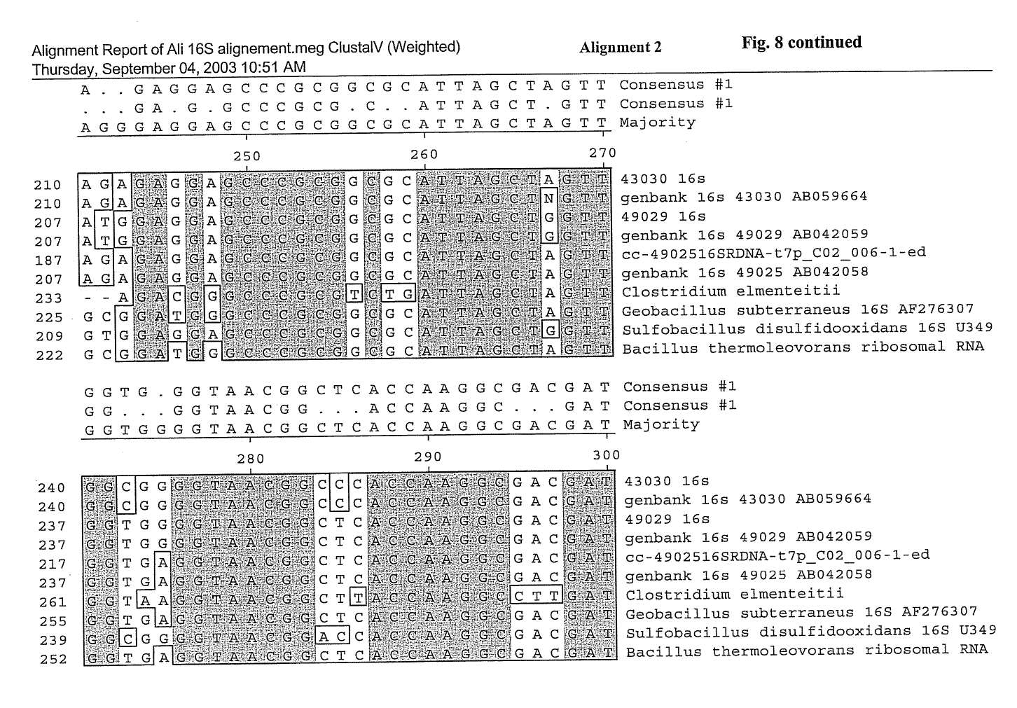

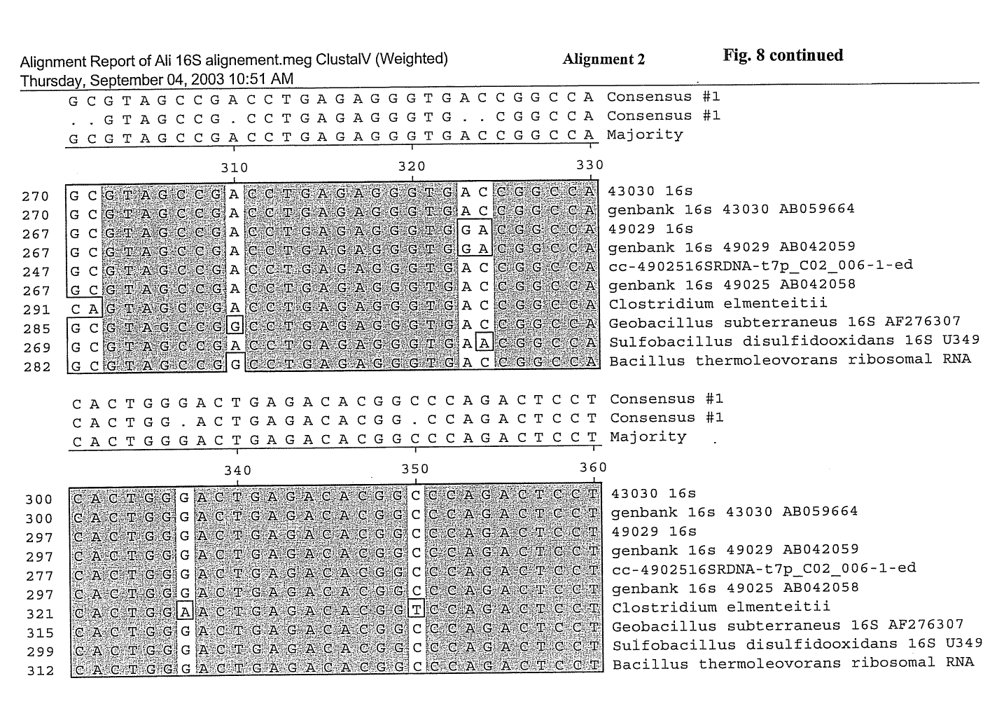

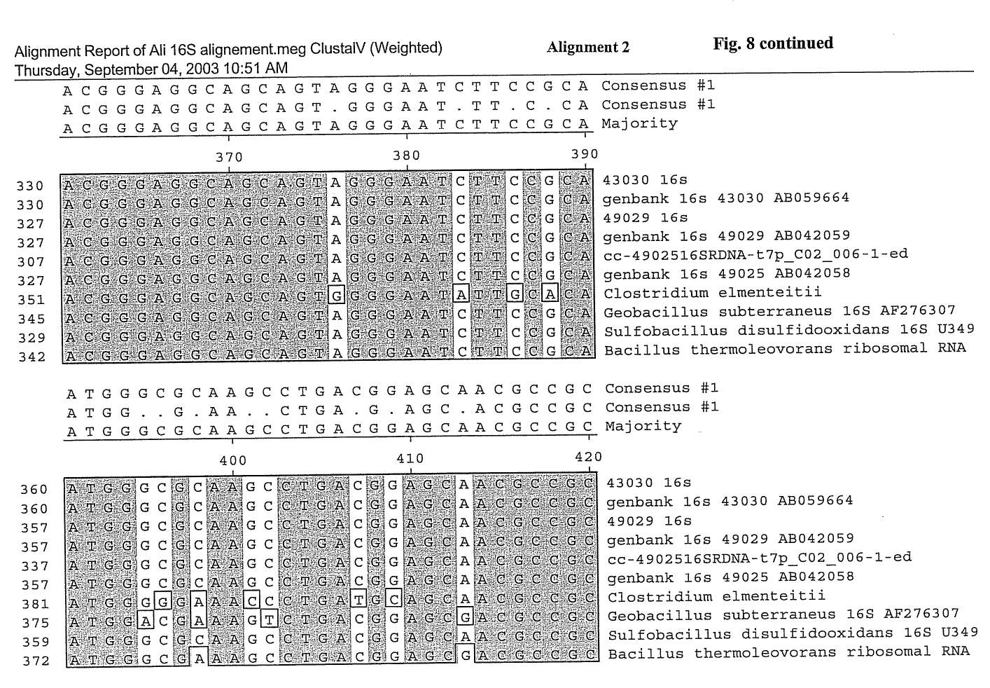

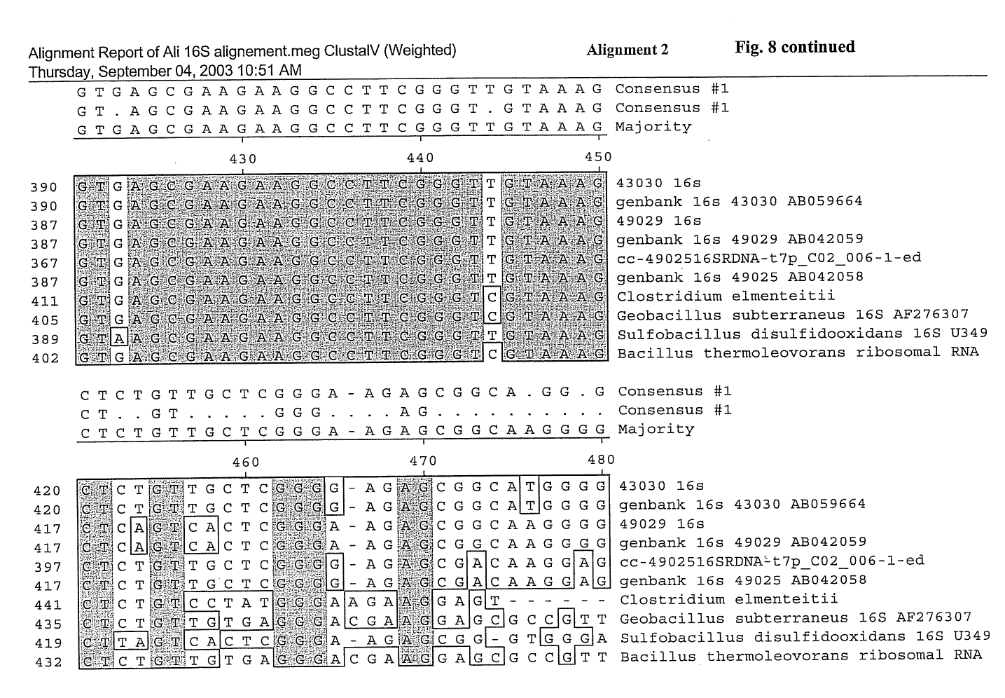

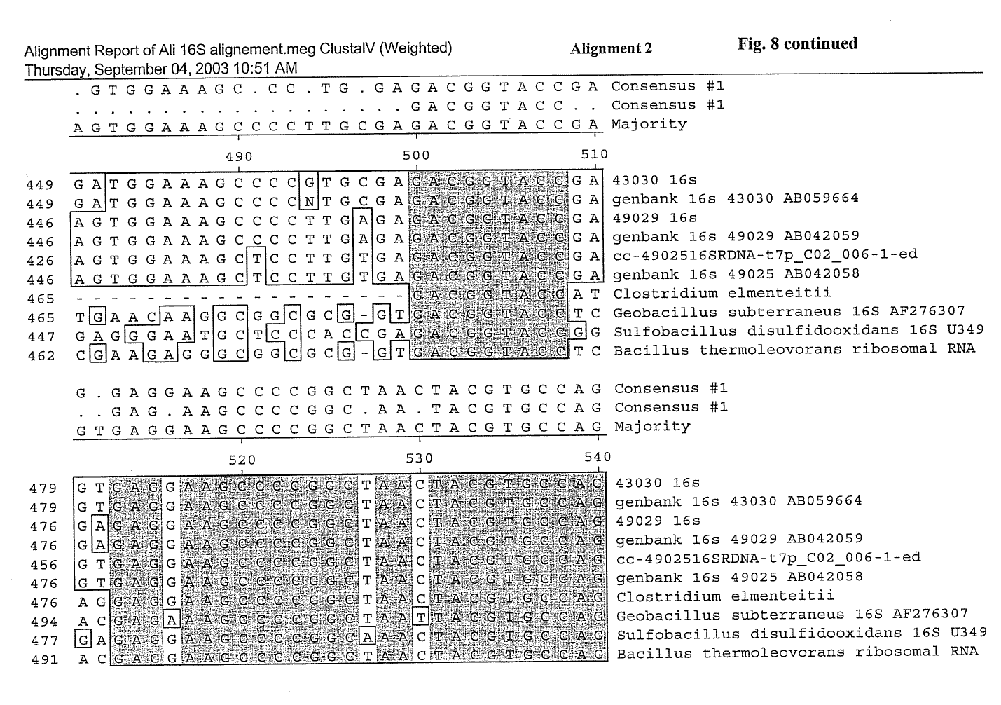

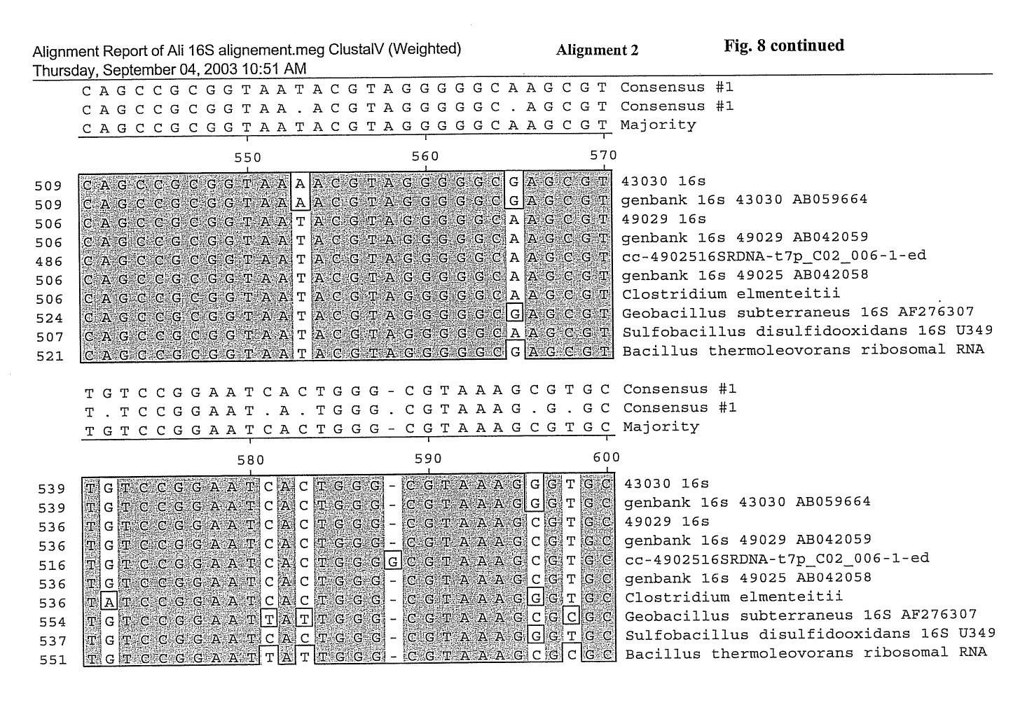

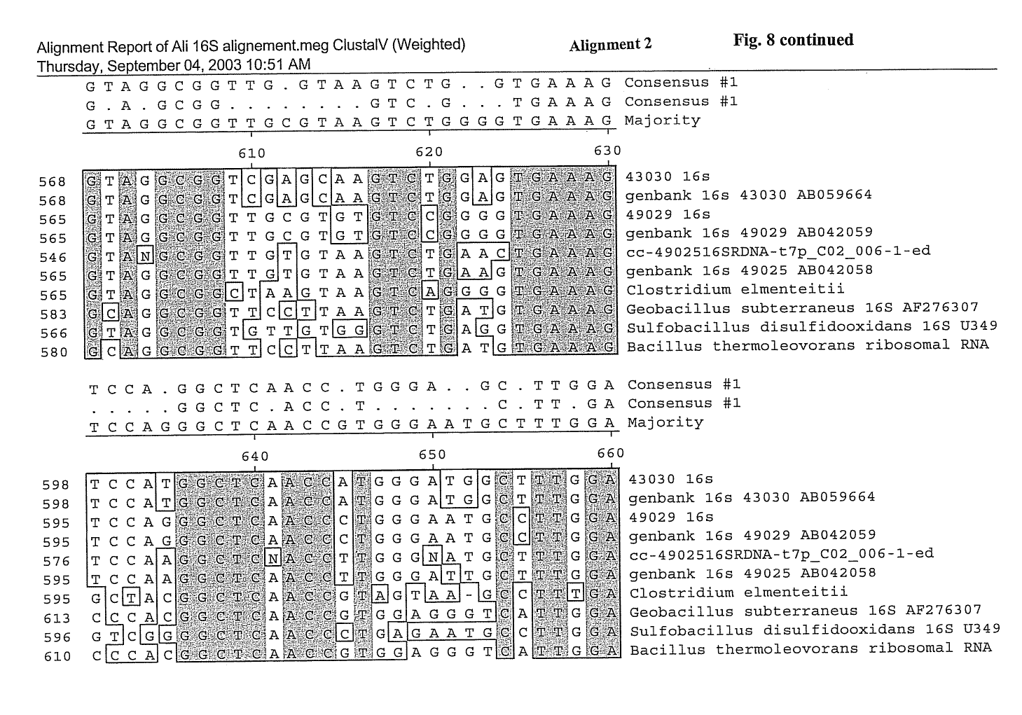

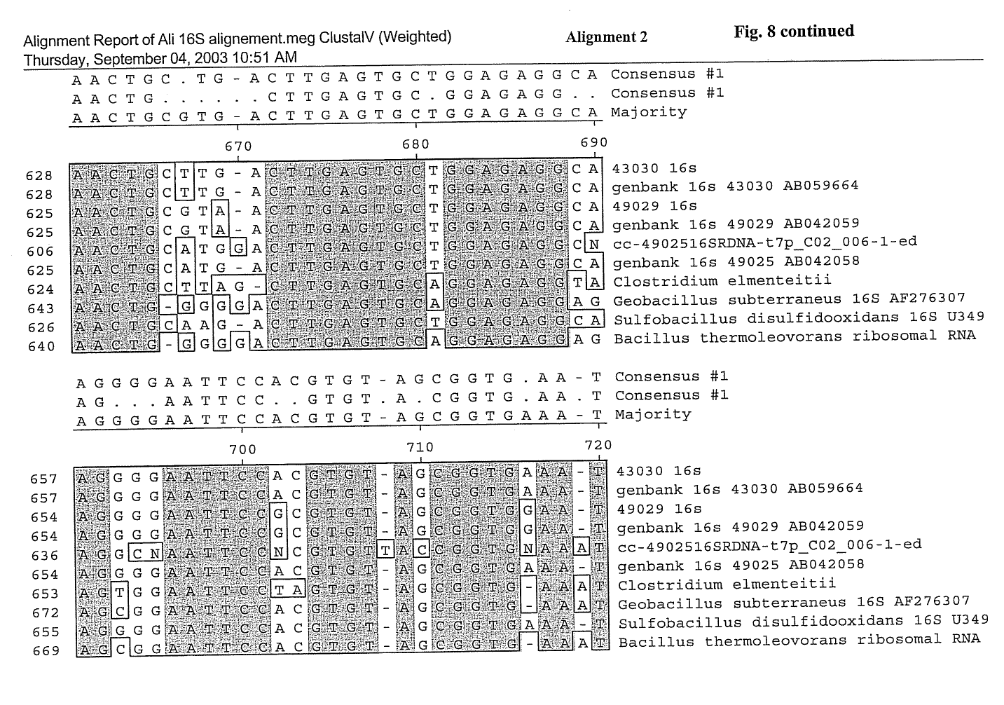

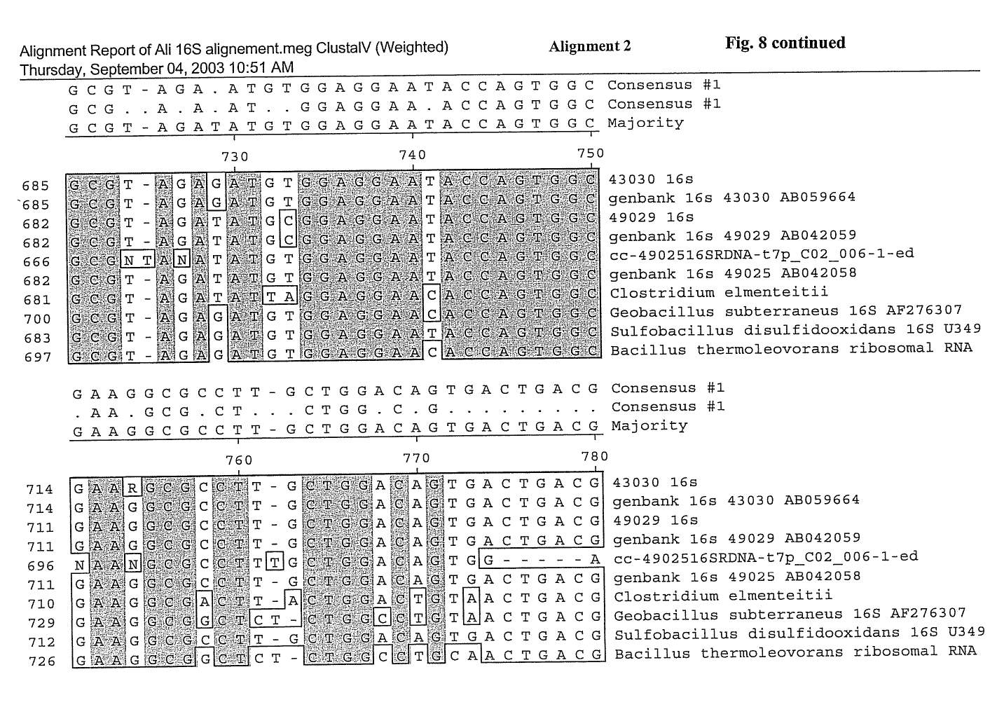

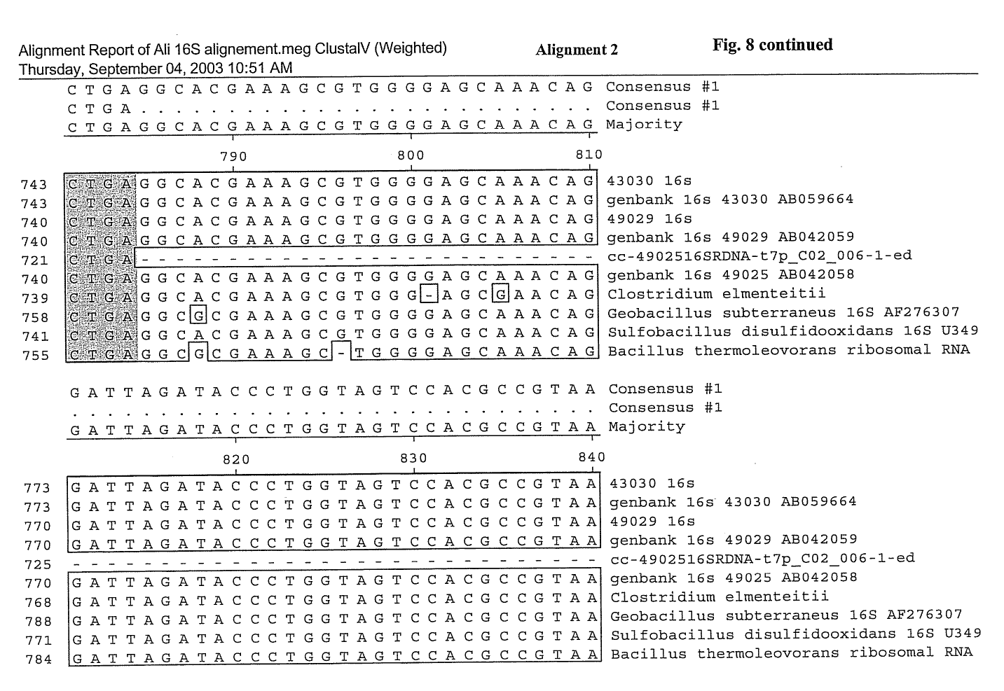

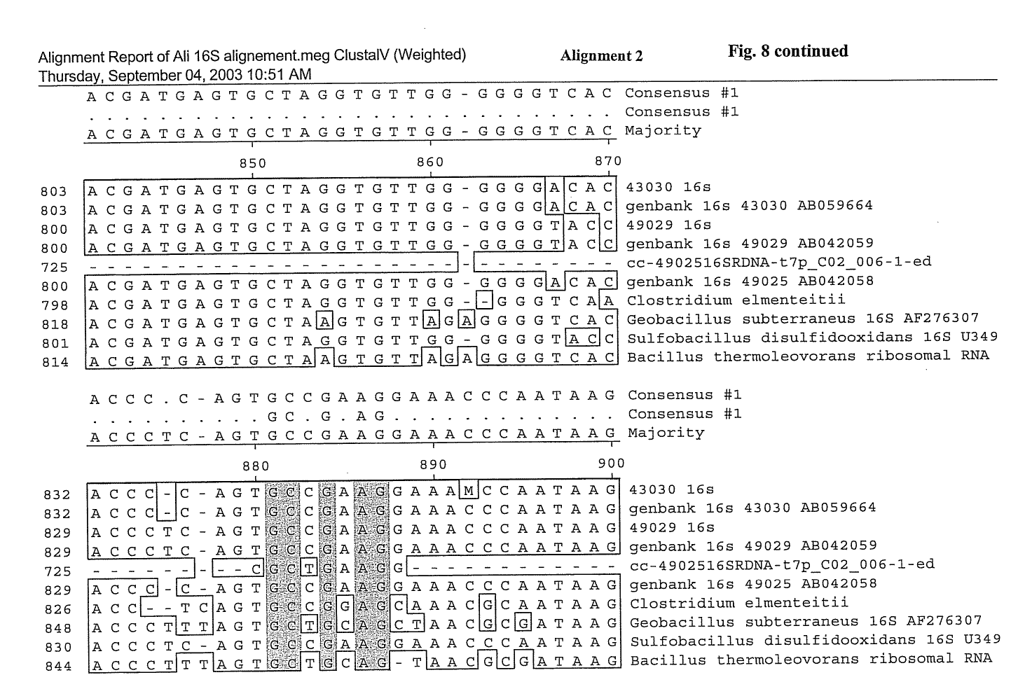

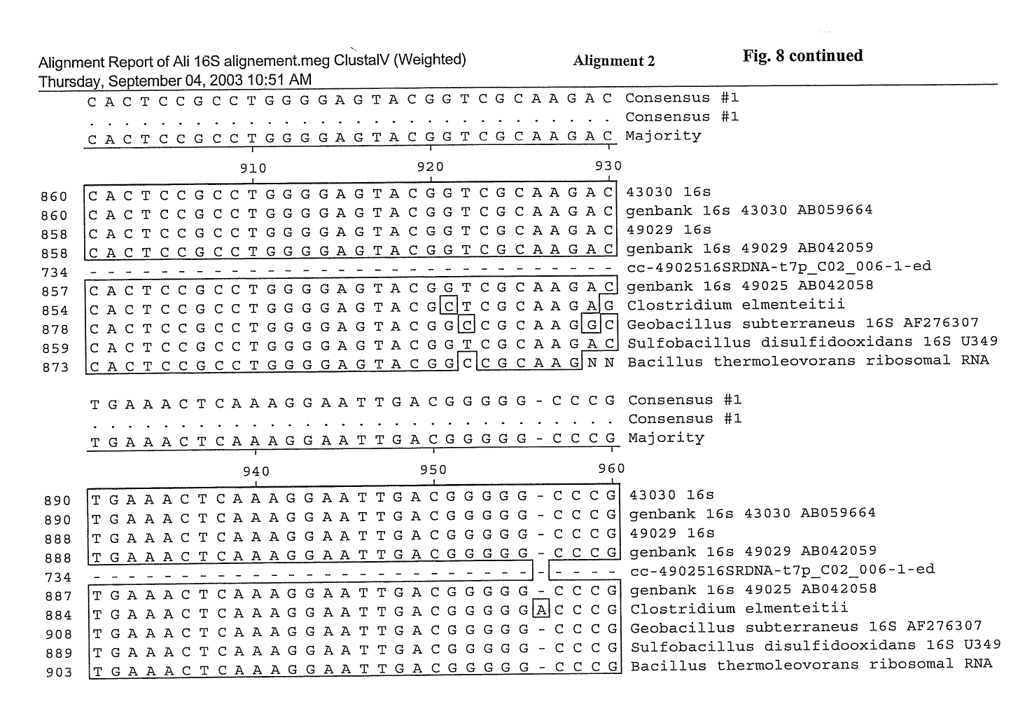

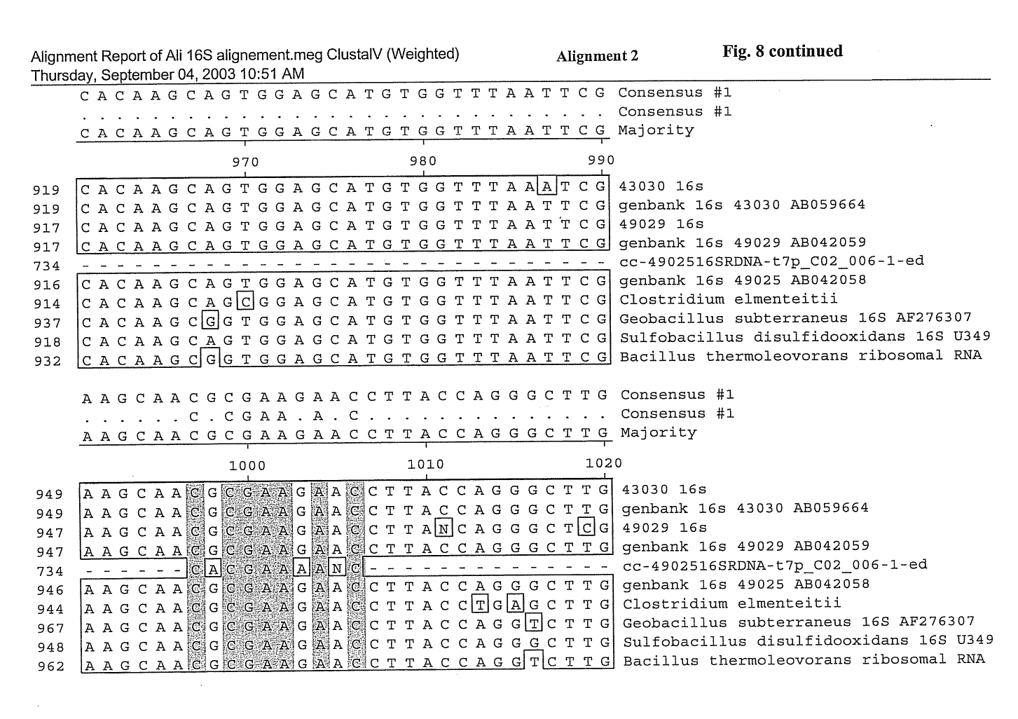

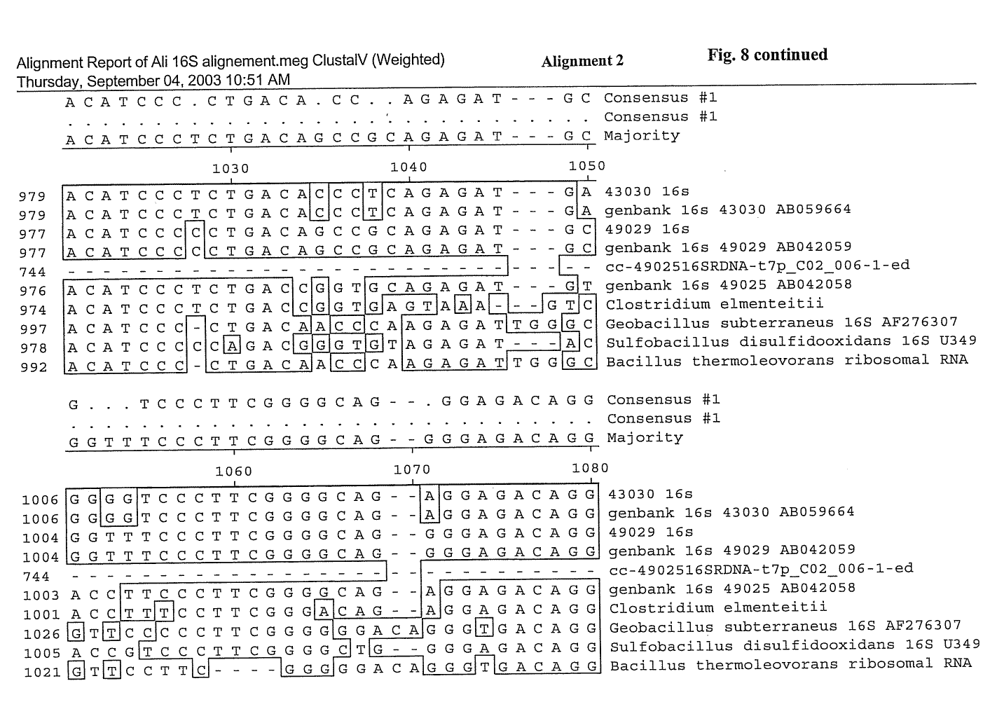

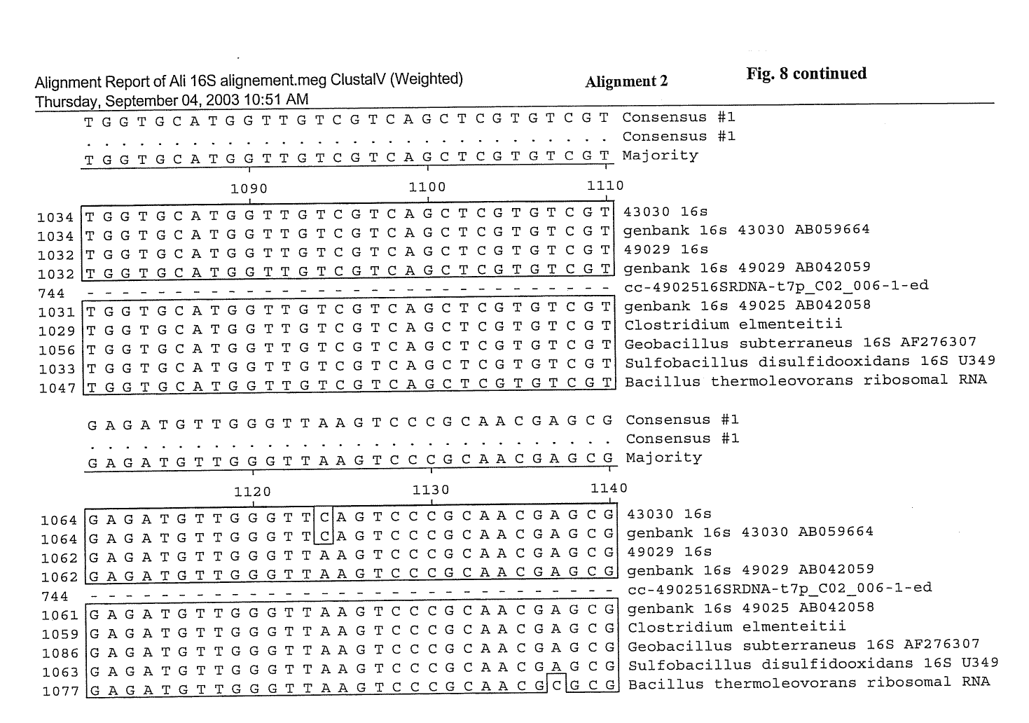

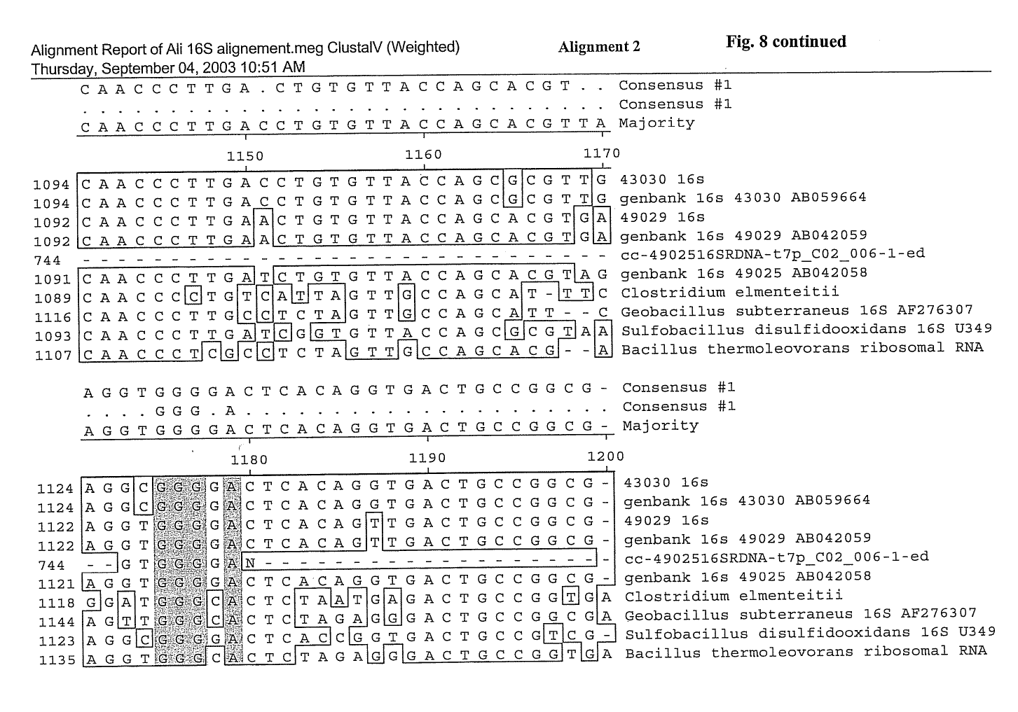

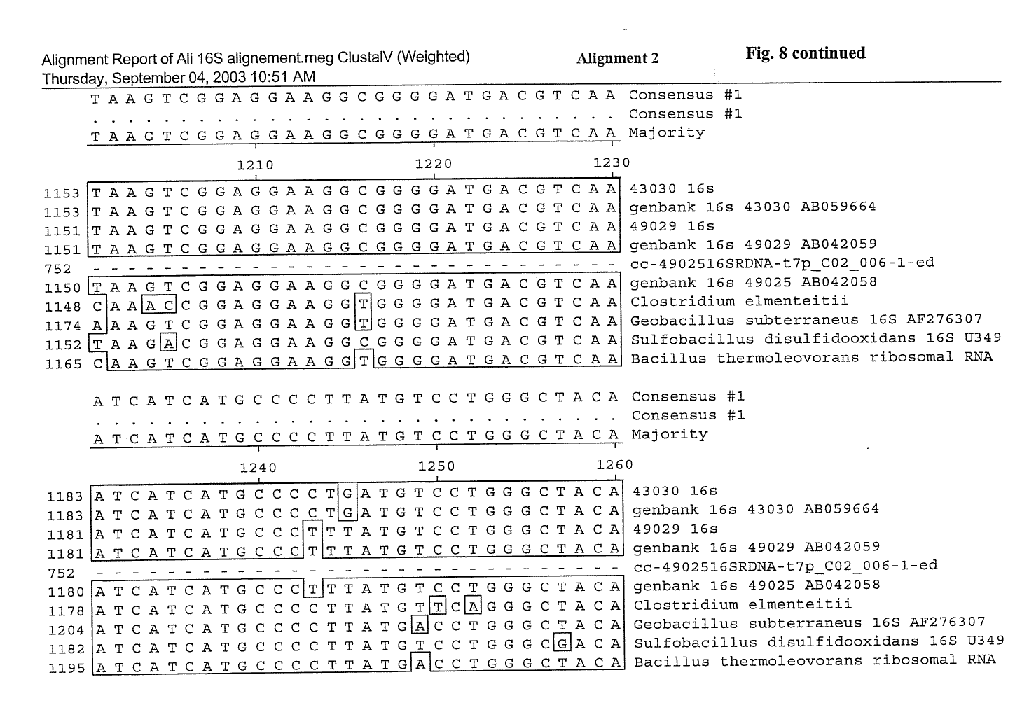

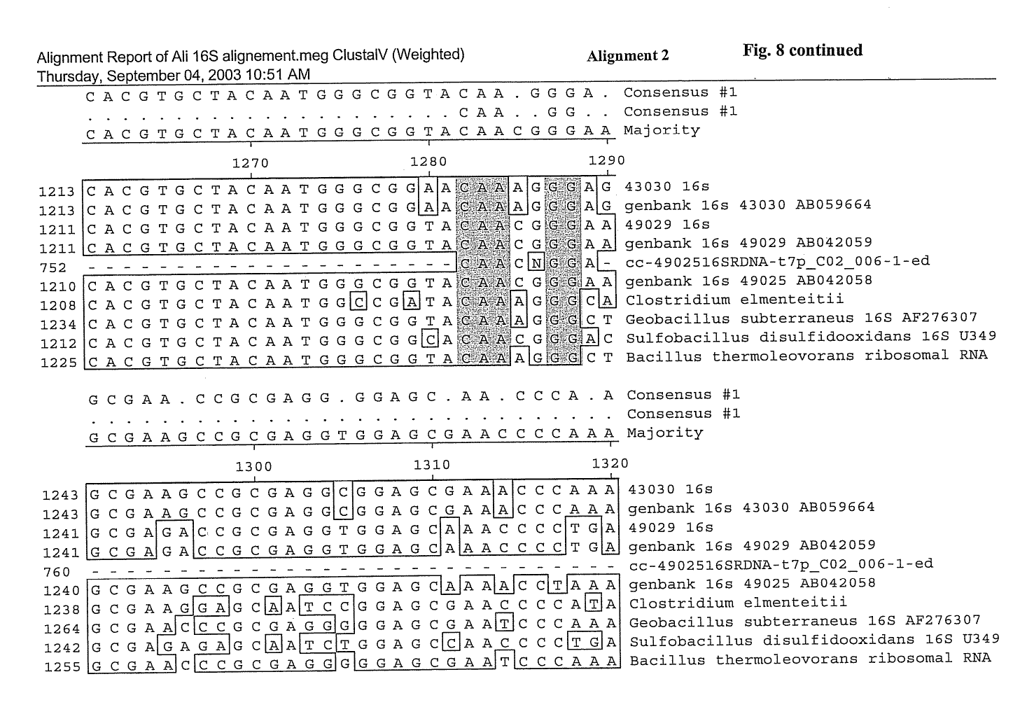

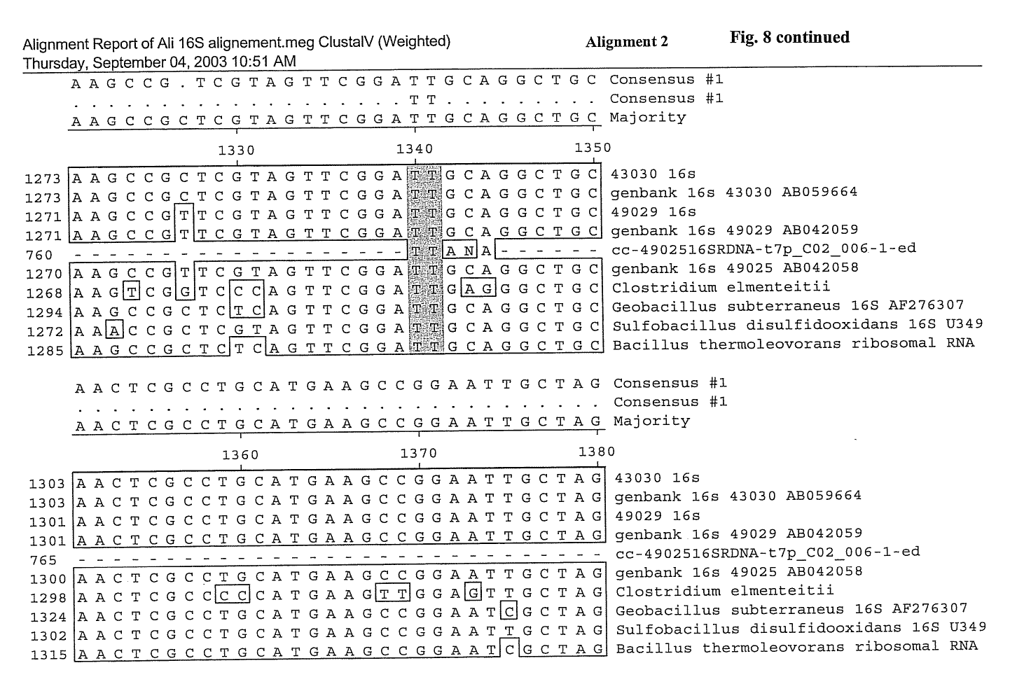

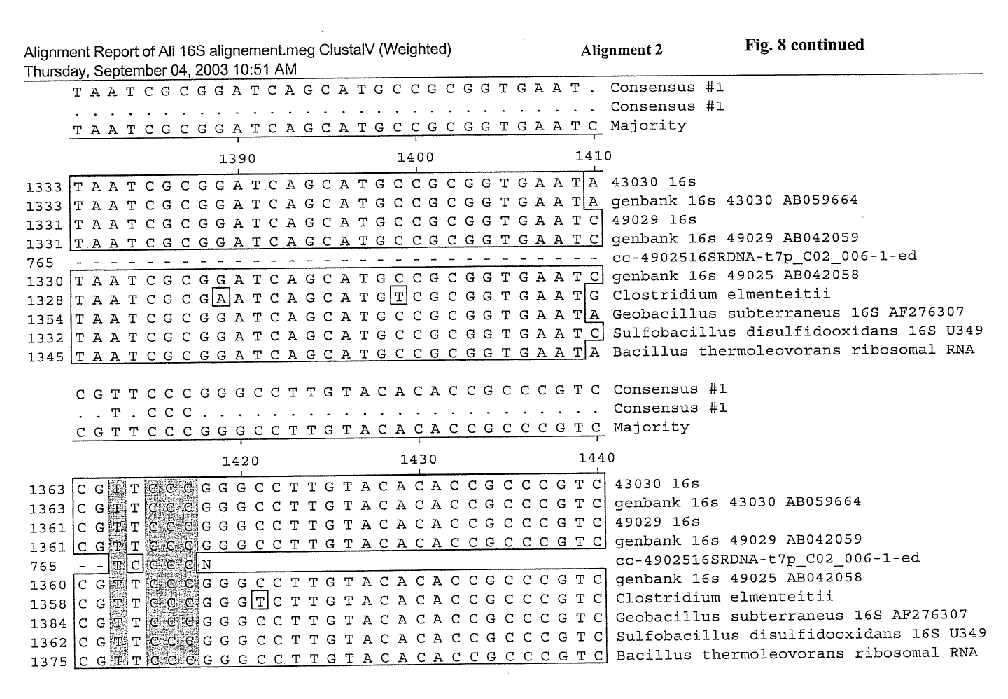

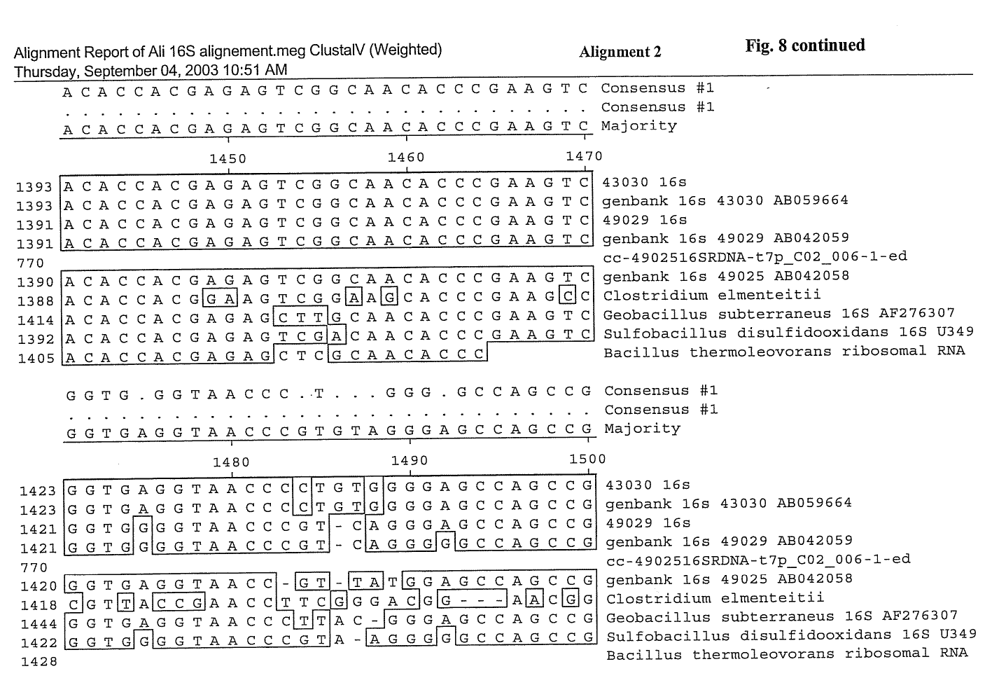

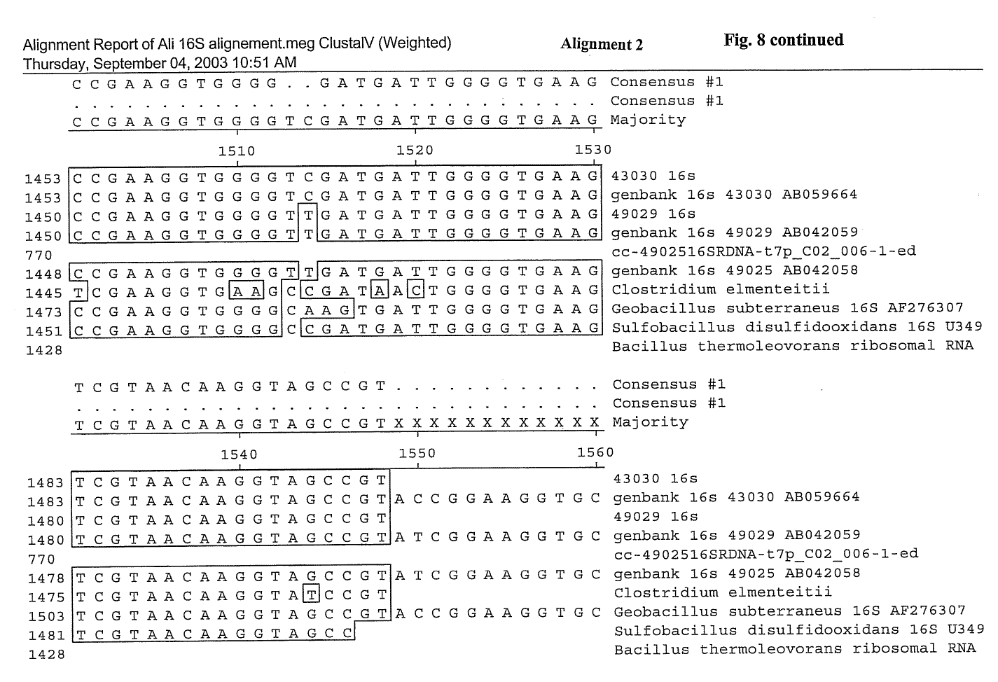

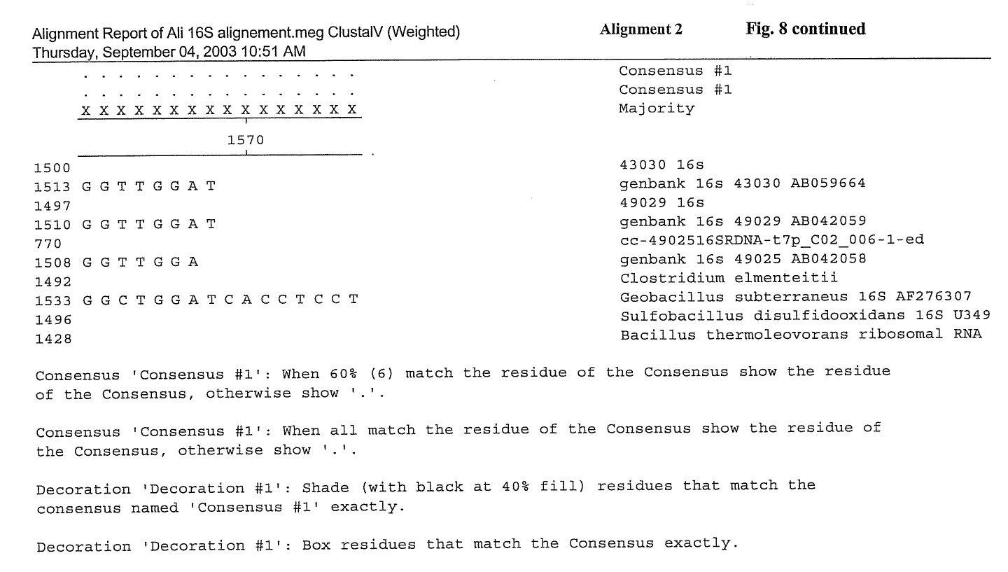

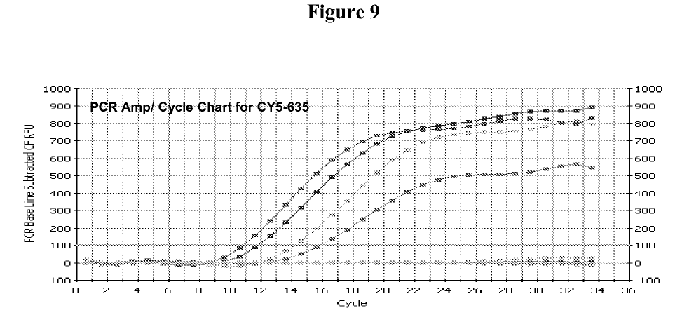

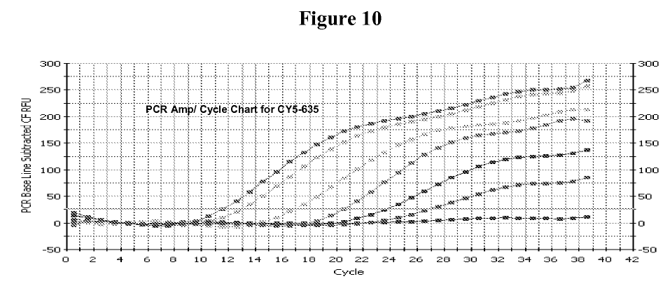

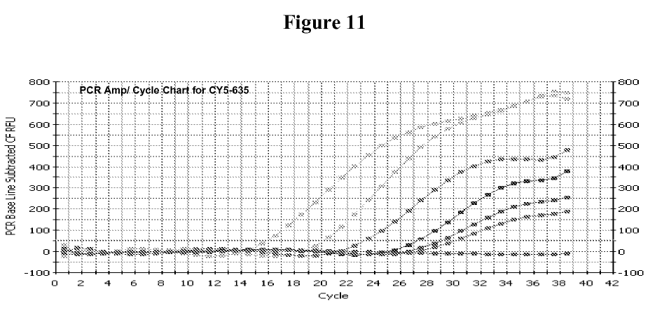

Using an established approach (Wang et al., 2001), squalene-hopene cyclase protein sequences from several microorganisms were aligned and conserved amino acid sequences in squalene-hopene cyclase were identified. FIGS. 5 and 6, respectively, show the polynucleotide and protein alignments for two strains of Alicyclobacillus. Two degenerate primers 5′ GGNGGNTGGATGTTYCARGC 3′ (Y<C+T; R=A+G; N=A+T+C+G) (SEQ ID NO 64) and 5′ YTCNCCCCANCCNCCRTC 3′ (SEQ ID NO 65) were derived. Using this set of primers and the genomic DNA from A. acidocaldarius ATCC 43030 and A. acidoterrestris ATCC 49025, the 705 bp shc fragments were amplified by PCR from both strains. The PCR fragments were cloned into the TA vector and the inserted DNA sequences were determined. The DNA sequences were further compared with other Alicyclobacillus spp. shc sequences in the GenBank. Three conserved oligonucleotides were derived including the Forward Primer 5′ ATGCAGAGYTCGAACG 3′ (SEQ ID NO 25) and the Reverse Primer 5′ AAGCTGCCGAARCACTC 3′ (SEQ ID NO 27) flanking a 136 bp fragment, and the Probe 5′TCRGARGACGTCACCGC3′ (SEQ ID NO 26). The synthesized primers were ordered from Sigma-Genosys (The Woodlands, Tex.). The Probe is fluorescence-labeled with 5′ 6-FAM BHQ-1 3′ by Biosearch Technologies, Inc. (Novato, Calif.) and was used in the Taqman assay.

### Specific Detection of Spoilage Alicyclobacillus spp.

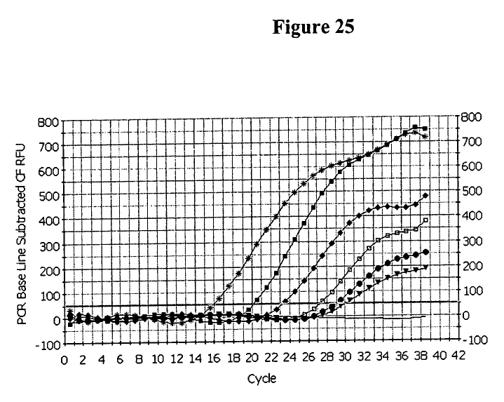

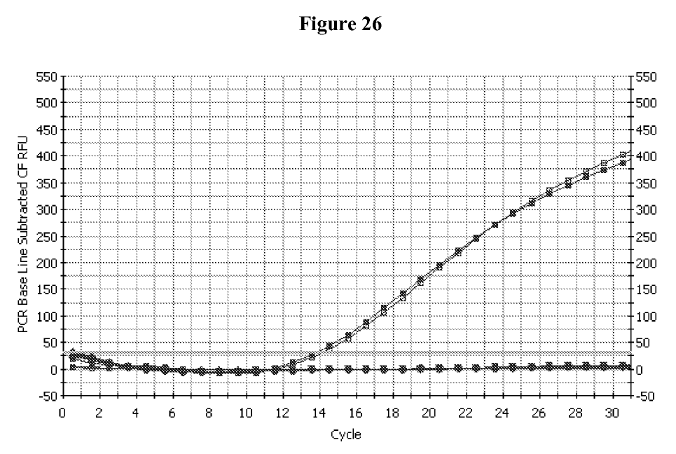

Real-time PCR assays were performed to determine the specificity of the primers and probe for spoilage Alicyclobacillus spp. E. coli DH-5α, L. lactis subsp. lactis C2, and P. putida 49L/51, G. stearothermophilus ATCC 10149 were also included in the study to test the possibility of cross-reactions by the primer-and-probe set with common food-borne microorganisms. Assays were performed in triplicate, and a representative real-time PCR curve plotted by the iCycler iQ Optical System Software is shown in FIG. 26.
Representative strains from A. acidocaldarius and A. acidoterrestris were tested positive. No cross-reaction was detected in other commonly found food-borne microorganisms. Further specificity study was conducted by searching the Blast databases for DNA sequences from the National Center for Biotechnology Information (NCBI). We found no combination of the above three oligonucleotides in other microorganisms but A. acidocaldarius and A. acidoterrestris. The data suggested that the system is specific for spoilage A. acidocaldarius and A. acidoterrestris.

### Levels of Detection in Bacterial Culture Medium and in Apple Juice.

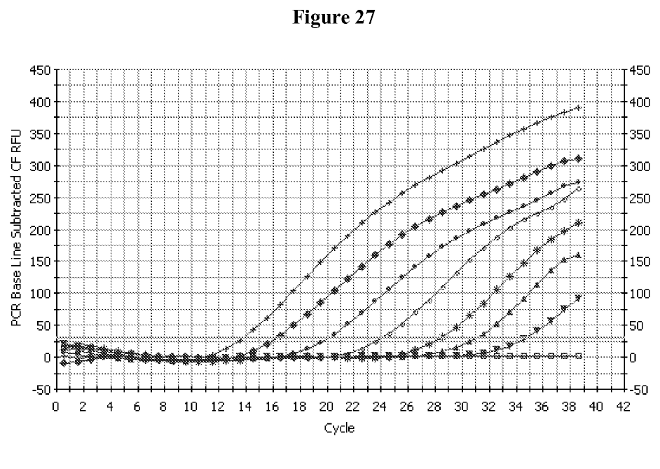

To establish the detection level using the above real-time PCR system, we have conducted 10⁰ to 10⁻⁶ serial dilutions of A. acidoterrestris ATCC 49025 in culture medium. Cells from 1 ml of diluted samples were collected and 10/100 of the DNAs extracted were used as template in the real-time PCR analysis. All experiments were repeated for at least three times and a representative curve was presented as FIG. 27. Our results showed that using the above primer-and-probe set, the presence of as few as 10 cells in a sample could be detected. This detection level is comparable to results from other microbial detection studies using real-time PCR.

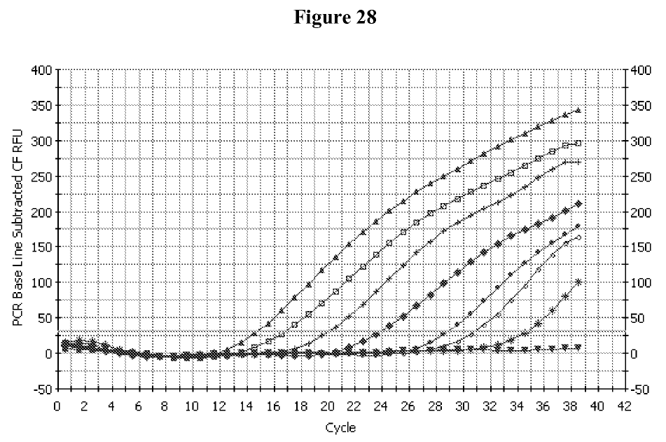

To further verify the feasibility of using the detection system in juice products, we have conducted 10⁰ to 10⁻⁶ serial dilutions of A. acidoterrestris ATCC 49025 in apple juice. The experiments were repeated for three times and a representative curve was presented as FIG. 28. Similar detection level was achieved in apple juice.

### 2. DISCUSSION AND CONCLUSION

Rapid, specific and sensitive detection of microorganisms in agricultural and food systems has proved to be a challenge. There are several major hurdles for effective microbial detection in the food systems. First, problematic food is normally associated with low level of initial contamination. However, the rich food matrix can support the growth of microbial agents in many cases during food storage and distribution. Thus even low level of initial contamination can cause serious damage. To be able to detect the presence of this low level contamination from food matrix often involving bulk proteins, carbohydrates and fatty acids, proper sampling and lengthy pre-detection enrichment steps are often required. To achieve rapid detection, pre-detection enrichment procedures need to be minimized and the detection system also should be sensitive enough to pick up low level of contamination.

Second, both foods and farm environment are complex ecosystems with significant background microflora. In addition to the background microflora normally associated with raw materials, beneficial microorganisms such as starter cultures sometimes are intentionally inoculated and present in large quantity in certain products. Therefore, to avoid false positive results, detection method for spoilage or pathogenic organisms needs to be specific enough to pick up only the target microorganisms. Finally, the rich and complex food ingredients often include various salts, carbohydrates, preservatives, emulsifiers, fatty acids, and proteins. The presence of these components varies among food commodities and can interfere with detection in various degrees. Therefore detection approaches and procedures need to be verified for effectiveness in these food systems.

Real-time Taqman PCR-based approach has the potential to achieve rapid, sensitive and specific detection. An average DNA amplification cycle for a small fragment can be completed within a minute. Theoretically after 30-40 cycles the amplification products from one DNA template in the system can be readily detected and plotted on the screen in almost real-time. The double sequence selection mechanism involving both the oligonucleotide primers and probe further minimizes the possibility of false positive results and enhances the detection specificity.

In this study, using a primer-and-probe set targeting the spoilage A. acidocaldarius and A. acidoterrestris, we were able to achieve specific detection without cross-reacting with representative strains from other common food-borne microorganisms including a strain from the closely related thermophilic G. stearothermophilus. Although only a few representative strains were used in the laboratory specificity studies, a computer-based search covering all the world-wide deposited DNA sequences available through the NCBI website was conducted to ensure that the combination of the sequences of the oligonucleotide primers and probe used in the study are distinctive enough to detect only A. acidocaldarius and A. acidoterrestris strains.

The level of detection limit with confidence is important for any detection approaches. In this study we have conducted sensitivity tests in both bacterial cultural medium and a real food system-apple juice. For laboratory handling purpose and for the convenient of using commercially available yet economically feasible DNA extraction kit, bacterial cells were serially diluted in either medium or juice and cells in 1 ml of samples were collected by micro-centrifugation. DNA were extracted and 10/100 of the elution were used as template in PCR. The experiment was repeated at least three times and a representative curve presented as FIG. 27. The lowest detection limit was determined based on the cell count numbers from agar plates derived from dilution with the optimal counting numbers (30-300) and the fold of dilution corresponding to each positive curves presented. Using this approach, we report that the presence of as few as 10 cells per sample with confidence. Because during each independent repeats the 10-fold serial dilutions were conducted without knowing exactly how many cells were in 1 ml of samples, the standard deviation reflects this fact. To further narrow down the range of standard deviation of detection, serial dilutions within the range of 2-10 can be conducted so a more precise confident level can be possibly established. We did not extrapolate the results using In other referred paper sometimes a standard curve was established first for sensitivity analysis. Furthermore, in a quality control laboratory, a regular sample size is normally 25 ml instead of 1 ml. Theoretically, sample detection limits can further be improved as long as cells from 25 ml or even 100 ml of samples can be properly collected and re-suspended in 1 ml of solution to conduct DNA extraction.

We are in the process of establishing a rapid detection system for food industry applications (the CleanPlant system) and the real-time Taqman PCR is one of our preferred platforms. In order to apply this detection platform in juice related products, we need to establish the feasibility of using the system for raw material screening and final product monitoring. We have conducted the sensitivity test by spiking the Alicyclobacillus in apple juice purchased from local grocery stores and similar level of detection was achieved indicating the applicability of such a system in final product screening. Further, we have used this system to detect the presence of Alicyclobacillus in apple juice concentrates, which are considered raw materials for the processing facilities. Similar level of detection was achieved except diluting and rinsing procedures need to be incorporated to minimize inhibitory effects by the concentrated food ingredients (data not shown). These data suggested that

Because the system we developed is based on recognition of the signature DNA sequence of microorganisms, it has high specificity and does not cross react with other food-borne microorganisms (FIG. 26). The detection limit was achieved in both bacterial culture medium and apple juice. Since no inhibition to the reaction system was detected using samples collected from apple juice, we expect the sensitivity of the detection system can be further improved by including a pre-treatment procedure to apply a centrifugation or membrane filtration procedure to concentrate the bacteria cells from a large sample volume. This approach is in fact a preferred practice in the industry where the sampling size varies from 25 ml to 1 liter. Since only 1/10 of the DNA extract was used in the reaction, we expect further improvement for the sensitivity can be achieved by incorporating more DNA template to the reaction system.

### Example 3

### Yeast Genomic DNA Extraction Protocol

Innoculate yeast, overnight; Centrifuge 10,000 rpm for 10 mins; Discard supernatant, add 600 ul Sorbital buffer (1 M Sorbital, 100 mM EDTA, 14 mM B-mercaptoethanol, 30 ul 20 mg/ml lyticase) in pellet, vortex, room temperature for 30 min; Centrifuge 10,000 rpm for 5 min; Add 180 ATL (Qiagen DNAeasy kit) and 20 ul proteinase K (Qiagen DNAeasy kit) to pellet and vortex; 55°. for 1 h, add 200 ul AL (Qiagen DNAeasy kit), 70°. for 10 min; 200 ul Ethanol, vortex, apply to DNeasy spin column.; centrifuge 10,000 rpm for 1 min, discard flow-through add 500 ul Buffer AW1 (Qiagen DNAeasy kit), spin for 1 min; add 500 ul Buffer AW1 (Qiagen DNAeasy kit), spin for 3 min; add 100 ul AE buffer (Qiagen DNAeasy kit), spin for 1 min.

### Mold Genomic DNA Extraction Protocol:

Innoculate Mold in PDB; 3 days later, centrifuge 10,000 rpm for 10 min; add 500 ul Mold extraction buffer (1% CTAB, 1.4 M NaCl, 100 mM Tris, 20 mM EDTA, pH 8.0) to pellet; 100 ul glass beads, water bath sonic (550.) for 45 min; add 50 ul Proteinase K (Qiagen DNAeasy kit) and incubate in 55°. for 1 h; Centrifuge 10,000 rpm for 5 min; Transfer the supernatant, add 500 ul AL (Qiagen DNAeasy kit), 70°. for 10 min; Add 200 ul Ethanol and pipet it into Dneasy mini column; 10,000 rpm for 1 min; Add 500 ul AW1 (Qiagen DNAeasy kit), spin for 1 min; Add 500 ul AW2 (Qiagen DNAeasy kit), spin for 3 min; Add 100 AE buffer (Qiagen DNAeasy kit), spin for 1 min.

1. A method for detecting Alicyclobacillus and Geobacillus in a test sample, the method comprising
(a) providing an oligonucleotide set comprising:
(i) a forward primer of from 15 to 35 nucleotides in length, said forward primer comprising a sequence which is capable of hybridizing to a first consecutive sequence within the sequence of nucleotide position 1327 through nucleotide position 1460 of SEQ ID NO: 78;
(ii) a reverse primer of from 15 to 35 nucleotides in length, said reverse primer comprising a sequence which is capable of hybridizing to the inverse complement of a second consecutive sequence within the sequence of nucleotide position 1327 through nucleotide position 1460 of SEQ ID NO: 78, said second consecutive sequence being downstream from said first consecutive sequence; and
(iii) a probe of from at least 15 nucleotides in length, said probe comprising a sequence which is which is capable of hybridizing to a third consecutive sequence within the sequence of nucleotide position 1327 through nucleotide position 1460 of SEQ ID NO: 78, or the complement thereof, and falls between or overlaps with the first and second consecutive sequences;
(b) amplifying DNA in the sample with the said primer set and a polymerase chain reaction, and
(c) determining the presence of PCR products of step (b), wherein the presence of a PCR product in the sample is indicative of contamination of the test sample by Alicyclobacillus or Geobacillus or both.

2. The method of claim 1 wherein the forward primer has a sequence selected from the group consisting of SEQ ID NO 1, SEQ ID NO 5, SEQ ID NO 9, SEQ ID NO 13, SEQ ID NO 17, and SEQ ID NO 20.

3. The method of claim 1 wherein the reverse primer has a sequence selected from the group consisting of SEQ ID NO 4, SEQ ID NO 8, SEQ ID NO 12, SEQ ID NO 16, SEQ ID NO 19, and SEQ ID NO 23.

4. The method of claim 1 wherein the probe has a sequence selected from the group consisting of SEQ ID NO 2, SEQ ID NO 3, SEQ ID NO 6, SEQ ID NO 7, SEQ ID NO 10, SEQ ID NO 1, SEQ ID NO 14, SEQ ID NO 15, SEQ ID NO 18, SEQ ID NO 21, and SEQ ID NO 22.

5. A method for detecting Alicyclobacillus and Geobacillus in a test sample, the method comprising
(a) providing a oligonucleotide set comprising:
(i) a forward primer of from 15 to 35 nucleotides in length, said forward primer comprising a sequence which is capable of hybridizing to a first consecutive sequence within the sequence of nucleotide position 334 through nucleotide position 485 of SEQ ID NO: 140;
(ii) a reverse primer of from 15 to 35 nucleotides in length, said reverse primer comprising a sequence which is capable of hybridizing to the inverse complement of a second consecutive sequence within the sequence of nucleotide position 334 through nucleotide position 485 of SEQ ID NO: 140, said second consecutive sequence being downstream from said first consecutive sequence; and
(iii) a probe of from at least 15 nucleotides in length, said probe comprising a sequence which is which is capable of hybridizing to a third consecutive sequence within the sequence of nucleotide position 334 through nucleotide position 485 of SEQ ID NO: 140, or the complement thereof, and falls between or overlaps with the first and second consecutive sequences;
(b) amplifying DNA in the sample with the said primer set and a polymerase chain reaction, and
(c) determining the presence of PCR products of step (b), wherein the presence of a PCR product in the sample is indicative of contamination of the test sample by Alicyclobacillus or Geobacillus or both.

6. The method of claim 5 wherein the forward primer has a sequence selected from the group consisting of SEQ ID NO 28, and SEQ ID NO 29.

7. The method of claim 5 wherein the reverse primer has a sequence selected from the group consisting of SEQ ID NO 34, SEQ ID NO 35, SEQ ID NO 36, and SEQ ID NO 37.

8. The method of claim 5 wherein the probe has a sequence selected from the group consisting of SEQ ID NO 30, SEQ ID NO 31, SEQ ID NO 32, and SEQ ID NO 33.

9. A method for detecting Alicyclobacillus and Geobacillus in a test sample, the method comprising
(a) providing a oligonucleotide set comprising:
(i) a forward primer of from 15 to 35 nucleotides in length, said forward primer comprising a sequence which is capable of hybridizing to a first consecutive sequence within the sequence of nucleotide position 752 through nucleotide position 813 of SEQ ID NO: 78;
(ii) a reverse primer of from 15 to 35 nucleotides in length, said reverse primer comprising a sequence which is capable of hybridizing to the inverse complement of a second consecutive sequence within the sequence of nucleotide position 752 through nucleotide position 813 of SEQ ID NO: 78, said second consecutive sequence being downstream from said first consecutive sequence; and
(iii) a probe of from at least 15 nucleotides in length, said probe comprising a sequence which is which is capable of hybridizing to a third consecutive sequence within the sequence of nucleotide position 752 through nucleotide position 813 of SEQ ID NO: 78, or the complement thereof, and falls between or overlaps with the first and second consecutive sequences;
(b) amplifying DNA in the sample with the said primer set and a polymerase chain reaction, and
(c) determining the presence of PCR products of step (b), wherein the presence of a PCR product in the sample is indicative of contamination of the test sample by Alicyclobacillus or Geobacillus or both.

10. The method of claim 9 wherein the forward primer has a sequence selected from the group consisting of SEQ ID NO 38, SEQ ID NO 39, SEQ ID NO 40, SEQ ID NO 41, SEQ ID NO 42, and SEQ ID NO 43.

11. The method of claim 9 wherein the reverse primer has a sequence selected from the group consisting of SEQ ID NO 50, SEQ ID NO 51, SEQ ID NO 52, and SEQ ID NO 53.

12. The method of claim 9 wherein the probe has a sequence selected from the group consisting of SEQ ID NO 44, SEQ ID NO 45, SEQ ID NO 46, SEQ ID NO 47, SEQ ID NO 48, and SEQ ID NO 49.

13. A method for detecting mold or yeast in a test sample, the method comprising
(a) providing a oligonucleotide set comprising:
(i) a forward primer of from 15 to 35 nucleotides in length, said forward primer comprising a sequence which is capable of hybridizing to a first consecutive sequence within the sequence of nucleotide position 81 through nucleotide position 225 of SEQ ID NO: 123;
(ii) a reverse primer of from 15 to 35 nucleotides in length, said reverse primer comprising a sequence which is capable of hybridizing to the inverse complement of a second consecutive sequence within the sequence of nucleotide position 81 through nucleotide position 225 of SEQ ID NO: 123, said second consecutive sequence being downstream from said first consecutive sequence; and
(iii) a probe of from at least 15 nucleotides in length, said probe comprising a sequence which is which is capable of hybridizing to a third consecutive sequence within the sequence of nucleotide position 81 through nucleotide position 225 of SEQ ID NO: 123, or the complement thereof, and falls between or overlaps with the first and second consecutive sequences;
(b) amplifying DNA in the sample with the said primer set and a polymerase chain reaction, and
(c) determining the presence of PCR products of step (b), wherein the presence of a PCR product in the sample is indicative of contamination of the test sample by mold or yeast or both.

14. The method of claim 13 wherein the forward primer has a sequence selected from the group consisting of SEQ ID NO 54, and SEQ ID NO 58.

15. The method of claim 13 wherein the reverse primer has a sequence selected from the group consisting of SEQ ID NO 55, and SEQ ID NO 59.

16. The method of claim 13 wherein the probe has a sequence selected from the group consisting of SEQ ID NO 56, SEQ ID NO 57, and SEQ ID NO 60.

17. A method for detecting mold or yeast in a test sample, the method comprising
(a) providing a oligonucleotide set comprising:
(i) a forward primer of from 15 to 35 nucleotides in length, said forward primer comprising a sequence which is capable of hybridizing to a first consecutive sequence within the sequence of nucleotide position 114 through nucleotide position 238 of SEQ ID NO: 123;
(ii) a reverse primer of from 15 to 35 nucleotides in length, said reverse primer comprising a sequence which is capable of hybridizing to the inverse complement of a second consecutive sequence within the sequence of nucleotide position 114 through nucleotide position 238 of SEQ ID NO: 123, said second consecutive sequence being downstream from said first consecutive sequence; and
(iii) a probe of from at least 15 nucleotides in length, said probe comprising a sequence which is which is capable of hybridizing to a third consecutive sequence within the sequence of nucleotide position 114 through nucleotide position 238 of SEQ ID NO: 123, or the complement thereof, and falls between or overlaps with the first and second consecutive sequences;
(b) amplifying DNA in the sample with the said primer set and a polymerase chain reaction, and
(c) determining the presence of PCR products of step (b), wherein the presence of a PCR product in the sample is indicative of contamination of the test sample by mold or yeast or both.

18. The method of claim 17 wherein the forward primer has a sequence of SEQ ID NO 61.

19. The method of claim 17 wherein the reverse primer has a sequence of SEQ ID NO 62.

20. The method of claim 17 wherein the probe has a sequence of SEQ ID NO 63.

21. The method of claim 1 wherein the primers
i) do not contain runs of more than 5 of the same nucleotide base,
ii) do not contain internal palindromic sequences,
iii) do not hybridize to one another under stringent conditions, and
iv) have 40 to 60 percent G+C content, and
wherein said PCR amplification provides a PCR product that is from 50 to 613 nucleotides in length.

22. The method of claim 1, wherein the PCR is quantitative PCR.

23. The method of claim 1, wherein the PCR is real-time PCR.

24. A method of detecting the presence of acidic bacteria in a test sample using real time monitoring of a polymerase chain reaction amplification of a target nucleic acid sequence found in the acidic bacteria, said method comprising the steps of
(a) adding to the test sample an effective amount of a forward nucleic acid primer and reverse nucleic acid primer and a nucleic acid probe, wherein the forward primer is selected from the group consisting of SEQ ID NO 1, SEQ ID NO 5, SEQ ID NO 9, SEQ ID NO 13, SEQ ID NO 17, and SEQ ID NO 20, and wherein the reverse primer is selected from the group consisting of SEQ ID NO 4, SEQ ID NO 8, SEQ ID NO 12, SEQ ID NO 16, SEQ ID NO 19, and SEQ ID NO 23, and wherein the probe is selected from the group consisting of SEQ ID NO 2, SEQ ID NO 3, SEQ ID NO 6, SEQ ID NO 7, SEQ ID NO 10, SEQ ID NO 11, SEQ ID NO 14, SEQ ID NO 15, SEQ ID NO 18, SEQ ID NO 21, and SEQ ID NO 22 and wherein the probe hybridizes to an amplified copy of the target nucleic acid sequence, and wherein the probe is labeled with a marker which emits a signal upon the hybridization of the probe to the target nucleic acid sequence;
(b) amplifying the target nucleic acid sequence by polymerase chain reaction;
(c) detecting the emitted signal of the sample.

25. A method of detecting the presence of fungi in a test sample using real time monitoring of a polymerase chain reaction amplification of a target nucleic acid sequence found in the acidic bacteria, said method comprising the steps of
(a) adding to the test sample an effective amount of a forward nucleic acid primer and reverse nucleic acid primer and a nucleic acid probe, wherein the forward primer is selected from the group consisting of SEQ ID NO 54, and SEQ ID NO 58 and SEQ ID NO 61, and wherein the reverse primer is selected from the group consisting of SEQ ID NO 55, SEQ ID NO 59, and SEQ ID NO 62, and wherein the probe is selected from the group consisting of SEQ ID NO 56, SEQ ID NO 57, SEQ ID NO 60, and SEQ ID NO 63 and wherein the probe hybridizes to an amplified copy of the target nucleic acid sequence, and wherein the probe is labeled with a marker which emits a signal upon the hybridization of the probe to the target nucleic acid sequence;
(b) amplifying the target nucleic acid sequence by polymerase chain reaction;
(c) detecting the emitted signal of the sample.

26. A kit for detecting Alicyclobacillus and Geobacillus in a test sample, comprising a set of oligonucleotides comprising:
(a) a forward primer of from 15 to 35 nucleotides in length, said forward primer comprising a sequence which is capable of hybridizing to a first consecutive sequence within the sequence of nucleotide position 1327 through nucleotide position 1460 of SEQ ID NO: 78;
(b) a reverse primer of from 15 to 35 nucleotides in length, said reverse primer comprising a sequence which is capable of hybridizing to the inverse complement of a second consecutive sequence within the sequence of nucleotide position 1327 through nucleotide position 1460 of SEQ ID NO: 78, said second consecutive sequence being downstream from said first consecutive sequence; and
(c) a probe of from at least 15 nucleotides in length, said probe comprising a sequence which is which is capable of hybridizing to a third consecutive sequence within the sequence of nucleotide position 1327 through nucleotide position 1460 of SEQ ID NO: 78, or the complement thereof, and falls between or overlaps with the first and second consecutive sequences.

27. A kit for detecting Alicyclobacillus and Geobacillus in a test sample, comprising a set of oligonucleotides comprising:
(a) a forward primer of from 15 to 35 nucleotides in length, said forward primer comprising a sequence which is capable of hybridizing to a first consecutive sequence within the sequence of nucleotide position 334 through nucleotide position 485 of SEQ ID NO: 140;
(b) a reverse primer of from 15 to 35 nucleotides in length, said reverse primer comprising a sequence which is capable of hybridizing to the inverse complement of a second consecutive sequence within the sequence of nucleotide position 334 through nucleotide position 485 of SEQ ID NO: 140, said second consecutive sequence being downstream from said first consecutive sequence; and
(c) a probe of from at least 15 nucleotides in length, said probe comprising a sequence which is which is capable of hybridizing to a third consecutive sequence within the sequence of nucleotide position 334 through nucleotide position 485 of SEQ ID NO: 140, or the complement thereof, and falls between or overlaps with the first and second consecutive sequences.

28. A kit for detecting Alicyclobacillus and Geobacillus in a test sample, comprising a set of oligonucleotides comprising:
(a) a forward primer of from 15 to 35 nucleotides in length, said forward primer comprising a sequence which is capable of hybridizing to a first consecutive sequence within the sequence of nucleotide position 752 through nucleotide position 813 of SEQ ID NO: 140;
(b) a reverse primer of from 15 to 35 nucleotides in length, said reverse primer comprising a sequence which is capable of hybridizing to the inverse complement of a second consecutive sequence within the sequence of nucleotide position 752 through nucleotide position 813 of SEQ ID NO: 140, said second consecutive sequence being downstream from said first consecutive sequence; and
(c) a probe of from at least 15 nucleotides in length, said probe comprising a sequence which is which is capable of hybridizing to a third consecutive sequence within the sequence of nucleotide position 752 through nucleotide position 813 of SEQ ID NO: 140, or the complement thereof, and falls between or overlaps with the first and second consecutive sequences.

29. A kit for detecting yeast or mold in a test sample, comprising a set of oligonucleotides comprising:
(a) a forward primer of from 15 to 35 nucleotides in length, said forward primer comprising a sequence which is capable of hybridizing to a first consecutive sequence within the sequence of nucleotide position 81 through nucleotide position 225 of SEQ ID NO: 123;
(b) a reverse primer of from 15 to 35 nucleotides in length, said reverse primer comprising a sequence which is capable of hybridizing to the inverse complement of a second consecutive sequence within the sequence of nucleotide position 81 through nucleotide position 225 of SEQ ID NO: 123, said second consecutive sequence being downstream from said first consecutive sequence; and
(c) a probe of from at least 15 nucleotides in length, said probe comprising a sequence which is which is capable of hybridizing to a third consecutive sequence within the sequence of nucleotide position 81 through nucleotide position 225 of SEQ ID NO: 123, or the complement thereof, and falls between or overlaps with the first and second consecutive sequences.

30. A kit for detecting yeast or mold in a test sample, comprising a set of oligonucleotides comprising:
(a) a forward primer of from 15 to 35 nucleotides in length, said forward primer comprising a sequence which is capable of hybridizing to a first consecutive sequence within the sequence of nucleotide position 114 through nucleotide position 238 of SEQ ID NO: 123;
(b) a reverse primer of from 15 to 35 nucleotides in length, said reverse primer comprising a sequence which is capable of hybridizing to the inverse complement of a second consecutive sequence within the sequence of nucleotide position 114 through nucleotide position 238 of SEQ ID NO: 123, said second consecutive sequence being downstream from said first consecutive sequence; and
(c) a probe of from at least 15 nucleotides in length, said probe comprising a sequence which is which is capable of hybridizing to a third consecutive sequence within the sequence of nucleotide position 114 through nucleotide position 238 of SEQ ID NO: 123, or the complement thereof, and falls between or overlaps with the first and second consecutive sequences.
